(19) United States
(12) Patent Application Publication
(10) Pub. No.: US 2010/0261736 A1
(43) Pub. Date: Oct. 14, 2010

(54) SUBSTITUTED BICYCLIC HETEROARYL COMPOUNDS FOR THE TREATMENT OF CARDIOVASCULAR DISEASE

(76) Inventors: Thomas Lampe, Dusseldorf (DE); Raimund Kast, Wuppertal (DE); Hartmut Beck, Koln (DE); Friederike Stoll, Dusseldorf (DE); Eva-Maria Becker, Wuppertal (DE); Mario Jeske, Solingen (DE); Joachim Schuhmacher, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(21) Appl. No.: 12/664,788

(22) PCT Filed: Jun. 3, 2008
(86) PCT No.: PCT/EP08/04408

§ 371 Date: Jun. 14, 2010

(30) Foreign Application Priority Data
Jun. 16, 2007 (DE) 10 2007 027 800.6

(51) Int. Cl. A61K 31/519; C07D 495/04; A61P 9/12; A61P 7/02; C07D 487/04; C07D 307/80; A61K 31/343; C07D 491/048; A61K 31/4355

(52) U.S. Cl. 514/260.1; 544/278; 514/265.1; 544/280; 549/469; 514/469; 546/116; 514/302

(57) ABSTRACT

The present application relates to novel substituted bicyclic heteroaryl compounds, to processes for their preparation, to their use for the treatment and/or prophylaxis of diseases and to their use for preparing medicaments for the treatment and/or prophylaxis of diseases, in particular for the treatment and/or prophylaxis of cardiovascular diseases.

The present application relates to novel substituted bicyclic heteroaryl compounds, to processes for their preparation, to their use for the treatment and/or prophylaxis of diseases and to their use for preparing medicaments for the treatment and/or prophylaxis of diseases, in particular for the treatment and/or prophylaxis of cardiovascular diseases.

Prostacyclin (PGI₂) belongs to the class of bioactive prostaglandins, which are derivatives of arachidonic acid. PGI₂ is the main product of arachidonic acid metabolism in endothelial cells and is a potent vasodilator and inhibitor of platelet aggregation. PGI₂ is the physiological antagonist of thromboxane A₂ (TxA₂), a strong vasoconstrictor and stimulator of thrombocyte aggregation, and thus contributes to the maintenance of vascular homeostasis. A drop in PGI₂ levels is presumed to be partly responsible for the development of various cardiovascular diseases [Dusting, G. J. et al., Pharmac. Ther. 1990, 48: 323-344; Vane, J. et al., Eur. J. Vasc. Endovasc. Surg. 2003, 26: 571-578].

After release of arachidonic acid from phospholipids via phospholipases A₂, PGI₂ is synthesized by cyclooxygenases and then by PGI₂-synthase. PGI₂ is not stored, but is released immediately after synthesis, exerting its effects locally. PGI₂ is an unstable molecule, which is transformed rapidly (half-life approx. 3 minutes) and non-enzymatically, to an inactive metabolite, 6-keto-prostaglandin-F1alpha [Dusting, G. J. et al., Pharmac. Ther. 1990, 48: 323-344].

The biological effects of PGI₂ occur through binding to a membrane-bound receptor, called the prostacyclin receptor or IP receptor [Narumiya, S. et al., Physiol. Rev. 1999, 79: 1193-1226]. The IP receptor is one of the G-protein-coupled receptors, which are characterized by seven transmembrane domains. In addition to the human IP receptor, prostacyclin receptors have also been cloned from rat and mouse [Vane, J. et al., Eur. J. Vasc. Endovasc. Surg. 2003, 26: 571-578]. In smooth muscle cells, activation of the IP receptor leads to stimulation of adenylate cyclase, which catalyses the formation of cAMP from ATP. The increase in the intracellular cAMP concentration is responsible for prostacyclin-induced vasodilation and for inhibition of platelet aggregation. In addition to the vasoactive properties, anti-proliferative effects [Schroer, K. et al., Agents Actions Suppl. 1997, 48: 63-91; Kothapalli, D. et al., Mol. Pharmacol. 2003, 64: 249-258; Planchon, P. et al., Life Sci. 1995, 57: 1233-1240] and anti-arteriosclerotic effects [Rudic, R. D. et al., Circ. Res. 2005, 96: 1240-1247; Egan K. M. et al., Science 2004, 114: 784-794] have also been described for PGI₂. Furthermore, PGI₂ also inhibits the formation of metastases [Schneider, M. R. et al., Cancer Metastasis Rev. 1994, 13: 349-64]. It is unclear whether these effects are due to stimulation of cAMP formation or to IP receptor-mediated activation of other signal transduction pathways in the respective target cell [Wise, H. et al. TIPS 1996, 17: 17-21], such as the phosphoinositide cascade, and of potassium channels.

Although the effects of PGI₂ are on the whole of benefit therapeutically, clinical application of PGI₂ is severely restricted by its chemical and metabolic instability. PGI₂ analogs that are more stable, for example iloprost [Badesch, D. B. et al., J. Am. Coll. Cardiol. 2004, 43: 56S-61S] and treprostinil [Chattaraj, S. C., Curr. Opion. Invest. Drugs 2002, 3: 582-586] have been made available, but these compounds still have a very short time of action. Moreover, the substances can only be administered to the patient via complicated routes of administration, e.g. by continuous infusion, subcutaneously or via repeated inhalations. These routes of administration can also have additional side-effects, for example infections or pains at the site of injection. The use of beraprost, which to date is the only PGI₂ derivative available for oral administration to the patient [Barst, R. J. et al., J. Am. Coll. Cardiol. 2003, 41: 2119-2125], is once again limited by its short time of action.

The compounds described in the present application are, compared with PGI₂, chemically and metabolically stable, non-prostanoid activators of the IP receptor, which imitate the biological action of PGI₂ and can thus be used for treating diseases, in particular cardiovascular diseases.

WO 00/75145 claims compounds having a bicyclic heteroaryl core structure as inhibitors of cell adhesion. 4-Amino-, 4-oxy- and/or 4-thio-substituted furo [2,3-d]-pyrimidine, thieno[2,3-d]pyrimidine and/or pyrrolo[2,3-d]pyrimidine derivatives and their use for treating diseases are disclosed inter alia in WO 97/02266, WO 99/07703, WO 99/65908, JP 2002-105081-A, WO 02/092603, WO 03/018589, WO 03/022852, WO 2004/111057, WO 2005/092896, WO 2005/121149, WO 2006/004658, WO 2006/004703 and US 2007/0099877-A1. The preparation and biological activity of diaryl-substituted thieno[2,3-d]pyrimidines having 4-glycine or 4-alanine side chains is reported in Bioorg. Med. Chem. 10, 3113-3122 (2002). Various 4-amino-, 4-oxy- and/or 4-thio-substituted furopyridine, thienopyridine or pyrrolopyridine derivatives for treating diseases are claimed, for example, in U.S. Pat. No. 6,232,320-B1, WO 2006/069080 and WO 2006/130160, and DE 29 09 754-A1 describes certain oxy-substituted benzofuran derivatives for treating atherosclerosis.

In contrast to the compounds of the prior art, the compounds claimed in the context of the present invention are characterized in that a disubstituted bicyclic heteroaryl core structure is attached at a certain spatial distance to a carboxylic acid or a carboxylic acid-like functionality.

The present invention provides compounds of the general formula (I)

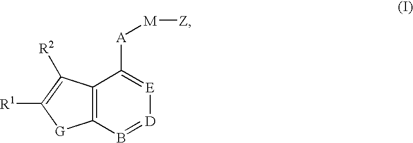

in which
B, D and E each represent CH or N,
G represents NH, O or S, with the proviso that
   G does not represent O if at the same time B and E represent N and D represents CH,
   and
   G does not represent NH or S if at the same time B, D and E represent CH,
A represents O or N—R³ in which
   R³ represents hydrogen, (C₁-C₆)-alkyl, (C₃-C₇)-cycloalkyl or (C₄-C₇)-cycloalkenyl,
M represents a group of the formula

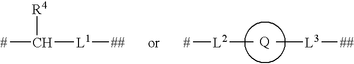

in which
# represents the point of attachment to the group A
and
## represents the point of attachment to the group Z,
R⁴ represents hydrogen or (C₁-C₄)-alkyl which may be substituted by hydroxyl or amino,
L¹ represents (C₁-C₇)-alkanediyl or (C₂-C₇)-alkenediyl which may be mono- or disubstituted by fluorine, or represents a group of the formula ♦-L^{1A}-V-L^{1B}-♦♦ in which
   ♦ represents the point of attachment to the group —CHR⁴,
   ♦♦ represents the point of attachment to the group Z,
   L^{1A} represents (C₁-C₅)-alkanediyl which may be mono- or disubstituted by identical or different substituents from the group consisting of (C₁-C₄)-alkyl and (C₁-C₄)-alkoxy,
   L^{1B} represents a bond or (C₁-C₃)-alkanediyl which may be mono- or disubstituted by fluorine,
   and
   V represents O or N—R⁵ in which
      R⁵ represents hydrogen, (C₁-C₆)-alkyl or (C₃-C₇)-cycloalkyl,
L² represents a bond or (C₁-C₄)-alkanediyl,
L³ represents (C₁-C₄)-alkanediyl which may be mono- or disubstituted by fluorine and in which a methylene group may be replaced by O or N—R⁶
   in which
   R⁶ represents hydrogen, (C₁-C₆)-alkyl or (C₃-C₇)-cycloalkyl,
   or represents (C₂-C₄)-alkenediyl,
and
Q represents (C₃-C₇)-cycloalkyl, (C₄-C₇)-cycloalkenyl, phenyl, 5- to 7-membered heterocyclyl or 5- or 6-membered heteroaryl, each of which may be substituted by up to two identical or different radicals selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino, where (C₁-C₄)-alkyl for its part may be substituted by hydroxyl, (C₁-C₄)-alkoxy, amino, mono- or di-(C₁-C₄)-alkylamino,
Z represents a group of the formula

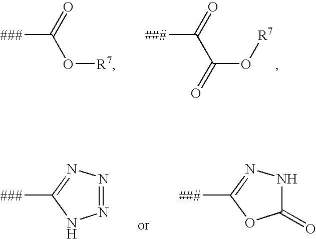

in which
### represents the point of attachment to the group L¹ or L³
and
R⁷ represents hydrogen or (C₁-C₄)-alkyl,
   and
R¹ and R² are identical or different and independently of one another represent (C₃-C₇)-cycloalkyl, (C₄-C₇)-cycloalkenyl, phenyl, 5- to 7-membered heterocyclyl or 5- or 6-membered heteroaryl each of which may be mono- to trisubstituted by identical or different radicals selected from the group consisting of halogen, cyano, nitro, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-acyl, amino, mono-(C₁-C₆)-alkylamino, di-(C₁-C₆)-alkylamino and (C₁-C₆)-acylamino,
   where (C₁-C₆)-alkyl and (C₁-C₆)-alkoxy for their part may each be substituted by cyano, hydroxyl, (C₁-C₄)-alkoxy, (C₁-C₄)-alkylthio, amino, mono- or di-(C₁-C₄)-alkylamino,
      or
R¹ and/or R² represent phenyl in which two radicals attached to adjacent ring carbon atoms together form a group of the formula —O—CH₂—O—, —O—CHF—O—, —O—CF₂—O—, —O—CH₂—CH₂—O— or —O—CF₂—CF₂—O—,
   and their salts, solvates and solvates of the salts.

Compounds according to the invention are the compounds of the formula (I) and the salts, solvates and solvates of the salts thereof, the compounds of the formulae below encompassed by the formula (I) and the salts, solvates and solvates of the salts thereof, and also the compounds encompassed by the formula (I) and mentioned below as working examples, and the salts, solvates and solvates of the salts thereof, provided the compounds encompassed by formula (I) and mentioned below are not already salts, solvates and solvates of the salts.

The compounds of the invention may, depending on their structure, exist in stereoisomeric forms (enantiomers, diastereomers). The present invention therefore relates to the enantiomers or diastereomers and respective mixtures thereof. The stereoisomerically pure constituents can be isolated in a known manner from such mixtures of enantiomers and/or diastereomers.

If the compounds of the invention may occur in tautomeric forms, the present invention encompasses all tautomeric forms.

Salts which are preferred for the purposes of the present invention are physiologically acceptable salts of the compounds of the invention. Also encompassed are salts which are themselves unsuitable for pharmaceutical uses but can be used for example for isolating or purifying the compounds of the invention.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, maleic acid, citric acid, fumaric acid, maleic acid and benzoic acid.

Physiologically acceptable salts of the compounds of the invention include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refers for the purposes of the invention to those forms of the compounds of the invention which form, in the solid or liquid state, a complex by coordination with solvent molecules. Hydrates are a specific form of solvates in which the coordination takes place with water. Hydrates are preferred solvates in the context of the present invention.

The present invention additionally encompasses the use of prodrugs of the compounds of the invention. The term “prodrugs” encompasses compounds which themselves may be biologically active or inactive, but are converted during their residence time in the body into compounds of the invention (for example by metabolism or hydrolysis).

In particular, for the compounds of the formula (I) in which

Z represents a group of the formula

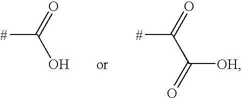

the present invention also includes hydrolyzable ester derivatives of these compounds. These are to be understood as meaning esters which can be hydrolyzed to the free carboxylic acids, as the compounds that are mainly active biologically, in physiologically media, under the conditions of the biological tests described later and in particular in vivo by enzymatic or chemical routes. (C₁-C₄)-alkyl esters, in which the alkyl group can be straight-chain or branched, are preferred as such esters. Particular preference is given to methyl or ethyl esters (see also the corresponding definitions of the radical R⁷).

In the context of the present invention, the substituents have the following meaning, unless specified otherwise:

(C₁-C₆)-Alkyl, (C₁-C₅)-alkyl, (C₁-C₄)-alkyl and (C₁-C₃)-alkyl stand in the context of the invention for a straight-chain or branched alkyl radical having respectively 1 to 6, 1 to 5, 1 to 4 and 1 to 3 carbon atoms. A straight-chain or branched alkyl radical having 1 to 4, in particular 1 to 3, carbon atoms is preferred. Examples which may be preferably mentioned are: methyl, ethyl, n-propyl, isopropyl, n-butyl, isobutyl, sec-butyl, tert-butyl, 1-ethylpropyl, n-pentyl and n-hexyl.

(C₂-C₆)-Alkenyl, (C₂-C₅)-alkenyl and (C₂-C₄)-alkenyl stand in the context of the invention for a straight-chain or branched alkenyl radical having respectively 2 to 6, 2 to 5 and 2 to 4 carbon atoms and one or two double bonds. A straight-chain or branched alkenyl radical having 2 to 4 carbon atoms and one double bond is preferred. Examples which may be preferably mentioned are: vinyl, allyl, isopropenyl and n-but-2-en-1-yl.

(C₂-C₄)-Alkynyl stands in the context of the invention for a straight-chain or branched alkynyl radical having 2 to 4 carbon atoms and one triple bond. A straight-chain alkynyl radical having 2 to 4 carbon atoms is preferred. Examples which may be preferably mentioned are: ethynyl, n-prop-1-in-1-yl, n-prop-2-in-1-yl, n-but-2-in-1-yl and n-but-3-in-1-yl.

(C₁-C₄)-Alkanediyl and (C₁-C₃)-alkanediyl stand in the context of the invention for a straight-chain or branched divalent alkyl radical having respectively 1 to 4 and 1 to 3 carbon atoms. In each case, a straight-chain alkanediyl radical having respectively 1 to 4 and 1 to 3 carbon atoms is preferred. Examples which may be preferably mentioned are: methylene, ethane-1,2-diyl(1,2-ethylene), ethane-1,1-diyl, propane-1,3-diyl(1,3-propylene), propane-1,1-diyl, propane-1,2-diyl, propane-2,2-diyl, butane-1,4-diyl(1,4-butylene), butane-1,2-diyl, butane-1,3-diyl and butane-2,3-diyl.

(C₁-C₇)-Alkanediyl, (C₁-C₅)-alkanediyl and (C₃-C₇)-alkanediyl stand in the context of the invention for a straight-chain or branched divalent alkyl radical having respectively 1 to 7, 1 to 5 and 3 to 7 carbon atoms. In each case, a straight-chain alkanediyl radical having respectively 1 to 7, 1 to 5 and 3 to 7 carbon atoms is preferred. Examples which may be preferably mentioned are: methylene, ethane-1,2-diyl(1,2-ethylene), ethane-1,1-diyl, propane-1,3-diyl(1,3-propylene), propane-1,1-diyl, propane-1,2-diyl, propane-2,2-diyl, butane-1,4-diyl(1,4-butylene), butane-1,2-diyl, butane-1,3-diyl, butane-2,3-diyl, pentane-1,5-diyl(1,5-pentylene), pentane-2,4-diyl, 3-methylpentane-2,4-diyl and hexane-1,6-diyl(1,6-hexylene).

(C₂-C₄)-Alkenediyl and (C₂-C₃)-alkenediyl stand in the context of the invention for a straight-chain or branched divalent alkenyl radical having respectively 2 to 4 and 2 to 3 carbon atoms and up to 2 double bonds. In each case, a straight-chain alkenediyl radical having respectively 2 to 4 and 2 to 3 carbon atoms and one double bond is preferred. Examples which may be preferably mentioned are: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl and buta-1,3-diene-1,4-diyl.

(C₂-C₇)-Alkenediyl and (C₃-C₇)-alkenediyl stand in the context of the invention for a straight-chain or branched divalent alkenyl radical having respectively 2 to 7 and 3 to 7 carbon atoms and up to 3 double bonds. In each case, a straight-chain alkenediyl radical having respectively 2 to 7 and 3 to 7 carbon atoms and one double bond is preferred. Examples which may be preferably mentioned are: ethene-1,1-diyl, ethene-1,2-diyl, propene-1,1-diyl, propene-1,2-diyl, propene-1,3-diyl, but-1-ene-1,4-diyl, but-1-ene-1,3-diyl, but-2-ene-1,4-diyl, buta-1,3-diene-1,4-diyl, pent-2-ene-1,5-diyl, hex-3-ene-1,6-diyl and hexa-2,4-diene-1,6-diyl.

(C₁-C₆)-Alkoxy and (C₁-C₄)-alkoxy stand in the context of the invention for a straight-chain or branched alkoxy radical having respectively 1 to 6 and 1 to 4 carbon atoms. A straight-chain or branched alkoxy radical having 1 to 4 carbon atoms is preferred. Examples which may be preferably mentioned are: methoxy, ethoxy, n-propoxy, isopropoxy, n-butoxy, tert-butoxy, n-pentoxy and n-hexoxy.

(C₁-C₆)-Alkylthio and (C₁-C₄)-alkylthio stand in the context of the invention for a straight-chain or branched alkylthio radical having respectively 1 to 6 and 1 to 4 carbon atoms. A straight-chain or branched alkylthio radical having 1 to 4 carbon atoms is preferred. Examples which may be preferably mentioned are: methylthio, ethylthio, n-propylthio, isopropylthio, n-butylthio, tert-butylthio, n-pentylthio and n-hexylthio.

(C₁-C₆)-Acyl [(C₁-C₆)-alkanoyl], (C₁-C₅)-acyl [(C₁-C₅)-alkanoyl] and (C₁-C₄)-acyl [(C₁-C₄)-alkanoyl] stand in the context of the invention for a straight-chain or branched alkyl radical having respectively 1 to 6, 1 to 5 and 1 to 4 carbon atoms which carries a doubly attached oxygen atom in the 1-position and is attached via the 1-position. A straight-chain or branched acyl radical having 1 to 4 carbon atoms is preferred. Examples which may be preferably mentioned are: formyl, acetyl, propionyl, n-butyryl, isobutyryl and pivaloyl.

Mono-(C₁-C₆)-alkylamino and mono-(C₁-C₄)-alkylamino stand in the context of the invention for an amino group having a straight-chain or branched alkyl substituent which has respectively 1 to 6 and 1 to 4 carbon atoms. A straight-chain or branched monoalkylamino radical having 1 to 4 carbon atoms is preferred. Examples which may be preferably mentioned are: methylamino, ethylamino, n-propylamino, isopropylamino and tert-butylamino.

Di-(C₁-C₆)-alkylamino and di-(C₁-C₄)-alkylamino stand in the context of the invention for an amino group having two identical or different straight-chain or branched alkyl substituents having respectively 1 to 6 and 1 to 4 carbon atoms. Straight-chain or branched dialkylamino radicals having in each case 1 to 4 carbon atoms are preferred. Examples which may be preferably mentioned are: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

(C₁-C₆)-Acylamino and (C₁-C₄)-acylamino stand in the context of the invention for an amino group having a straight-chain or branched acyl substituent which has respectively 1 to 6 and 1 to 4 carbon atoms and is attached via the carbonyl group. An acylamino radical having 1 to 4 carbon atoms is preferred. Examples which may be preferably mentioned are: formamido, acetamido, propionamido, n-butyramido and pivaloylamido.

(C₃-C₇)-Cycloalkyl, (C₃-C₆)-cycloalkyl and (C₄-C₆)-cycloalkyl stand in the context of the invention for a monocyclic saturated cycloalkyl group having respectively 3 to 7, 3 to 6 and 4 to 6 carbon atoms. A cycloalkyl radical having 3 to 6 carbon atoms is preferred. Examples which may be preferably mentioned are: cyclopropyl, cyclobutyl, cyclopentyl, cyclohexyl and cycloheptyl.

(C₄-C₇)-Cycloalkenyl, (C₄-C₆)-cycloalkenyl and (C₁-C₆)-cycloalkenyl stand in the context of the invention for a monocyclic cycloalkyl group having respectively 4 to 7, 4 to 6 and 5 or 6 carbon atoms and one double bond. A cycloalkenyl radical having 4 to 6, particularly preferably 5 or 6, carbon atoms is preferred. Examples which may be preferably mentioned are: cyclobutenyl, cyclopentenyl, cyclohexenyl and cycloheptenyl.

5- to 7-membered heterocyclyl stands in the context of the invention for a saturated or partially unsaturated heterocycle having 5 to 7 ring atoms which contains one or two ring heteroatoms from the group consisting of N and O and is attached via ring carbon atoms and/or, if appropriate, ring nitrogen atoms. A 5- or 6-membered saturated heterocycle having one or two ring heteroatoms from the group consisting of N and O is preferred. Examples which may be mentioned are: pyrrolidinyl, pyrrolinyl, pyrazolidinyl, tetrahydrofuranyl, piperidinyl, piperazinyl, dihydropyranyl, tetrahydropyranyl, morpholinyl, hexahydroazepinyl and hexahydro-1,4-diazepinyl. Preference is given to pyrrolidinyl, tetrahydrofuranyl, piperidinyl, piperazinyl, tetrahydropyranyl and morpholinyl.

5- or 6-membered heteroaryl stands in the context of the invention for an aromatic heterocycle (heteroaromatic) having 5 or 6 ring atoms which contains one or two ring heteroatoms from the group consisting of N, O and S and is attached via ring carbon atoms and/or, if appropriate, a ring nitrogen atom. Examples which may be mentioned are: furyl, pyrrolyl, thienyl, pyrazolyl, imidazolyl, thiazolyl, oxazolyl, isoxazolyl, isothiazolyl, pyridyl, pyrimidinyl, pyridazinyl and pyrazinyl. Preference is given to thienyl, pyridyl, pyrimidinyl, pyridazinyl and pyrazinyl.

Halogen includes in the context of the invention fluorine, chlorine, bromine and iodine. Preference is given to chlorine or fluorine.

If radicals in the compounds according to the invention are substituted, the radicals, unless specified otherwise, may be mono- or polysubstituted. In the context of the present invention, for all radicals that occur more than once, their meanings are independent of one another. Substitution by 1, 2 or 3 identical or different substituents is preferred. Very particular preference is given to substitution by one substituent.

In the context of the present invention, preference is given to compounds of the formula (I) in which the bicyclic ring system

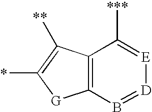

represents a heteroaryl group of the formula

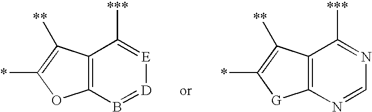

in which
* represents the point of attachment to the radical R¹,
** represents the point of attachment to the radical R²
and
*** represents the point of attachment to the group -A-M-Z,
B, D and E each represent CH or N, with the proviso that B does not represent N if at the same time D represents CH and E represents N,
and
G represents NH or S,
A represents O or N—R³ in which
   R³ represents hydrogen, (C₁-C₄)-alkyl or cyclopropyl,
M represents a group of the formula

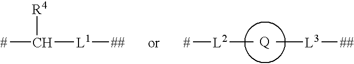

in which
# represents the point of attachment to the group A
and
## represents the point of attachment to the group Z,
R⁴ represents hydrogen or (C₁-C₃)-alkyl which may be substituted by hydroxyl or amino,
L¹ represents (C₃-C₇)-alkanediyl or (C₃-C₇)-alkenediyl which may be mono- or disubstituted by fluorine, or a group of the formula ♦-L^{1A}-V-L^{1B}-♦♦ in which
   ♦ represents the point of attachment to the group —CHR⁴,
   ♦♦ represents the point of attachment to the group Z,
   L^{1A} represents (C₁-C₃)-alkanediyl which may be mono- or disubstituted by identical or different substituents from the group consisting of methyl and ethyl,
   L^{1B} represents (C₁-C₃)-alkanediyl which may be mono- or disubstituted by fluorine,
   and
   V represents O or N—R⁵ in which
      R⁵ represents hydrogen, (C₁-C₃)-alkyl or cyclopropyl,
L² represents a bond or (C₁-C₃)-alkanediyl,
L³ represents (C₁-C₃)-alkanediyl which may be mono- or disubstituted by fluorine, (C₂-C₃)-alkenediyl or a group of the formula —W—CR⁸R⁹—, —W—CH₂—CR⁸R⁹— or —CH₂—W—CR⁸R⁹— in which
   represents the point of attachment to the ring Q,
   represents the point of attachment to the group Z,
   W represents O or N—R⁶ in which
      R⁶ represents hydrogen, (C₁-C₃)-alkyl or cyclopropyl,
   and
   R⁸ and R⁹ independently of one another represent hydrogen or fluorine,
and
Q represents (C₄-C₆)-cycloalkyl, (C₄-C₆)-cycloalkenyl, phenyl or 5- or 6-membered heterocyclyl, each of which may be up substituted by up to two identical or different radicals selected from the group consisting of fluorine, chlorine, (C₁-C₃)-alkyl, trifluoromethyl, hydroxyl, methoxy, ethoxy, trifluoromethoxy, amino, methylamino, ethylamino, dimethylamino and diethylamino,
Z represents a group of the formula

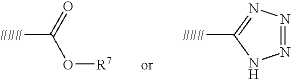

in which
### represents the point of attachment to the group L¹ or L³
and
R⁷ represents hydrogen, methyl or ethyl,
   and
R¹ and R² are identical or different and independently of one another represent (C₄-C₆)-cycloalkenyl, phenyl or 5- or 6-membered heteroaryl, each of which may be mono- or disubstituted by identical or different radicals selected from the group consisting of fluorine, chlorine, cyano, (C₁-C₅)-alkyl, (C₂-C₅)-alkenyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₅)-acyl, amino, mono-(C₁-C₄)-alkyl-amino, di-(C₁-C₄)-alkylamino and (C₁-C₄)-acylamino,
   or
R¹ and/or R² represent phenyl in which two radicals attached to adjacent ring carbon atoms together form a group of the formula —O—CH₂—O—, —O—CHF—O— or —O—CF₂—O—,
   and to their salts, solvates and solvates of the salts.

In the context of the present invention, particular preference is given to compounds of the formula (I) in which the bicyclic ring system

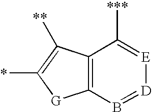

represents a heteroaryl group of the formula

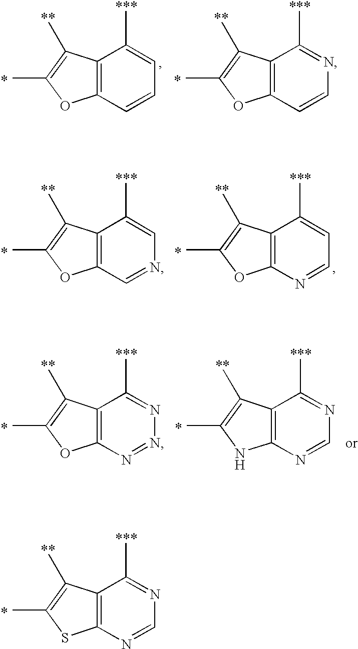

in which
* represents the point of attachment to the radical R¹,
** represents the point of attachment to the radical R²
and
*** represents the point of attachment to the group -A-M-Z,
A represents O or NH,
M represents a group of the formula

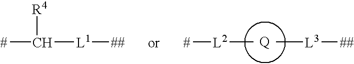

in which
# represents the point of attachment to the group A
and
## represents the point of attachment to the group Z,
R⁴ represents hydrogen, methyl or ethyl,
L¹ represents (C₃-C₇)-alkanediyl, (C₃-C₇)-alkenediyl or a group of the formula ♦-L^{1A}-V-L^{1B}-♦♦ in which
   ♦ represents the point of attachment to the group —CHR⁴,
   ♦♦ represents the point of attachment to the group Z,
   L^{1A} represents (C₁-C₃)-alkanediyl which may be mono- or disubstituted by methyl,
   L^{1B} represents (C₁-C₃)-alkanediyl
   and
   V represents O or N—CH₃,
L² represents a bond, methylene, ethane-1,1-diyl or ethane-1,2-diyl,
L³ represents (C₁-C₃)-alkanediyl or a group of the formula —W—CH₂— or —W—CH₂—CH₂— in which
   represents the point of attachment to the ring Q,
   represents the point of attachment to the group Z
   and
   W represents O or N—R⁶ in which
      R⁶ represents hydrogen or (C₁-C₃)-alkyl,
and
Q represents cyclobutyl, cyclopentyl, cyclopentenyl, cyclohexyl, pyrrolidinyl, piperidinyl, tetrahydrofuranyl, tetrahydropyranyl, morpholinyl or phenyl, each of which may be substituted by up to two identical or different radicals selected from the group consisting of fluorine, methyl, ethyl, trifluoromethyl, hydroxyl, methoxy and ethoxy,
Z represents a group of the formula

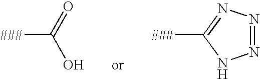

in which
### represents the point of attachment to the group L¹ or L³,
   and
R¹ and R² are identical or different and independently of one another represent cyclopenten-1-yl, cyclohexen-1-yl, phenyl, thienyl or pyridyl, each of which may be mono- or disubstituted by identical or different radicals selected from the group consisting of fluorine, chlorine, cyano, (C₁-C₄)-alkyl, (C₂-C₄)-alkenyl, (C₁-C₄)-alkoxy, trifluoromethyl and trifluoromethoxy,
   and to their salts, solvates and solvates of the salts.

In the context of the present invention, special preference is given to compounds of the formula (I-A)

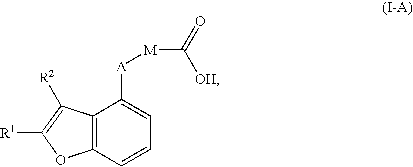

in which
A represents O or NH,
M represents a group of the formula

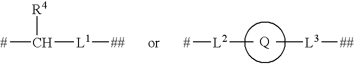

in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
   ♦ represents the point of attachment to the group —CHR⁴,
   ♦♦ represents the point of attachment to the carboxylic acid grouping,
   L^{1A} represents methylene or ethane-1,2-diyl which may be mono- or disubstituted by methyl,
   and
   L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
   represents the point of attachment to the ring Q
   and
   represents the point of attachment to the carboxylic acid grouping,
and
Q represents cyclopentyl, cyclohexyl or phenyl,
R¹ represents phenyl which may be substituted by fluorine or chlorine,
   and
R² represents phenyl which may be substituted by methyl, ethyl, methoxy or ethoxy,
   and to their salts, solvates and solvates of the salts.

Special preference is also given to compounds of the formula (I-B)

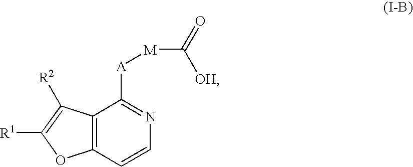

in which
A represents O or NH,
M represents a group of the formula

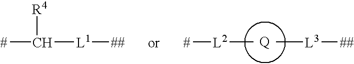

in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
   ♦ represents the point of attachment to the group —CHR⁴,
   ♦♦ represents the point of attachment to the carboxylic acid grouping,
   L^{1A} represents methylene or ethane-1,2-diyl which may be mono- or disubstituted by methyl,
   and
   L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
   represents the point of attachment to the ring Q
   and
   represents the point of attachment to the carboxylic acid grouping,
and
Q represents cyclopentyl, cyclohexyl or phenyl,
R¹ represents phenyl which may be substituted by fluorine or chlorine,
   and
R² represents phenyl which may be substituted by methyl, ethyl, methoxy or ethoxy,
   and to their salts, solvates and solvates of the salts.

Special preference is also given to compounds of the formula (I-C)

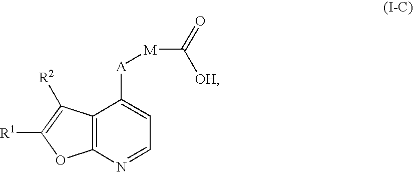

in which
A represents O or NH,
M represents a group of the formula

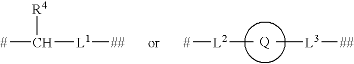

in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
   ♦ represents the point of attachment to the group —CHR⁴,
   ♦♦ represents the point of attachment to the carboxylic acid grouping,
   L^{1A} represents methylene or ethane-1,2-diyl which may be mono- or disubstituted by methyl,
   and
   L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
   represents the point of attachment to the ring Q
   and
   represents the point of attachment to the carboxylic acid grouping,
and
Q represents cyclopentyl, cyclohexyl or phenyl,
R¹ represents phenyl which may be substituted by fluorine or chlorine,
   and
R² represents phenyl which may be substituted by methyl, ethyl, methoxy or ethoxy,
   and to their salts, solvates and solvates of the salts.

Special preference is also given to compounds of the formula (I-D)

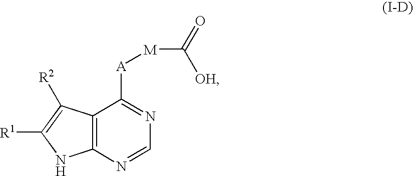

in which
A represents O or NH,
M represents a group of the formula

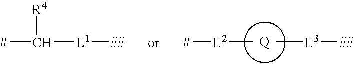

in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
   ♦ represents the point of attachment to the group —CHR⁴,
   ♦♦ represents the point of attachment to the carboxylic acid grouping,
   L^{1A} represents methylene or ethane-1,2-diyl which may be mono- or disubstituted by methyl,
   and
   L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
   represents the point of attachment to the ring Q
   and
   represents the point of attachment to the carboxylic acid grouping,
and
Q represents cyclopentyl, cyclohexyl or phenyl,
R¹ represents phenyl which may be substituted by fluorine or chlorine,
   and
R² represents phenyl which may be substituted by methyl, ethyl, methoxy or ethoxy,
   and to their salts, solvates and solvates of the salts.

Special preference is also given to compounds of the formula (I-E)

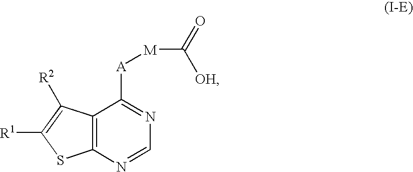

in which
A represents O or NH,
M represents a group of the formula

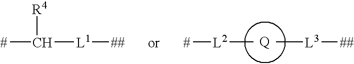

in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
   ♦ represents the point of attachment to the group —CHR⁴,
   ♦♦ represents the point of attachment to the carboxylic acid grouping,
   L^{1A} represents methylene or ethane-1,2-diyl which may be mono- or disubstituted by methyl,
   and
   L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
   represents the point of attachment to the ring Q
   and
   represents the point of attachment to the carboxylic acid grouping,
and
Q represents cyclopentyl, cyclohexyl or phenyl,
R¹ represents phenyl which may be substituted by fluorine or chlorine,
   and
R² represents phenyl which may be substituted by methyl, ethyl, methoxy or ethoxy,
   and to their salts, solvates and solvates of the salts.

The individual definitions of radicals given in the respective combinations and preferred combinations of radicals are, independently of the given combination of radicals in question, also replaced by radical definitions of other combinations.

Particular preference is given to combinations of two or more of the preferred ranges mentioned above.

In the context of the present invention, very particular preference is given to the compounds mentioned below:
(6R)-6-{[5-(4-methoxyphenyl)-6-phenyl-7H-pyrrolo[2,3-d]pyrimidin-4-yl]oxy}heptanoic acid;
(6R)-6-{[5-(4-methoxyphenyl)-6-phenylthieno[2,3-d]pyrimidin-4-yl]oxy}heptanoic acid;
(6R)-6-{[5-(4-ethylphenyl)-6-phenylthieno[2,3-d]pyrimidin-4-yl]oxy}heptanoic acid;
(6R)-6-{[3-(4-methoxyphenyl)-2-phenyl-1-benzofuran-4-yl]oxy}heptanoic acid;
(3-{[3-(4-methoxyphenyl)-2-phenylfuro[3,2-c]pyridin-4-yl]amino}-2,2-dimethylpropoxy)acetic acid;
(6R)-6-{[3-(4-methoxyphenyl)-2-phenylfuro[3,2-c]pyridin-4-yl]oxy}heptanoic acid;
3-(3-{[3-(4-ethylphenyl)-2-phenylfuro[2,3-b]pyridin-4-yl]amino}-2,2-dimethylpropoxy)-propanoic acid;
6-{[3-(4-ethylphenyl)-2-phenylfuro[2,3-b]pyridin-4-yl]amino}hexanoic acid;
(3-{[3-(4-ethylphenyl)-2-phenylfuro[2,3-b]pyridin-4-yl]oxy}propoxy)acetic acid;
   and
(6R)-6-{[3-(4-ethylphenyl)-2-phenylfuro[2,3-b]pyridin-4-yl]oxy}heptanoic acid,
   and to their salts, solvates and solvates of the salts.

The invention furthermore provides a process for preparing the compounds of the formula (I) according to the invention in which Z represents —COOH, characterized in that either

[A] compounds of the formula (II)

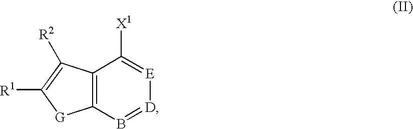

in which B, D, E, G, R¹ and R² each have the meanings given above
and
X¹ represents a leaving group such as, for example, halogen, in particular chlorine,
are reacted in an inert solvent in the presence of a base with a compound of the formula (III)

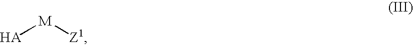

in which A and M have the meanings given above
and
Z¹ represents cyano or a group of the formula —[C(O)]_{y}—COOR^{7A} in which
   y represents the number 0 or 1
   and
   R^{7A} represents (C₁-C₄)-alkyl,
to give compounds of the formula (IV)

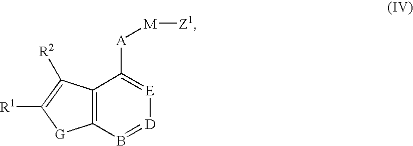

in which A, B, D, E, G, M, Z¹, R¹ and R² each have the meanings given above,
   or
   [B] compounds of the formula (V)

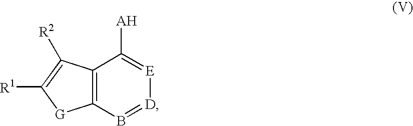

in which A, B, D, E, G, R¹ and R² each have the meanings given above, are reacted in an inert solvent in the presence of a base with a compound of the formula (VI)

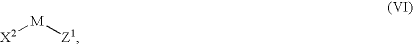

in which M and Z¹ have the meanings given above
and
X² represents a leaving group such as, for example, halogen, mesylate, tosylate or triflate,
to give compounds of the formula (IV)

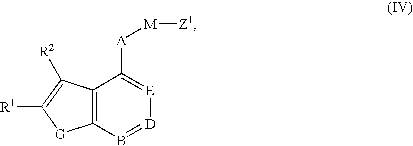

in which A, B, D, E, G, M, Z¹, R¹ and R² each have the meanings given above,
   and the compounds of the formula (IV) are then converted by hydrolysis of the ester or cyano group Z¹ into the carboxylic acids of the formula (Ia)

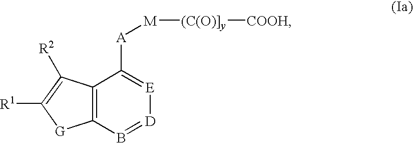

in which A, B, D, E, G, M, R¹, R² and y each have the meanings given above, and these are, if appropriate, converted into their solvates, salts and/or solvates of the salts using the appropriate (i) solvents and/or (ii) bases or acids.

Inert solvents for process steps (II)+(III)→(IV) and (V)+(VI)→(IV) are, for example, ethers, such as diethyl ether, methyl tert-butyl ether, dioxane, tetrahydro-furan, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethane, tetrachloroethane, trichloro-ethylene, chlorobenzene or chlorotoluene, or other solvents, such as dimethyl-formamide (DMF), dimethyl sulfoxide (DMSO), N,N′-dimethylpropyleneurea (DMPU), N-methylpyrrolidone (NMP) or acetonitrile. It is also possible to use mixtures of the solvents mentioned. Preference is given to using tetrahydrofuran, toluene, dimethylformamide, dimethyl sulfoxide or mixtures of these solvents.

However, if appropriate, the process steps (II)+(III)→(IV) and (V)+(VI)→(IV) can also be carried out in the absence of a solvent.

Suitable bases for the process steps (II)+(III)→(IV) and (V)+(VI)→(IV) are customary inorganic or organic bases. These preferably include alkali metal hydroxides, such as, for example, lithium hydroxide, sodium hydroxide, or potassium hydroxide, alkali metal or alkaline earth metal carbonates, such as lithium carbonate, sodium carbonate, potassium carbonate, calcium carbonate or cesium carbonate, alkali metal alkoxides, such as sodium tert-butoxide or potassium tert-butoxide, alkali metal hydrides, such as sodium hydride or potassium hydride, amides, such as lithium bis(trimethylsilyl)amide or potassium bis(trimethylsilyl)amide or lithium diisopropylamide, organic metallic compounds, such as butyllithium or phenyllithium, or organic amines, such as triethylamine, N-methylmorpholine, N-methylpiperidine, N,N-diisopropylethylamine or pyridine.

In the case of the reaction with alcohol derivatives [A in (III) and (V)=O], phosphazene bases (so-called “Schwesinger bases”), such as, for example, P2-t-Bu or P4-t-Bu are likewise expedient [cf., for example, R. Schwesinger, H. Schlemper, Angew. Chem. Int. Ed. Engl. 26, 1167 (1987); T. Pietzonka, D. Seebach, Chem. Ber. 124, 1837 (1991)].

In the reaction with amine derivatives [A in (III) and (V)=N], the base used is preferably a tertiary amine, such as, in particular, N,N-diisopropylethylamine or sodium tert-butoxide. However, if appropriate, these reactions can—if an excess of the amine component (III) is used—also be carried out without the addition of an auxiliary base. In the reaction with alcohol derivatives [A in (III)=O], preference is given to sodium hydride, potassium carbonate or cesium carbonate or the phosphazene bases P2-t-Bu and P4-t-Bu.

If appropriate, the process steps (II)+(III)→(IV) and (V)+(VI)→(IV) can advantageously be carried out with addition of a crown ether.

In one process variant, the reactions (II)+(III)→(IV) and (V)+(VI)→(IV) can also be carried out in a two-phase mixture consisting of an aqueous alkali metal hydroxide solution as base and one of the hydrocarbons or halogenated hydrocarbons mentioned above as further solvent, using a phase-transfer catalyst, such as tetrabutylammonium hydrogen sulfate or tetrabutylammonium bromide.

The process steps (II)+(III)→(IV) and (V)+(VI)→(IV) are, in the reaction with amine derivatives [A in (III) and (V)=N], generally carried out in a temperature range of from +50° C. to +200° C., preferably at from +80° C. to +150° C. In the reaction with alcohol derivatives [A in (III) and (V)=O], the reactions are generally carried out in a temperature range of from −20° C. to +120° C., preferably at from 0° C. to +80° C. The hydrolysis of the ester or nitrile group Z¹ in process step (IV)—(Ia) is carried out by customary methods by treating the esters or nitriles in inert solvents with acids or bases, where in the latter case the salts initially formed are converted by treatment with acid into the free carboxylic acids. In the case of the tert-butyl esters, the ester cleavage is preferably carried out using acids.

Suitable inert solvents for these reactions are water or the organic solvents customary for ester cleavage. These preferably include alcohols, such as methanol, ethanol, n-propanol, isopropanol, n-butanol or tert-butanol, or ethers, such as diethyl ether, tetrahydrofuran, dioxane or glycol dimethyl ether, or other solvents, such as acetone, dichloromethane, dimethylformamide or dimethyl sulfoxide. It is also possible to use mixtures of the solvents mentioned. In the case of a basic ester hydrolysis, preference is given to using mixtures of water with dioxane, tetrahydrofuran, methanol and/or ethanol, and for nitrile hydrolysis, preference is given to using water and/or n-propanol. In the case of the reaction with trifluoroacetic acid, preference is given to using dichloromethane, and in the case of the reaction with hydrogen chloride, preference is given to using tetrahydrofuran, diethyl ether, dioxane or water.

Suitable bases are the customary inorganic bases. These preferably include alkali metal hydroxides or alkaline earth metal hydroxides, such as, for example, sodium hydroxide, lithium hydroxide, potassium hydroxide or barium hydroxide, or alkali metal carbonates or alkaline earth metal carbonates, such as sodium carbonate, potassium carbonate or calcium carbonate. Particular preference is given to sodium hydroxide or lithium hydroxide.

Acids suitable for the ester cleavage are, in general, sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid, or mixtures thereof, if appropriate with added water. Preference is given to hydrogen chloride or trifluoroacetic acid in the case of the tert-butyl esters and to hydrochloric acid in the case of the methyl esters.

The ester cleavage is generally carried out in a temperature range of from 0° C. to +100° C., preferably at from +0° C. to +50° C. The nitrile hydrolysis is generally carried out in a temperature range of from +50° C. to +150° C., preferably at from +80° C. to +120° C.

Reactions mentioned can be carried out at atmospheric, elevated or reduced pressure (for example from 0.5 to 5 bar). In general, reactions are carried out at atmospheric pressure.

The compounds of the formula (I) according to the invention in which Z represents a group of the formula

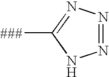

can be prepared by reacting compounds of the formula (IV) in which Z¹ represents cyano in an inert solvent with an alkali metal azide in the presence of ammonium chloride or with trimethylsilyl azide, if appropriate in the presence of a catalyst.

Inert solvents for this reaction are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, or other solvents, such as dimethyl sulfoxide, dimethylformamide, N,N′-dimethylpropyleneurea (DMPU) or N-methylpyrrolidone (NMP). It is also possible to use mixtures of the solvents mentioned. Preference is given to using toluene.

A suitable azide reagent is in particular sodium azide in the presence of ammonium chloride or trimethylsilyl azide. The latter reaction can advantageously be carried out in the presence of a catalyst. Suitable for this purpose are in particular compounds such as di-n-butyltin oxide, trimethylaluminum or zinc bromide. Preference is given to using trimethylsilyl azide in combination with di-n-butyltin oxide.

The reaction is generally carried out in a temperature range of from +50° C. to +150° C., preferably at from +60° C. to +110° C. The reaction can be carried out at atmospheric, elevated or reduced pressure (for example from 0.5 to 5 bar). In general, the reaction is carried out at atmospheric pressure.

The compounds of the formula (I) according to the invention in which Z represents a group of the formula

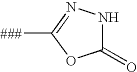

can be prepared by converting compounds of the formula (IV) in which Z¹ represents methoxycarbonyl or ethoxycarbonyl [y=0] initially in an inert solvent with hydrazine into compounds of the formula (VII)

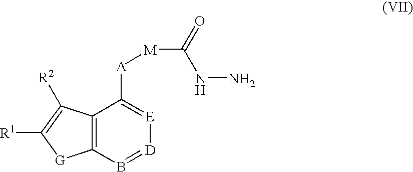

in which A, B, D, E, G, M, R¹ and R² each have the meanings given above, and then in an inert solvent with phosgene or a phosgene equivalent, such as, for example, N,N′-carbonyl diimidazole.

Suitable inert solvents for the first step of this reaction sequence are in particular alcohols, such as methanol, ethanol, n-propanol, isopropanol, n-butanol or tert-butanol, or ethers, such as diethyl ether, dioxane, tetrahydrofuran, glycol dimethyl ether or diethylene glycol dimethyl ether. It is also possible to use mixtures of these solvents. Preference is given to using a mixture of methanol and tetrahydrofuran. The second reaction step is preferably carried out in an ether, in particular in tetrahydrofuran. The reactions are generally carried out in a temperature range of from 0° C. to +70° C., under atmospheric pressure.

The compounds of the formula (I) according to the invention in which L¹ represents a group of the formula ♦-L^{1A}-V-L^{1B}-♦♦ in which L^{1A}, L^{1B} and V have the meanings given above can alternatively also be prepared by converting compounds of the formula (VIII)

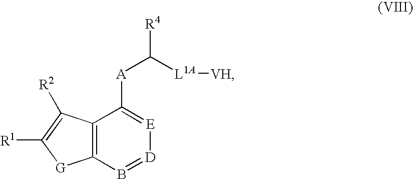

in which A, B, D, E, G, L^{1A}, V, R¹, R² and R⁴ each have the meanings given above
in the presence of a base, if appropriate in an inert solvent, with a compound of the formula (IX)

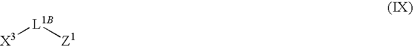

in which L^{1B} and Z¹ have the meanings given above
and
X³ represents a leaving group, such as, for example, halogen, mesylate, tosylate or triflate,
or, in the case that L^{1B} represents —CH₂CH₂— with a compound of the formula (X)

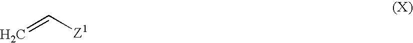

in which Z¹ has the meaning given above,
into compounds of the formula (IV-A)

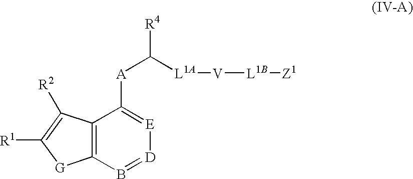

in which A, B, D, E, G, L^{1A}, L^{1B}, V, Z¹, R¹, R² and R⁴ each have the meanings given above,
and then reacting these further, in a manner corresponding to the process described above.

The compounds of the formula (VIII) can—analogously to the preparation of the compounds (IV)—be obtained by base-catalyzed reaction of a compound of the formula (II) or (V) with a compound of the formula (XI) or (XII)

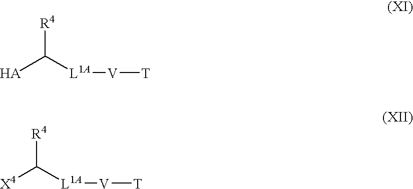

in which A, L^{1A}, V and R⁴ each have the meanings given above,
T represents hydrogen or a temporary O- or N-protective group
and
X⁴ represents a leaving group, such as, for example, halogen, mesylate, tosylate or triflate,
(cf. also Reaction Schemes 1 and 2 below).

In an analogous manner, the compounds of the formula (I) according to the invention in which L³ represents a group of the formula —W—CR⁸R⁹— or —W—CH₂—CR⁸R⁹— in which W, R⁸ and R⁹ have the meanings given above can also be prepared by converting compounds of the formula (XIII)

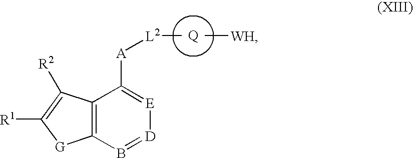

in which A, B, D, E, G, L², Q, W, R¹ and R² each have the meanings given above,
in the presence of a base, if appropriate in an inert solvent, with a compound of the formula (XIV)

X⁵—(CH₂)ₘ—CR⁸R⁹—Z¹ (XIV),

in which R⁸, R⁹ and Z¹ each have the meanings given above,
m represents the number 0 or 1
and
X⁵ represents a leaving group, such as, for example, halogen, mesylate, tosylate or triflate,
or in the case that L³ represents —W—CH₂CH₂— with a compound of the formula (X)

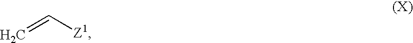

in which Z¹ has the meaning given above,
into compounds of the formula (IV-B)

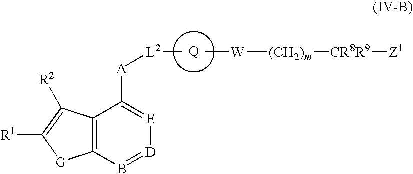

in which A, B, D, E, G, L², Q, W, Z¹, R¹, R², R⁸, R⁹ and m each have the meanings given above,
and then reacting these further according to one of the processes described above.

The compounds of the formula (XIII) can—analogously to the preparation of the compounds (IV)—be obtained by base-catalyzed reaction of a compound of the formula (II) or (V) with a compound of the formula (XV) or (XVI)

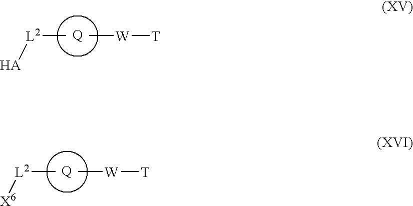

in which A, L², Q and W each have the meanings given above,
T represents hydrogen or a temporary O- or N-protective group
and
X⁶ represents a leaving group, such as, for example, halogen, mesylate, tosylate or triflate,
(cf. also Reaction Schemes 1 and 2 below).

For the process steps (VIII)+(IX) and (X)→(IV-A), (II)+(XI)→(VIII), (V)+(XII)→(VIII), (XIII)+(XIV) and (X)→(IV-B), (II)+(XV)→(XIII) and (V)+(XVI)→(XIII), the reaction parameters described above for the reactions (II)+(III)→(IV) and (V)+(VI)→(IV), such as solvents, bases and reaction temperatures, are used in an analogous manner.

The compounds of the formulae (II) and (V) are known from the literature or can be prepared analogously to methods described in the literature (see also Reaction schemes 3-10 below and the literature cited therein).

The compounds of the formulae (III), (VI), (IX), (X), (XI), (XII), (XIV), (XV) and (XVI) are commercially available, known from the literature or can be prepared analogously to processes known from the literature.

The preparation of the compounds according to the invention can be illustrated by way of example by the synthesis schemes below:

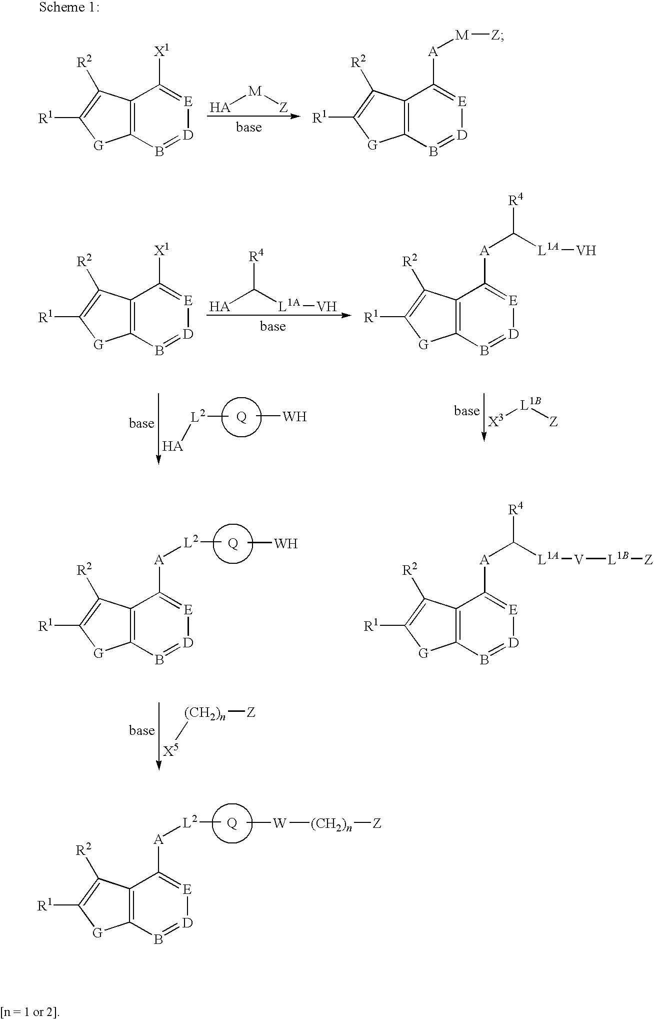

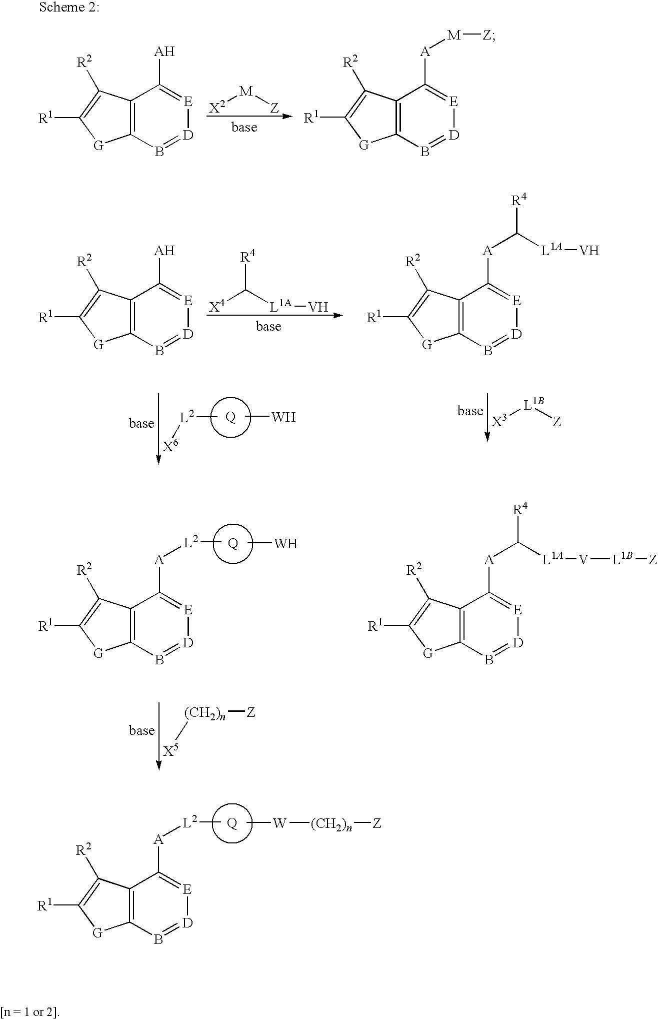

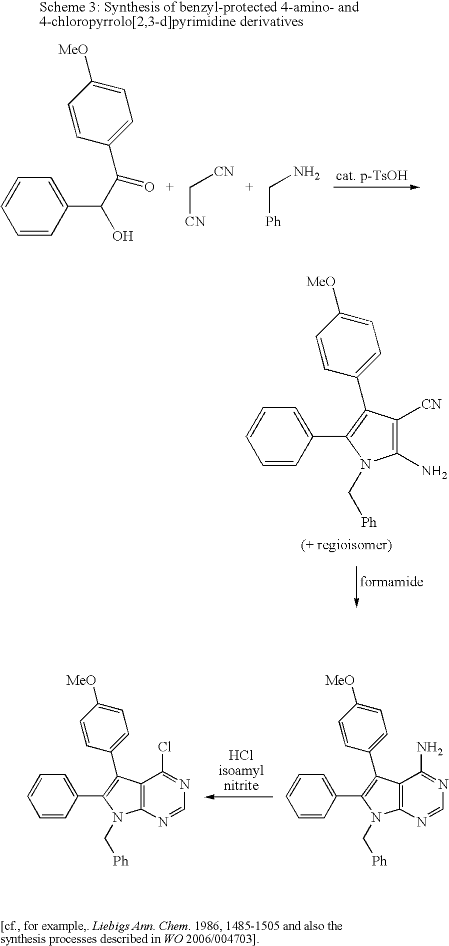

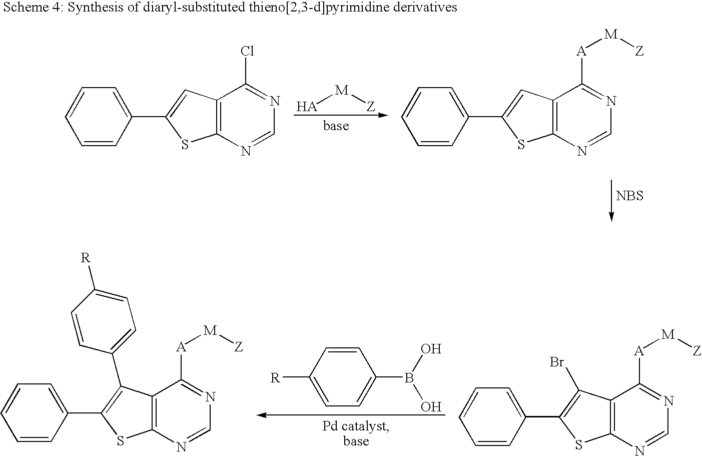

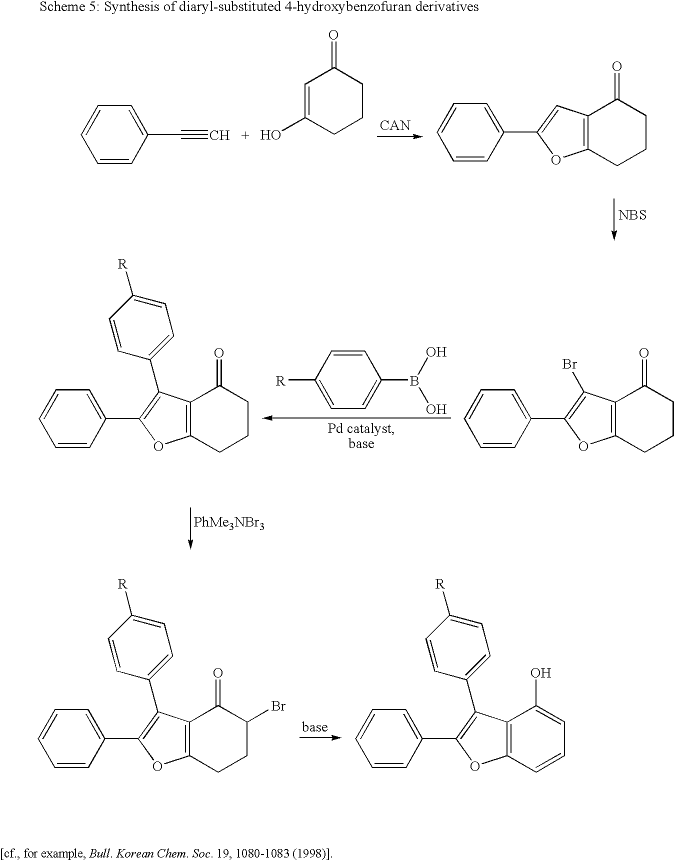

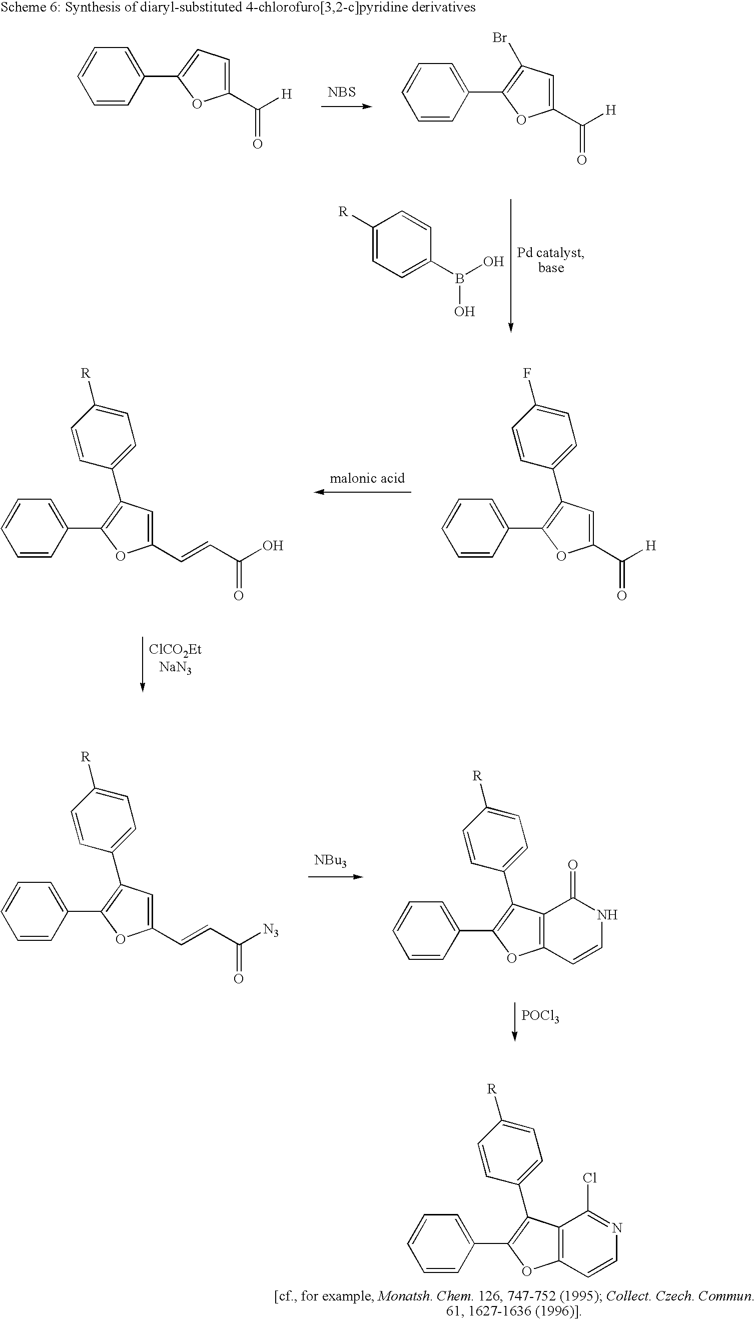

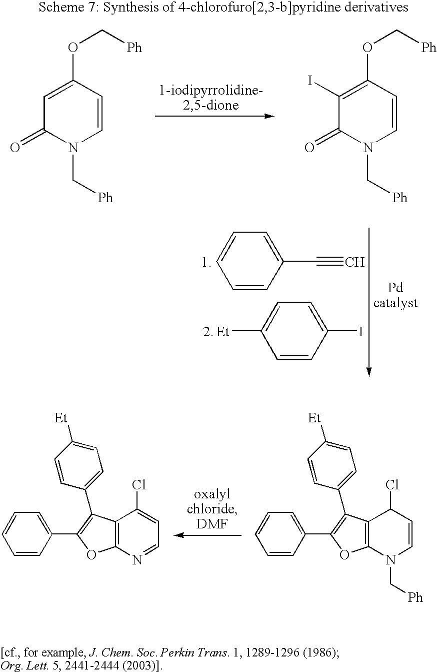

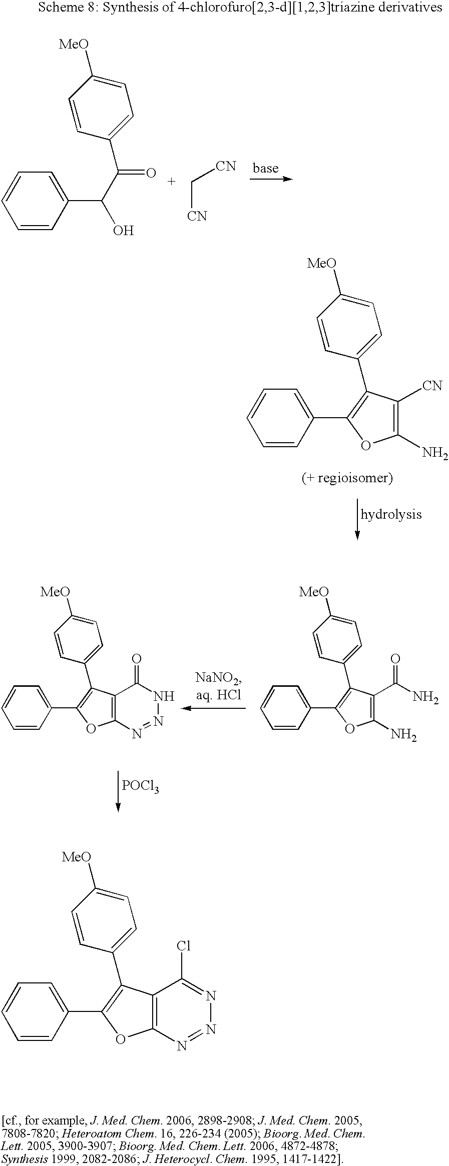

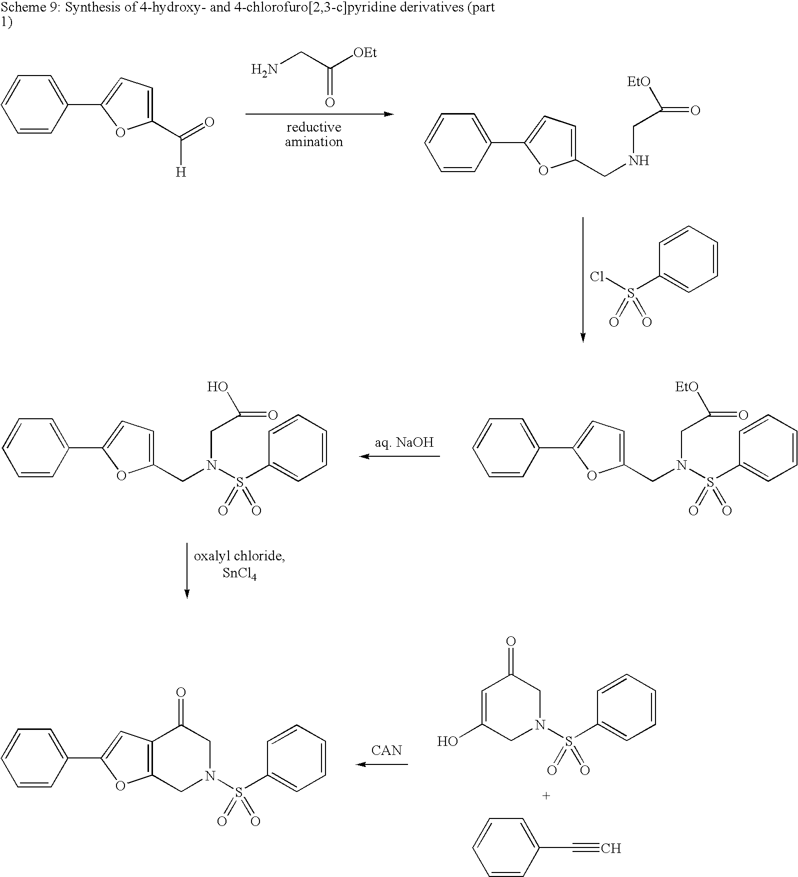

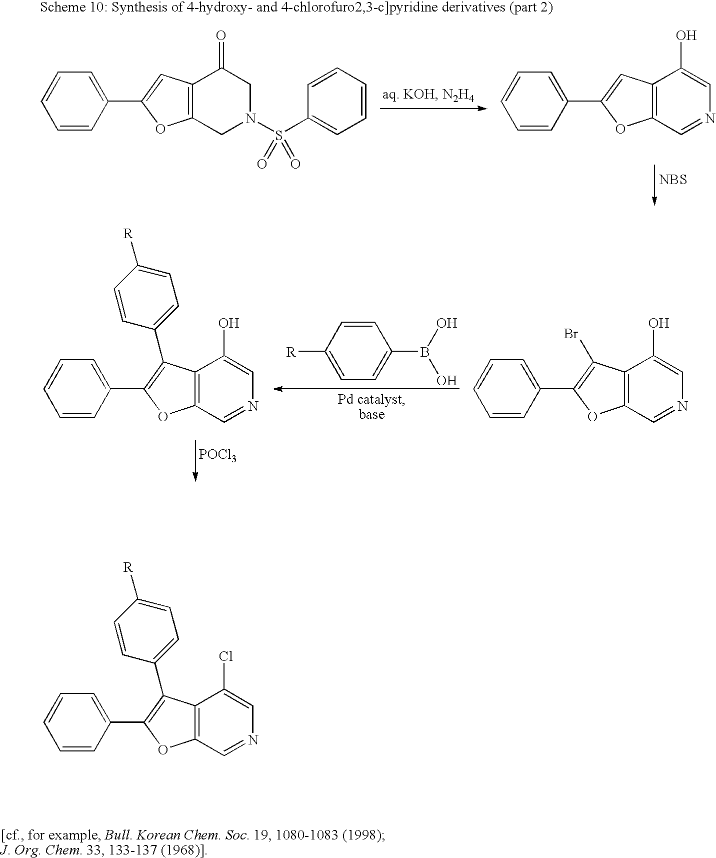

The compounds according to the invention possess valuable pharmacological properties and can be used for the prevention and treatment of diseases in humans and animals. The compounds according to the invention are chemically and metabolically stabile, non-prostanoid activators of the IP receptor.

They are thus suitable in particular for the prophylaxis and/or treatment of cardiovascular diseases such as stable and unstable angina pectoris, of hypertension and heart failure, pulmonary hypertension, for the prophylaxis and/or treatment of thromboembolic diseases and ischaemias such as myocardial infarction, stroke, transient and ischaemic attacks and subarachnoid haemorrhage, and for the prevention of restenosis such as after thrombolytic treatments, percutaneous transluminal angioplasty (PTA), coronary angioplasty (PTCA) and bypass surgery.

The compounds according to the invention are particularly suitable for the treatment and/or prophylaxis of pulmonary hypertension (PH) including its various manifestations. The compounds of the invention are therefore particularly suitable for the treatment and/or prophylaxis of pulmonary arterial hypertension (PAH) and its subtypes such as idiopathic and familial pulmonary arterial hypertension, and the pulmonary arterial hypertension which is associated for example with portal hypertension, fibrotic disorders, HIV infection or inappropriate medications or toxins.

The compounds of the invention can also be used for the treatment and/or prophylaxis of other types of pulmonary hypertension. Thus, for example, they can be employed for the treatment and/or prophylaxis of pulmonary hypertension associated with left atrial or left ventricular disorders and with left heart valve disorders. In addition, the compounds of the invention are suitable for the treatment and/or prophylaxis of pulmonary hypertension associated with chronic obstructive pulmonary disease, interstitial pulmonary disease, pulmonary fibrosis, sleep apnoea syndrome, disorders with alveolar hypoventilation, altitude sickness and pulmonary development impairments.

The compounds of the invention are furthermore suitable for the treatment and/or prophylaxis of pulmonary hypertension based on chronic thrombotic and/or embolic disorders such as, for example, thromboembolism of the proximal pulmonary arteries, obstruction of the distal pulmonary arteries and pulmonary embolism. The compounds of the invention can further be used for the treatment and/or prophylaxis of pulmonary hypertension connected with sarcoidosis, histiocytosis X or lymphangioleiomyomatosis, and where the pulmonary hypertension is caused by external compression of vessels (lymph nodes, tumor, fibrosing mediastinitis).

In addition, the compounds according to the invention can also be used for the treatment and/or prophylaxis of peripheral and cardial vascular diseases, peripheral occlusive diseases (PAOD, PVD) and disturbances of peripheral blood flow.

Furthermore, the compounds according to the invention can be used for the treatment of arteriosclerosis, hepatitis, asthmatic diseases, chronic obstructive pulmonary diseases (COPD), pulmonary edema, fibrosing lung diseases such as idiopathic pulmonary fibrosis (IPF) and ARDS, inflammatory vascular diseases such as scleroderma and lupus erythematosus, renal failure, arthritis and osteoporosis, and also for the prophylaxis and/or treatment of cancers, especially of metastasizing tumors.

Moreover, the compounds according to the invention can also be used as an addition to the preserving medium of an organ transplant, e.g. kidneys, lungs, heart or islet cells.

The present invention further relates to the use of the compounds according to the invention for the treatment and/or prophylaxis of diseases, and especially of the aforementioned diseases.

The present invention further relates to the use of the compounds according to the invention for the production of a medicinal product for the treatment and/or prophylaxis of diseases, and especially of the aforementioned diseases.

The present invention further relates to a method for the treatment and/or prophylaxis of diseases, especially of the aforementioned diseases, using an effective amount of at least one of the compounds according to the invention.

The compounds of the invention can be employed alone or, if required, in combination with other active ingredients. The present invention further relates to medicaments comprising at least one of the compounds of the invention and one or more further active ingredients, especially for the treatment and/or prophylaxis of the aforementioned disorders. Suitable active ingredients for combinations are by way of example and preferably:
organic nitrates and NO donors such as, for example, sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil;
NO-independent but heme-dependent stimulators of guanylate cyclase such as in particular the compounds described in WO 00/06568, WO 00/06569, WO 02/42301 and WO 03/095451;
NO— and heme-independent activators of guanylate cyclase, such as in particular the compounds described in WO 01/19355, WO 01/19776, WO 01/19778, WO 01/19780, WO 02/070462 and WO 02/070510;
compounds which inhibit human neutrophile elastase (HNE), such as, for example, sivelestat, DX-890 (Reltran), elafin or in particular the compounds described in WO 03/053930, WO 2004/020410, WO 2004/020412, WO 2004/024700, WO 2004/024701, WO 2005/080372, WO 2005/082863 and WO 2005/082864;
compounds which inhibit the signal transduction cascade, for example and preferably from the group of kinase inhibitors, in particular from the group of tyrosine kinase and/or serine/threonine kinase inhibitors;
compounds which inhibit soluble epoxide hydrolase (sEH), such as, for example, N,N′-dicyclohexylurea, 12-(3-adamantan-1-yl-ureido)dodecanoic acid or 1-adamantan-1-yl-3-{5-[2-(2-ethoxyethoxy)ethoxy]pentyl}urea;
compounds which influence the energy metabolism of the heart, such as by way of example and preferably etomoxir, dichloroacetate, ranolazine or trimetazidine;
agonists of VPAC receptors, such as by way of example and preferably the vasocactive intestinal polypeptide (VIP);
agents having an antithrombotic effect, for example and preferably from the group of platelet aggregation inhibitors, of anticoagulants or of profibrinolytic substances;
active ingredients which lower blood pressure, for example and preferably from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors and diurectics; and/or
active ingredients which alter lipid metabolism, for example and preferably from the group of thyroid receptor agonists, cholesterol synthesis inhibitors such as by way of example and preferably HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.

In a preferred embodiment of the invention, the compounds of the invention are employed in combination with a kinase inhibitor such as by way of example and preferably bortezomib, canertinib, erlotinib, gefitinib, imatinib, lapatinib, lestaurtinib, lonafarnib, pegaptinib, pelitinib, semaxanib, sorafenib, sunitinib, tandutinib, tipifarnib, vatalanib, fasudil, lonidamine, leflunomide, or Y-27632.

Agents having an antithrombotic effect preferably mean compounds from the group of platelet aggregation inhibitors, of anticoagulants or of profibrinolytic substances.

In a preferred embodiment of the invention, the compounds of the invention are administered in combination with a platelet aggregation inhibitor such as by way of example and preferably aspirin, clopidogrel, ticlopidine or dipyridamole.

In a preferred embodiment of the invention, the compounds of the invention are administered in combination with a thrombin inhibitor such as by way of example and preferably ximelagatran, melagatran, bivalirudin or clexane.

In a preferred embodiment of the invention, the compounds of the invention are administered in combination with a GPIIb/IIIa antagonist such as by way of example and preferably tirofiban or abciximab.

In a preferred embodiment of the invention, the compounds of the invention are administered in combination with a factor Xa inhibitor such as by way of example and preferably rivaroxaban, DU-176b, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or a low molecular weight (LMW) heparin derivative.

In a preferred embodiment of the invention, the compounds of the invention are administered in combination with a vitamin K antagonist such as by way of example and preferably coumarin.

Agents which lower blood pressure preferably mean compounds from the group of calcium antagonists, angiotensin All antagonists, ACE inhibitors, endothelin antagonists, renin inhibitors, alpha-receptor blockers, beta-receptor blockers, mineralocorticoid receptor antagonists, Rho kinase inhibitors, and diuretics.

In a preferred embodiment of the invention, the compounds of the invention are administered in combination with a calcium antagonist such as by way of example and preferably nifedipine, amlodipine, verapamil or diltiazem.

In a preferred embodiment of the invention, the compounds of the invention are administered in combination with an alpha-1 receptor blocker such as by way of example and preferably prazosin.

In a preferred embodiment of the invention, the compounds of the invention are administered in combination with a beta-receptor blocker such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds of the invention are administered in combination with an angiotensin All antagonist such as by way of example and preferably losartan, candesartan, valsartan, telmisartan or embusartan.

In a preferred embodiment of the invention, the compounds of the invention are administered in combination with an ACE inhibitor such as by way of example and preferably enalapril, captopril, lisinopril, ramipril, delapril, fosinopril, quinopril, perindopril or trandopril.

In a preferred embodiment of the invention, the compounds of the invention are administered in combination with an endothelin antagonist such as by way of example and preferably bosentan, darusentan, ambrisentan or sitaxsentan.

In a preferred embodiment of the invention, the compounds of the invention are administered in combination with a renin inhibitor such as by way of example and preferably aliskiren, SPP-600 or SPP-800.

In a preferred embodiment of the invention, the compounds of the invention are administered in combination with a mineralocorticoid receptor antagonist such as by way of example and preferably spironolactone or eplerenone.

In a preferred embodiment of the invention, the compounds of the invention are administered in combination with a Rho kinase inhibitor such as by way of example and preferably fasudil, Y-27632, SLx-2119, BF-66851, BF-66852, BF-66853, KI-23095, SB-772077, GSK-269962A or BA-1049.

In a preferred embodiment of the invention, the compounds of the invention are administered in combination with a diuretic such as by way of example and preferably furosemide.

Agents which alter lipid metabolism preferably mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the compounds of the invention are administered in combination with a CETP inhibitor such as by way of example and preferably torcetrapib (CP-529 414), JJT-705 or CETP vaccine (Avant).

In a preferred embodiment of the invention, the compounds of the invention are administered in combination with a thyroid receptor agonist such as by way of example and preferably D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins such as by way of example and preferably lovastatin, simvastatin, pravastatin, fluvastatin, atorvastatin, rosuvastatin, cerivastatin or pitavastatin.

In a preferred embodiment of the invention, the compounds of the invention are administered in combination with a squalene synthesis inhibitor such as by way of example and preferably BMS-188494 or TAK-475.

In a preferred embodiment of the invention, the compounds of the invention are administered in combination with an ACAT inhibitor such as by way of example and preferably avasimibe, melinamide, pactimibe, eflucimibe or SMP-797.

In a preferred embodiment of the invention, the compounds of the invention are administered in combination with an MTP inhibitor such as by way of example and preferably implitapide, BMS-201038, R-103757 or JTT-130.

In a preferred embodiment of the invention, the compounds of the invention are administered in combination with a PPAR-gamma agonist such as by way of example and preferably pioglitazone or rosiglitazone.

In a preferred embodiment of the invention, the compounds of the invention are administered in combination with a PPAR-delta agonist such as by way of example and preferably GW-501516 or BAY 68-5042.

In a preferred embodiment of the invention, the compounds of the invention are administered in combination with a cholesterol absorption inhibitor such as by way of example and preferably ezetimibe, tiqueside or pamaqueside.

In a preferred embodiment of the invention, the compounds of the invention are administered in combination with a lipase inhibitor such as by way of example and preferably orlistat.

In a preferred embodiment of the invention, the compounds of the invention are administered in combination with a polymeric bile acid adsorbent such as by way of example and preferably cholestyramine, colestipol, colesolvam, CholestaGel or colestimide.

In a preferred embodiment of the invention, the compounds of the invention are administered in combination with a bile acid reabsorption inhibitor such as by way of example and preferably ASBT (=IBAT) inhibitors such as, for example, AZD-7806, S-8921, AK-105, BARI-1741, SC-435 or SC-635.

In a preferred embodiment of the invention, the compounds of the invention are administered in combination with a lipoprotein(a) antagonist such as by way of example and preferably gemcabene calcium (CI-1027) or nicotinic acid.

The present invention further relates to medicaments comprising at least one of the compounds according to the invention, usually in combination with one or more inert, non-toxic, pharmaceutically suitable excipients, and their use for the purposes mentioned above.

The compounds of the invention may have systemic and/or local effects. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds of the invention can be administered in administration forms suitable for these administration routes.

Suitable for oral administration are administration forms which function according to the prior art and deliver the compounds of the invention rapidly and/or in a modified manner, and which contain the compounds of the invention in crystalline and/or amorphized and/or dissolved form, such as, for example, tablets (uncoated and coated tablets, for example having coatings which are resistant to gastric juice or are insoluble or dissolve with a delay and control the release of the compound of the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other routes of administration are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears and eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

Oral or parenteral administration are preferred, especially oral and intravenous administration.

The compounds of the invention can be converted into the stated administration forms. This can take place in a manner known per se by mixing with inert, non-toxic, pharmaceutically suitable excipients. These excipients include inter alia carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorings (e.g. inorganic pigments such as, for example, iron oxides) and masking flavors and/or odors.

It has generally proved to be advantageous on parenteral administration to administer amounts of about 0.001 to 1 mg/kg, preferably about 0.01 to 0.5 mg/kg of body weight to achieve effective results. On oral administration, the dosage is about 0.01 to 100 mg/kg, preferably about 0.01 to 20 mg/kg, and very particularly preferably 0.1 to 10 mg/kg of body weight.

It may nevertheless be necessary where appropriate to deviate from the stated amounts, in particular as a function of body weight, administration route, individual response to the active ingredient, type of preparation and time or interval over which administration takes place. Thus, in some cases it may be sufficient to make do with less than the aforementioned minimum amount, whereas in other cases the upper limit mentioned must be exceeded. Where relatively large amounts are administered, it may be advisable to distribute these in a plurality of single doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data of liquid/liquid solutions are, unless indicated otherwise, based in each case on the volume.

### A. EXAMPLES

### Abbreviations

abs. absolute
Ac acetyl
aq. aqueous, aqueous solution
Boc tert-butoxycarbonyl
Bu butyl
c concentration
CAN cerium(IV) ammonium nitrate
TLC thin-layer chromatography
DCI direct chemical ionization (in MS)
DIBAH diisobutylaluminum hydride
DIEA diisopropylethylamine (“Hünig base”)

### DMAP 4-N,N-dimethylaminopyridine

DME 1,2-dimethoxyethane

### DMF N,N-dimethylformamide

DMSO dimethyl sulfoxide
ee enantiomeric excess
EI electron impact ionization (in MS)
eq equivalent(s)
ESI electrospray ionization (in MS)
Et ethyl
m.p. melting point
GC-MS gas chromatography-coupled mass spectrometry
sat. saturated
h hour(s)
HPLC high pressure liquid chromatography
cat. catalytic
conc. concentrated
LC-MS liquid chromatography-coupled mass spectrometry
Me methyl
min minute(s)
Ms methanesulfonyl (mesyl)
MS mass spectrometry

### NBS N-bromosuccinimide

NMR nuclear magnetic resonance spectrometry
p-TsOH para-toluenesulfonic acid
Pd/C palladium on activated carbon
Ph phenyl
rac. racemic
RP reversed phase (in HPLC)
RT room temperature
Rₜ retention time (in HPLC)
TFA trifluoroacetic acid
THF tetrahydrofuran

### LC-MS and GC-MS Methods:

### Method 1 (LC-MS):

MS instrument type: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 2 (LC-MS):

Instrument: Micromass Quattro LCZ with HPLC Agilent Serie 1100; column: Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 208-400 nm.

### Method 3 (LC-MS):

MS instrument type: Waters ZQ; HPLC instrument type: Waters Alliance 2795; column: Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 210 nm.

### Method 4 (LC-MS):

Instrument: Micromass Quattro LCZ with HPLC Agilent Serie 1100; column: Phenomenex Synergi 2.5μ MAX-RP 100A Mercury, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

### Method 5 (LC-MS):

MS instrument type: Micromass ZQ; HPLC instrument type: Waters Alliance 2795; column: Phenomenex Synergi 2.5μ MAX-RP 100A Mercury, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

### Method 6 (GC-MS):

Instrument: Micromass GCT, GC6890; column: Restek RTX-35, 15 m×200 μm×0.33 μm; constant helium flow: 0.88 ml/min; oven: 70° C.; inlet: 250° C.; gradient: 70° C., 30° C./min→310° C. (maintained for 3 min).

### Method 7 (LC-MS):

MS instrument type: Micromass ZQ; HPLC instrument type: Waters Alliance 2795; column: Merck Chromolith SpeedROD RP-18e 100×4.6 mm; mobile phase A: water+500 μl 50% strength formic acid/l, mobile phase B: acetonitrile+500 μl 50% strength formic acid/l; gradient: 0.0 min 10% B→7.0 min 95% B→9.0 min 95% B; oven: 35° C.; flow rate: 0.0 min 1.0 ml/min→7.0 min 2.0 ml/min→9.0 min 2.0 ml/min; UV detection: 210 nm.

### Method 8 (LC-MS):

Instrument: Micromass Plattform LCZ with HPLC Agilent Serie 1100; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 9 (LC-MS):

Instrument: Micromass Quattro LCZ with HPLC Agilent Serie 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 10 (LC-MS):

Instrument: Micromass QuattroPremier with Waters HPLC Acquity; column: Thermo Hypersil GOLD 1.9μ, 50 mm×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; oven: 50° C.; flow rate: 0.33 ml/min; UV detection: 210 nm.

### Starting Materials and Intermediates:

### Example 1A

### tert-Butyl (2E,6R)-6-hydroxyhept-2-enoate

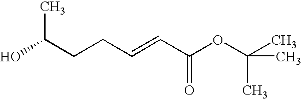

Solution A: 10.71 g (267.7 mmol) of 60% sodium hydride are suspended in 150 ml of abs. THF, and 43.3 ml (276.7 mmol) of tert-butyl P,P-dimethylphosphonoacetate are added dropwise with cooling. The mixture is stirred at RT, and after about 30 min a solution is formed.

187.4 ml (187.4 mmol) of a 1 M solution of DIBAH in THF are added dropwise to a solution, cooled to −78° C., of 17.87 g (178.5 mmol) of (R)-γ-valerolactone [(5R)-5-methyldihydrofuran-2(3H)-one] in 200 ml of abs. THF. The solution is stirred at −78° C. for 1 h, and solution A, prepared above, is then added. After the end of the addition, the mixture is slowly warmed to RT and stirred at RT overnight. The reaction mixture is added to 300 ml of ethyl acetate and extracted by stirring with 50 ml of concentrated potassium sodium tartrate solution. After phase separation, the aqueous phase is re-extracted with ethyl acetate. The organic phases are combined, washed with sat. sodium chloride solution, dried over magnesium sulfate and concentrated under reduced pressure. The residue is purified by chromatography on silica gel (mobile phase cyclohexane/ethyl acetate 5:1). This gives 32.2 g (90.1% of theory) of the target product which contains small amount of the cis-isomer.

MS (DCI): m/z=218 (M+NH₄)⁺

¹H-NMR (400 MHz, DMSO-d₆): δ=6.70 (dt, 1H), 5.73 (d, 1H), 4.44 (d, 1H), 3.58 (m, 1H), 2.28-2.13 (m, 2H), 1.47-1.40 (m, 2H), 1.45 (s, 9H), 1.04 (d, 3H).

### Example 2A

### tert-Butyl (−)-6-hydroxyheptanoate

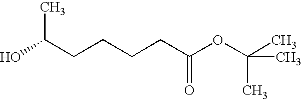

32.2 g (160.8 mmol) of tert-butyl (2E,6R)-6-hydroxyhept-2-enoate are dissolved in 200 ml of ethanol, and 1.7 g of 10% palladium on carbon are added. The mixture is stirred at RT under an atmosphere of hydrogen (atmospheric pressure) for 2 h and then filtered off through Celite. The filtrate is concentrated under reduced pressure. The residue gives, after chromatography on silica gel (mobile phase cyclohexane/ethyl acetate 10:1→6:1), 15.66 g of the target product (48.1% of theory).

MS (DCI): m/z=220 (M+NH₄)⁺

¹H-NMR (400 MHz, CDCl₃): δ=3.85-3.75 (m, 1H), 2.22 (t, 2H), 1.68-1.54 (m, 2H), 1.53-1.30 (m, 4H), 1.45 (s, 9H), 1.18 (d, 3H).

[α]_{D}²⁰=−21°, c=0.118, chloroform.

### Example 3A

### Methyl 6-[(6-phenylthieno[2,3-d]pyrimidin-4-yl)amino]hexanoate

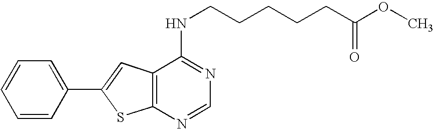

Under an atmosphere of argon, 500 mg (2.03 mmol) of 4-chloro-6-phenylthieno[2,3-d]pyrimidine are initially charged in 2 ml of DMF, and 1.8 ml (1309 mg, 10.13 mmol) of DIEA and 736 mg (4.05 mmol) of methyl 6-aminohexanoate hydrochloride are added. The reaction is stirred at a bath temperature of 120° C. for 1.5 h. For work-up, water is added to the cooled reaction mixture and the mixture is extracted three times with ethyl acetate. The combined extracts are washed with sat. sodium chloride solution and dried over magnesium sulfate, and the solvent is removed completely on a rotary evaporator. The residue is then chromatographed on silica gel (Biotage® cartridge) using a gradient of cyclohexane and ethyl acetate (4:1→2:1). This gives 590 mg of the target product (82% of theory).

LC-MS (method 1): Rₜ=2.54 min; m/z=356 (M+H)⁺.

### Example 4A

### Methyl 6-[(5-bromo-6-phenylthieno[2,3-d]pyrimidin-4-yl)amino]hexanoate

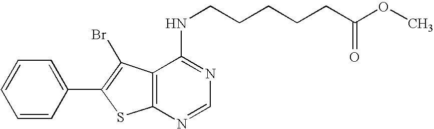

544 mg (1.53 mmol) of methyl 6-[(6-phenylthieno[2,3-d]pyrimidin-4-yl)amino]-hexanoate are suspended in 1.6 ml of acetonitrile, and 300 mg (1.68 mmol) of N-bromosuccinimide are then added. The reaction mixture is then stirred at RT for 1 h. Dichloromethane is added to the suspension, the mixture is washed successively with sat. sodium bicarbonate solution and sat. sodium chloride solution and the organic phase is dried over sodium sulfate and concentrated under reduced pressure. The residue is chromatographed on silica gel using a gradient of cyclohexane and ethyl acetate (5:1→4:1). This gives 563 mg of the target product (84% of theory).

LC-MS (method 1): Rₜ=3.01 min; m/z=434 (M+H)⁺

¹H-NMR (400 MHz, DMSO-d₆): δ=8.42 (s, 1H), 7.70-7.62 (m, 2H), 7.60-7.49 (m, 3H), 7.37 (t, 1H), 3.63-3.53 (m, 5H), 2.31 (t, 2H), 1.70-1.53 (m, 4H), 0.91-0.76 (m, 2H).

### Example 5A

### tert-Butyl (6R)-6-[(6-phenylthieno[2,3-d]pyrimidin-4-yl)oxy]heptanoate

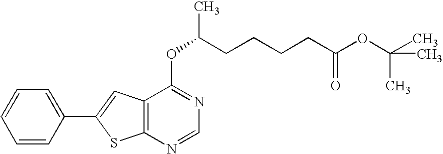

Under an atmosphere of argon, 500 mg (2.03 mmol) of 4-chloro-6-phenylthieno[2,3-d]pyrimidine and 615 mg (3.04 mmol) of tert-butyl (−)-6-hydroxyheptanoate are initially charged in 3.4 ml of THF and cooled to −20° C., and 2.6 ml (1670 mg, 2.64 mmol) of a 1 M solution of N′″-tert-butyl-N,N′,N″-tris[tris(dimethylamino)-phosphoranylidene]phosphorimidetriamide in hexane are added. The mixture is stirred at −20° C. for 30 min and then slowly warmed to 0° C. and stirred at this temperature for 1 h. For work-up, water is added and the reaction mixture is neutralized with 1 M hydrochloric acid and repeatedly extracted with dichloromethane. The combined extracts are dried over sodium sulfate and concentrated under reduced pressure. The residue is chromatographed on silica gel (Biotage® cartridge) using a mobile phase gradient of cyclohexane and ethyl acetate (30:1→20:1→10:1). This gives 249 mg of the target product (30% of theory).

LC-MS (method 2): Rₜ=5.01 min; m/z=413 (M+H)⁺

¹H-NMR (400 MHz, DMSO-d₆): δ=8.64 (s, 1H), 7.86 (d, 2H), 7.79 (s, 1H), 7.50 (t, 1H), 7.46-7.41 (m, 1H), 5.50 (m, 1H), 2.19 (t, 2H), 1.88-1.66 (m, 2H), 1.60-1.29 (m, 14H), 1.39 (d, 3H), 1.32 (s, 9H).

[α]_{D}²⁰=−71°, c=0.480, chloroform.

### Example 6A

### tert-Butyl (6R)-6-[(5-bromo-6-phenylthieno[2,3-d]pyrimidin-4-yl)oxy]heptanoate

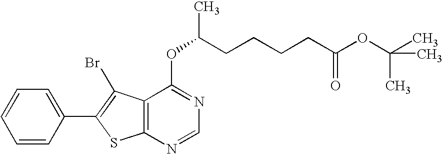

215 mg (0.52 mmol) of tert-butyl (6R)-6-[(6-phenylthieno[2,3-d]pyrimidin-4-yl)oxy]heptanoate are reacted with 102 mg (0.57 mmol) of N-bromosuccinimide in 0.6 ml of acetonitrile at RT for 2 h. Dichloromethane is then added to the suspension, the mixture is washed successively with sat. sodium bicarbonate solution and sat. sodium chloride solution and the organic phase is dried over sodium sulfate and concentrated under reduced pressure. The residue is chromatographed on silica gel 60 using a mobile phase of cyclohexane and ethyl acetate (10:1). This gives 214 mg of the target product (84% of theory).

LC-MS (method 3): Rₜ=5.04 min; m/z=491 (M+H)⁺

¹H-NMR (400 MHz, DMSO-d₆): δ=8.72 (s, 1H), 7.69 (dd, 2H), 7.61-7.49 (m, 3H), 5.51 (m, 1H), 2.19 (t, 2H), 1.90-1.65 (m, 2H), 1.61-1.28 (m, 14H), 1.39 (d, 3H), 1.32 (s, 9H).

### Example 7A

### 2-Amino-1-benzyl-4-(4-methoxyphenyl)-5-phenyl-1H-pyrrole-3-carbonitrile and 2-amino-1-benzyl-5-(4-methoxyphenyl)-4-phenyl-1H-pyrrole-3-carbonitrile (isomer mixture)

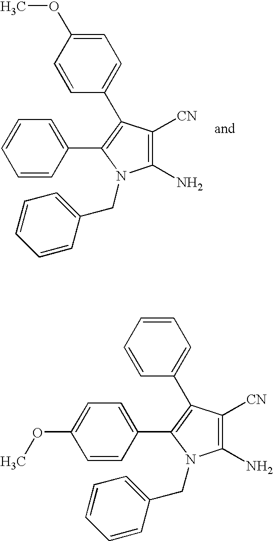

30.0 g (123.83 mmol) of 2-hydroxy-1-(4-methoxyphenyl)-2-phenylethanone are dissolved in 100 ml of toluene, and 13.5 ml (123.83 mmol) of benzylamine and 2.3 g (12.38 mmol) of p-toluenesulfonic acid monohydrate are added. On a water separator, the reaction mixture is stirred at boiling point for 2 h. 8.2 g (123.83 mmol) of malononitrile are then suspended in a little toluene and added dropwise continuously to the reaction mixture, which is boiling gently. The mixture is then stirred at boiling point for another 2 h. For work-up, the solvent is removed completely on a rotary evaporator, the residue is recrystallized from ethanol and the crystals are filtered off and dried under reduced pressure. This gives 15.3 g (33% of theory) of the target product as a mixture of regioisomers.

LC-MS (method 3): Rₜ=3.81 min; m/z=380 (M+H)⁺

¹H-NMR (400 MHz, DMSO-d₆): δ (for both isomers)=7.31-7.17 (m, 5H), 7.13 (d, 2H), 7.09-6.95 (m, 3H), 6.40-6.25 (m, 2H), 6.12 and 6.10 (2s, 2H), 4.96 and 4.93 (2s, 2H), 3.71 and 3.69 (2s, 3H).

### Example 8A

### 7-Benzyl-5-(4-methoxyphenyl)-6-phenyl-7H-pyrrolo[2,3-d]pyrimidine-4-amine and 7-benzyl-6-(4-methoxyphenyl)-5-phenyl-7H-pyrrolo[2,3-d]pyrimidine-4-amine (isomer mixture)

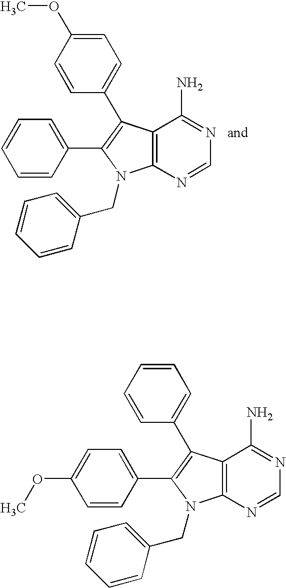

15.2 g (40.06 mmol) of the mixture of 2-amino-1-benzyl-4-(4-methoxyphenyl)-5-phenyl-1H-pyrrole-3-carbonitrile and 2-amino-1-benzyl-5-(4-methoxyphenyl)-4-phenyl-1H-pyrrole-3-carbonitrile are dissolved in a mixture of 60 ml of formamide, 20 ml of dimethylformamide and 8 ml of 99% strength formic acid and stirred under reflux at a bath temperature of about 190° C. for 10 h. For work-up, the reaction mixture is cooled to RT, the precipitated solid is filtered off and the filter residue is washed with 5% strength aqueous sodium hydroxide solution. The filter residue is recrystallized from ethanol and the crystals are once more filtered off (and then discarded). The filtrate from the recrystallization is evaporated to dryness on a rotary evaporator and the residue is dried under reduced pressure. This gives 12.6 g (45% of theory) of the target product as a mixture of regioisomers.

LC-MS (method 2): Rₜ=3.05 and 3.09 min; m/z in each case=407 (M+H)⁺

¹H-NMR (400 MHz, DMSO-d₆): δ (for both isomers)=8.17 and 8.19 (2s, 1H), 7.38-7.13 (m, 7H), 7.09 (d, 2H), 6.93-6.81 (m, 4H), 5.88 (br. s, 2H), 5.31 (s, 2H), 3.74 and 3.72 (2s, 3H).

### Example 9A

### 7-Benzyl-4-chloro-5-(4-methoxyphenyl)-6-phenyl-7H-pyrrolo[2,3-d]pyrimidine and 7-benzyl-4-chloro-6-(4-methoxyphenyl)-5-phenyl-7H-pyrrolo[2,3-d]pyrimidine (isomer mixture)

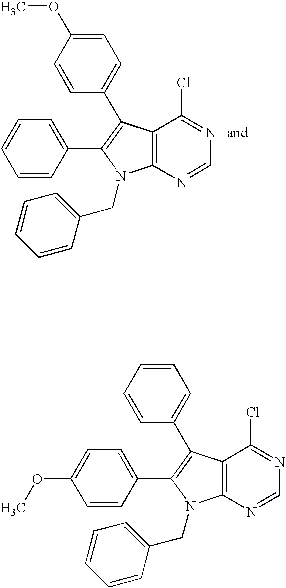

12.6 g (31.02 mmol) of the mixture of 7-benzyl-5-(4-methoxyphenyl)-6-phenyl-7H-pyrrolo[2,3-d]pyrimidine-4-amine and 7-benzyl-6-(4-methoxyphenyl)-5-phenyl-7H-pyrrolo[2,3-d]pyrimidine-4-amine are initially charged in 13.5 ml of chloroform, and 11.6 ml (1.7 g, 46.53 mmol) of a 4 M solution of hydrogen chloride in dioxane and 8.3 ml (7.3 g, 62.04 mmol) of isoamyl nitrite are added in succession. The reaction mixture is then stirred under reflux for 2.5 h. For work-up, the mixture is diluted with chloroform and then washed carefully with sat. sodium bicarbonate solution and sat. sodium chloride solution. The organic phase is dried over sodium sulfate and concentrated under reduced pressure. The crude product is chromatographed on silica gel (Biotage® cartridge) using a mobile phase of cyclohexane and ethyl acetate (20:1). This gives 2.4 g (18% of theory) of the target product as a mixture of regioisomers.

LC-MS (method 2): Rₜ=4.40 min; m/z=426 (M+H)⁺

¹H-NMR (400 MHz, DMSO-d₆): 6 (for both isomers)=8.71 and 8.69 (2s, 1H), 7.40-7.12 (m, 10H), 6.92-6.79 (m, 4H), 5.45 (s, 2H), 3.74 and 3.72 (2s, 3H).

### Example 10A

### tert-Butyl (6R)-6-{[7-benzyl-5-(4-methoxyphenyl)-6-phenyl-7H-pyrrolo[2,3-d]pyrimidin-4-yl]oxy}heptanoate

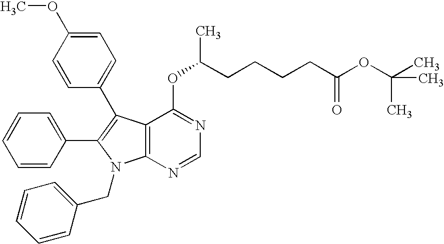

Under an atmosphere of argon, 800 mg (1.88 mmol) of the mixture of 7-benzyl-4-chloro-5-(4-methoxyphenyl)-6-phenyl-7H-pyrrolo[2,3-d]pyrimidine and 7-benzyl-4-chloro-6-(4-methoxyphenyl)-5-phenyl-7H-pyrrolo[2,3-d]pyrimidine and 570 mg (2.82 mmol) of tert-butyl (−)-6-hydroxyheptanoate are dissolved in 3.2 ml of THF, and 2.8 ml (1785 mg, 2.64 mmol) of a 1 M solution of N′″-tert-butyl-N,N′,N″-tris[tris(dimethylamino)phosphoranylidene]phosphorimidetriamide in hexane are added dropwise at 0° C. The reaction mixture is kept at 0° C. for 10 min and then warmed to RT and stirred at this temperature for another 1 h. For work-up, water is added and the mixture is neutralized with 1 M hydrochloric acid and extracted repeatedly with dichloromethane. The combined extracts are dried over sodium sulfate and concentrated under reduced pressure. The residue is pre-purified on silica gel (Biotage® cartridge) using a mobile phase of cyclohexane and ethyl acetate (10:1). The isomers are then separated by preparative RP-HPLC (column material: Phenomenex Gemini C18, 5 μm) over a running time of 30 min using a mobile phase of water and acetonitrile (20:80). This gives 234 mg (21% of theory) of the target product as a pure regioisomer.

LC-MS (method 2): Rₜ=5.12 min; m/z=592 (M+H)⁺

¹H-NMR (400 MHz, DMSO-d₆): δ=8.43 (s, 1H), 7.41-7.30 (m, 3H), 7.22-7.11 (m, 7H), 6.83 (m, 2H), 6.75 (d, 2H), 5.41-5.31 (m, 1H), 5.38 (s, 2H), 3.69 (s, 3H), 2.10 (t, 2H), 1.53 (m, 2H), 1.42 (m, 2H), 1.34 (s, 9H), 1.28-1.12 (m, 2H), 1.25 (d, 3H).

[α]_{D}²⁰=−48°, c=0.470, chloroform.

### Example 11A

### (6R)-6-{[7-Benzyl-5-(4-methoxyphenyl)-6-phenyl-7H-pyrrolo[2,3-d]pyrimidin-4-yl]oxy}heptanoic acid

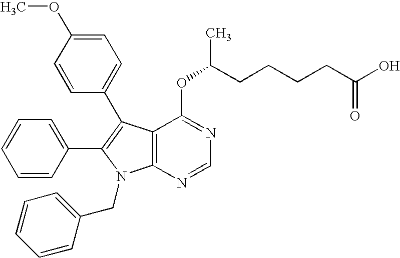

130 mg (0.22 mmol) of tert-butyl (6R)-6-{[7-benzyl-5-(4-methoxyphenyl)-6-phenyl-7H-pyrrolo[2,3-d]pyrimidin-4-yl]oxy}heptanoate are dissolved in 0.7 ml of dichloromethane, 0.2 ml of trifluoroacetic acid is added and the mixture is stirred at RT for 40 min. For work-up, the solvent is removed completely on a rotary evaporator and the residue is purified by preparative RP-HPLC. This gives 93 mg (79% of theory) of the target product.

LC-MS (method 4): Rₜ=2.68 min; m/z=536 (M+H)⁺

¹H-NMR (400 MHz, DMSO-d₆): δ=12.00 (br. s, 1H), 8.44 (s, 1H), 7.41-7.29 (m, 3H), 7.24-7.10 (m, 7H), 6.83 (dd, 2H), 6.75 (d, 2H), 5.41-5.29 (m, 1H), 5.38 (s, 2H), 3.69 (s, 3H), 2.11 (t, 2H), 1.53 (m, 2H), 1.42 (m, 2H), 1.32-1.13 (m, 2H), 1.25 (d, 3H).

[α]_{D}²⁰=−45°, c=0.490, chloroform.

### Example 12A

### 2-Phenyl-6,7-dihydro-1-benzofuran-4(5H)-one

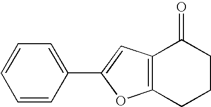

Under an atmosphere of argon, 107.6 g (196.20 mmol) of ammonium cerium(IV) nitrate and 37.5 g (445.91 mmol) of sodium bicarbonate are initially charged in 130 ml of acetonitrile. The suspension is cooled to 0° C. A solution of 10.0 g (89.18 mmol) of 1,3-cyclohexanedione and 27.3 g (267.55 mmol) of phenylacetylene in 20 ml of acetonitrile is added dropwise, and the mixture is stirred at RT for 3 h. For work-up, the solid is filtered off, the filter residue is washed with acetonitrile and the filtrate is concentrated under reduced pressure. The residue is taken up in ethyl acetate and washed successively with water, sat. sodium bicarbonate solution and sat. sodium chloride solution, and the organic phase is dried over magnesium sulfate. The solvent is removed completely on a rotary evaporator and the resulting brown oil is chromatographed on silica gel using a gradient of cyclohexane and ethyl acetate (20:1→10:1→8:1→6:1). This gives 6.9 g (36% of theory) of the target product.

LC-MS (method 2): Rₜ=3.43 min; m/z=213 (M+H)⁺

¹H-NMR (400 MHz, DMSO-d₆): δ=7.74 (d, 2H), 7.44 (t, 2H), 7.34 (t, 1H), 7.18 (s, 1H), 2.97 (t, 2H), 2.45 (t, 2H), 2.12 (m, 2H).

### Example 13A

### 3-Bromo-2-phenyl-6,7-dihydro-1-benzofuran-4(5H)-one

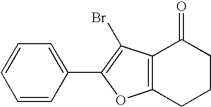

Under an atmosphere of argon, 6.9 g (32.42 mmol) of 2-phenyl-6,7-dihydro-1-benzofuran-4(5H)-one are suspended in 34.4 ml of acetonitrile, 6.9 g (38.90 mmol) of N-bromosuccinimide are added and the mixture is stirred at RT for 20 min. The mixture is then concentrated under reduced pressure, the residue is taken up in dichloromethane, the insoluble residue is filtered off and the filter residue is washed with dichloromethane. The combined filtrates are concentrated under reduced pressure and the residue is chromatographed on silica gel (Biotage® cartridge) using a mobile phase of dichloromethane and cyclohexane (30:1). This gives 7.6 g (80% of theory) of the target product.

LC-MS (method 3): Rₜ=3.56 min; m/z=291 (M+H)⁺

¹H-NMR (400 MHz, DMSO-d₆): δ=7.91 (d, 2H), 7.53 (t, 2H), 7.43 (t, 1H), 2.99 (t, 2H), 2.47 (t, 2H), 2.12 (m, 2H).

### Example 14A

### 3-(4-Methoxyphenyl)-2-phenyl-6,7-dihydro-1-benzofuran-4(5H)-one

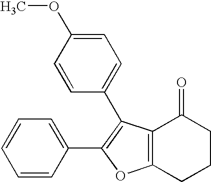

Under an atmosphere of argon, 6.8 g (23.18 mmol) of 3-bromo-2-phenyl-6,7-dihydro-1-benzofuran-4(5H)-one are dissolved in 22.5 ml of DMF, and 0.8 g (1.16 mmol) of bis(triphenylphosphine)palladium(II) chloride, 23.2 ml (4.9 g, 46.36 mmol) of 2 M aqueous sodium carbonate solution and 3.9 g (25.5 mmol) of 4-methoxyphenylboronic acid are added. The reaction mixture is stirred at 80° C. for 5 h. For work-up, dichloromethane is added to the cooled reaction mixture, the catalyst is filtered off through kieselguhr, the filter residue is washed with dichloromethane and the filtrate is concentrated under reduced pressure. The residue is chromatographed on silica gel using a gradient of cyclohexane and ethyl acetate (10:1→5:1). This gives 5.0 g (67% of theory) of the target product.

LC-MS (method 2): Rₜ=4.04 min; m/z=319 (M+H)⁺

¹H-NMR (400 MHz, DMSO-d₆): δ=7.35-7.18 (m, 5H), 7.21 (d, 2H), 6.93 (d, 2H), 3.79 (s, 3H), 2.99 (t, 2H), 2.43 (t, 2H), 2.13 (m, 2H).

### Example 15A

### 5-Bromo-3-(4-methoxyphenyl)-2-phenyl-6,7-dihydro-1-benzofuran-4(5H)-one

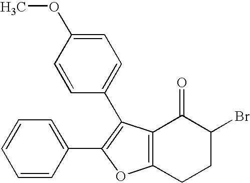

Under an atmosphere of argon, 500 mg (1.57 mmol) of 3-(4-methoxyphenyl)-2-phenyl-6,7-dihydro-1-benzofuran-4(5H)-one are dissolved in 3 ml of THF, and 590 mg (1.57 mmol) of phenyltrimethylammonium tribromide are added. The reaction mixture is stirred at RT for 6 h. For work-up, the solution is concentrated under reduced pressure and the residue is purified by preparative RP-HPLC using a gradient of water and acetonitrile. This gives 550 mg (88% of theory) of the target product.

LC-MS (method 5): Rₜ=2.49 min; m/z=397 (M+H)⁺

¹H-NMR (400 MHz, DMSO-d₆): δ=7.39-7.26 (m, 5H), 7.21 (d, 2H), 6.96 (d, 2H), 4.80 (t, 1H), 3.80 (s, 3H), 3.09 (m, 2H), 2.75-2.63 (m, 1H), 2.58-2.40 (m, 1H).

### Example 16A

### 3-(4-Methoxyphenyl)-2-phenyl-1-benzofuran-4-ol

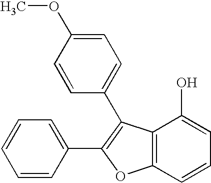

Under an atmosphere of argon, 1.0 g (2.52 mmol) of 5-bromo-3-(4-methoxyphenyl)-2-phenyl-6,7-dihydro-1-benzofuran-4(5H)-one are suspended in 20 ml of methanol, and 0.7 ml (0.5 g, 5.03 mmol) of triethylamine is added. After 4 h of stirring under reflux, another 0.7 ml (0.5 g, 5.03 mmol) of triethylamine is added and the mixture is stirred at boiling point overnight. The cooled reaction solution is poured into 1 N hydrochloric acid and extracted three times with ethyl acetate, and the combined extracts are washed with sat. sodium bicarbonate solution and sat. sodium chloride solution, dried over magnesium sulfate and concentrated under reduced pressure. The residue is chromatographed on silica gel (Biotage® cartridge) using a mobile phase of cyclohexane and ethyl acetate (10:1). The concentrated product fraction is triturated with a little methanol and the resulting crystals are filtered off and dried under reduced pressure. This gives 0.1 g (15% of theory) of the target product.

LC-MS (method 4): Rₜ=2.59 min; m/z=317 (M+H)⁺

¹H-NMR (400 MHz, DMSO-d₆): δ=9.60 (s, 1H), 7.43 (d, 2H), 7.38-7.25 (m, 5H), 7.15 (t, 1H), 7.08 (d, 1H), 6.98 (d, 2H), 6.61 (d, 1H), 3.81 (s, 3H).

### Example 17A

### tert-Butyl (+)-(6S)-6-bromoheptanoate

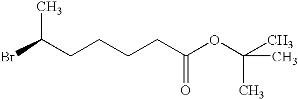

At 0° C., a solution of 2.3 g (5.4 mmol) of triphenylphosphine dibromide in 15 ml of abs. toluene is added dropwise to a solution of 1.0 g (4.9 mmol) of tert-butyl (−)-6-hydroxyheptanoate in 10 ml of abs. dichloromethane over a period of 1-2 h. After the end of the addition, the mixture is stirred at 0° C. for about 90 min. The resulting suspension is filtered through Celite and the filter residue is washed thoroughly with dichloromethane. The filtrate is washed with water, dried over sodium sulfate and concentrated under reduced pressure. The residue is chromatographed on silica gel (Biotage® cartridge, mobile phase cyclohexane/ethyl acetate 40:1). This gives 880.7 mg (67.2% of theory) of the target product.

GC-MS (method 6): Rₜ=4.48 min;

¹H-NMR (400 MHz, DMSO-d₆): δ=4.28 (m, 1H), 2.19 (t, 2H), 1.80-1.71 (m, 2H), 1.67 (d, 3H), 1.55-1.35 (m, 6H), 1.40 (5, 9H).

[α]_{D}²⁰=+30°, c=0.55, chloroform.

### Example 18A

### 4-Bromo-5-phenyl-2-furaldehyde

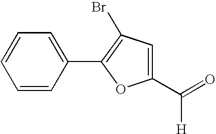

Under an atmosphere of argon, 17.1 g (95.83 mmol) of N-bromosuccinimide are added to 15.0 g (87.12 mmol) of 5-phenylfurfural suspended in 90 ml of acetonitrile. The reaction mixture is stirred at RT overnight. The mixture is then concentrated under reduced pressure, and the residue is chromatographed on silica gel using a mobile phase of cyclohexane and ethyl acetate (30:1). This gives 16.3 g (75% of theory) of the target product.

LC-MS (method 1): Rₜ=2.55 min; m/z=250 (M+H)⁺

¹H-NMR (400 MHz, DMSO-d₆): δ=9.61 (s, 1H), 8.02 (dd, 2H), 7.87 (s, 1H), 7.63-7.50 (m, 3H).

### Example 19A

### 4-(4-Methoxyphenyl)-5-phenyl-2-furaldehyde

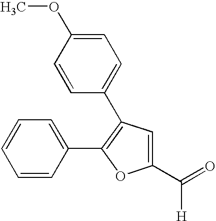

Under an atmosphere of argon, 1.1 g (1.62 mmol) of bis(triphenylphosphine)-palladium(II) chloride, 64.9 ml (13.76 g, 129.84 mmol) of 2 M aqueous sodium carbonate solution and 10.9 g (71.41 mmol) of 4-methoxyphenylboronic acid are added to 16.3 g (64.92 mmol) of 4-bromo-5-phenyl-2-furaldehyde dissolved in 90 ml of DMF. The reaction mixture is stirred at 80° C. for 2 h. For work-up, water is added to the cooled mixture and the mixture is extracted three times with dichloromethane. The combined extracts are dried over magnesium sulfate and concentrated under reduced pressure. The residue is chromatographed on silica gel using a mobile phase of cyclohexane and ethyl acetate (20:1). This gives 14.5 g (80% of theory) of the target product.

LC-MS (method 1): Rₜ=2.76 min; m/z=279 (M+H)⁺

¹H-NMR (400 MHz, DMSO-d₆): δ=9.64 (s, 1H), 7.74 (s, 1H), 7.55 (dd, 2H), 7.48-7.39 (m, 3H), 7.35 (d, 2H), 7.00 (d, 2H), 3.80 (s, 3H).

### Example 20A

### (2E)-3-[4-(4-Methoxyphenyl)-5-phenyl-2-furyl]acrylic acid

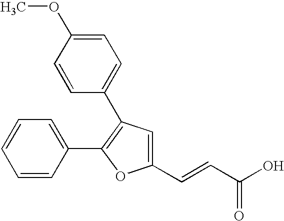

Under an atmosphere of argon, 5.0 g (17.97 mmol) of 4-(4-methoxyphenyl)-5-phenyl-2-furaldehyde and 1.9 g (17.97 mmol) of malonic acid are suspended in 9 ml of pyridine. After 1 h of stirring at 100° C., another 0.5 g (4.49 mmol) of malonic acid are added, and the mixture is stirred at 100° C. for a further 8 h. The cooled reaction solution is then poured into a mixture of ice-water and hydrochloric acid. Overnight, crystals precipitate from the acidified solution. The solid is filtered off, washed with water until neutral, dried under reduced pressure and chromatographed on silica gel using a gradient of dichloromethane and methanol (100:1→50:1→20:1→10:1). This gives 2.0 g (35% of theory) of the target product.

LC-MS (method 5): Rₜ=2.17 min; m/z=321 (M+H)⁺

¹H-NMR (400 MHz, DMSO-d₆): δ=12.43 (s, 1H), 7.55 (dd, 2H), 7.48-7.25 (m, 6H), 7.14 (s, 1H), 7.00 (d, 2H), 6.35 (d, 1H), 3.78 (s, 3H).

### Example 21A

### (2E)-3-[4-(4-Methoxyphenyl)-5-phenyl-2-furyl]acrylic acid azide

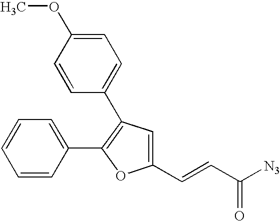

Under an atmosphere of argon, 8.2 g (25.66 mmol) of (2E)-3-[4-(4-methoxyphenyl)-5-phenyl-2-furyl]acrylic acid are suspended in 30 ml of acetone, 4.1 ml (29.77 mmol) of triethylamine and a further 30 ml of acetone are added and the mixture is cooled to −10° C. 3.1 ml (32.07 mmol) of ethyl chloroformate, dissolved in 6 ml of acetone, are added dropwise, and the mixture is stirred at 0° C. for 30 min. 2.5 g (38.49 mmol) of sodium azide, dissolved in 16 ml of water, are then added to the reaction mixture at 0° C., and the mixture is diluted with 10 ml of acetone. The mixture is warmed to RT and stirred for another 1 h. For work-up, the suspension is diluted with water, the precipitate is filtered off, the filter residue is washed with water until neutral and the solid is dried under reduced pressure. This gives 8.4 g (95% of theory) of the target product.

LC-MS (method 7): Rₜ=6.72 min; m/z=318 (M+H-N₂)⁺

¹H-NMR (400 MHz, DMSO-d₆): δ=7.65-7.55 (m, 3H), 7.44-7.27 (m, 6H), 7.01 (d, 2H), 6.45 (d, 1H), 3.79 (s, 3H).

### Example 22A

### 3-(4-Methoxyphenyl)-2-phenylfuro[3,2-c]pyridin-4(5H)-one

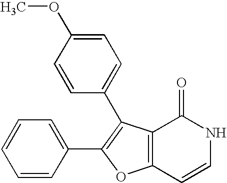

8.4 g (6.37 mmol) of (2E)-3-[4-(4-methoxyphenyl)-5-phenyl-2-furyl]acrylic acid azide are dissolved in 10.5 ml of toluene, 7.6 ml (5.9 g, 31.77 mmol) of tri-n-butylamine and 21.1 ml of Dowtherm A are added and the mixture is stirred under a strong stream of argon at a bath temperature of 230° C. for 2 h. The reaction mixture is then cooled in an ice-bath, resulting in the precipitation of crystals. The suspension is diluted with diethyl ether, the solid is filtered off and washed with diethyl ether and the product is dried under reduced pressure. This gives 5.1 g (65% of theory) of the target product.

LC-MS (method 5): Rₜ=1.90 min; m/z=318 (M+H)⁺

¹H-NMR (400 MHz, DMSO-d₆): δ=11.42 (d, 1H), 7.43 (d, 2H), 7.38-7.25 (m, 6H), 6.97 (d, 2H), 6.69 (d, 1H), 3.81 (s, 3H).

### Example 23A

### 4-Chloro-3-(4-methoxyphenyl)-2-phenylfuro[3,2-c]pyridine

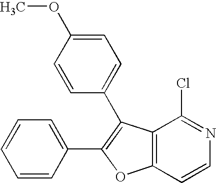

Under an atmosphere of argon, 5.1 g (15.91 mmol) of 3-(4-methoxyphenyl)-2-phenylfuro[3,2-c]pyridin-4(5H)-one are stirred in 30 ml (318 mmol) of phosphorus oxychloride at 130° C. for 10 h. The cooled reaction mixture is then diluted with ethyl acetate, ice-water is added carefully and the pH is adjusted to 6-7 using a mixture of sat. sodium carbonate solution and 10% strength aqueous sodium hydroxide solution. The organic phase is separated off, the aqueous phase is extracted twice with ethyl acetate and the combined extracts are dried over magnesium sulfate and concentrated under reduced pressure. The residue is triturated with methanol and the crystals are filtered off, washed with methanol and dried under reduced pressure. This gives 4.9 g (92% of theory) of the target product.

LC-MS (method 4): Rₜ=2.66 min; m/z=336 (M+H)⁺

¹H-NMR (400 MHz, DMSO-d₆): δ=8.33 (d, 1H), 7.85 (d, 1H), 7.52 (dd, 2H), 7.45-7.25 (m, 5H), 7.08 (d, 2H), 3.83 (s, 3H).

### Example 24A

### 3-{[3-(4-Methoxyphenyl)-2-phenylfuro[3,2-c]pyridin-4-yl]amino}-2,2-dimethylpropan-1-ol

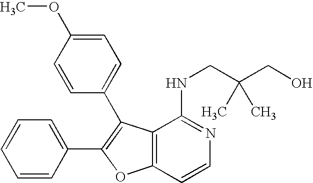

Under an atmosphere of argon, 200 mg (0.60 mmol) of 4-chloro-3-(4-methoxyphenyl)-2-phenylfuro[3,2-c]pyridine and 123 mg (1.19 mmol) of 3-amino-2,2-dimethyl-1-propanol are dissolved in 0.5 ml of DMF, 0.2 ml (1.19 mmol) of diisopropylethylamine are added and the mixture is stirred at 120° C. for 2 days. Water is added to the cooled reaction mixture, and the mixture is saturated with sodium chloride and extracted three times with ethyl acetate. The combined extracts are dried over magnesium sulfate and concentrated under reduced pressure. The residue is chromatographed on silica gel (Biotage® cartridge) using a gradient of cyclohexane and ethyl acetate (10:1→5:1→3:1→2:1). This gives 56 mg (23% of theory) of the target product.

LC-MS (method 1): Rₜ=1.86 min; m/z=403 (M+H)⁺

¹H-NMR (400 MHz, DMSO-d₆): δ=7.88 (d, 1H), 7.52-7.40 (m, 4H), 7.38-7.25 (m, 3H), 7.15 (d, 2H), 6.95 (d, 1H), 4.63 (t, 1H), 4.50 (t, 1H), 3.85 (s, 3H), 3.15 (d, 2H), 2.95 (d, 2H), 0.61 (s, 6H).

### Example 25A

### tert-Butyl 6-{[(benzyloxy)carbonyl]amino}hexanoate

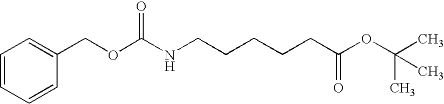

10.0 g (37.69 mmol) of 6-{[(benzyloxy)carbonyl]amino}hexanoic acid are suspended in a mixture of 21 ml of dichloromethane and 99 ml of cyclohexane. 9.8 g (45.23 mmol) of tert-butyl 2,2,2-trichloroacetimidate are added, 0.3 ml (566 mg, 3.77 mmol) of trifluoromethanesulfonic acid is added dropwise at 0-10° C. and the mixture is stirred at RT overnight. The solid formed is filtered off, the filter residue is washed with dichloromethane and the filtrate is concentrated under reduced pressure. The residue is chromatographed on silica gel 60 using a mobile phase of cyclohexane and ethyl acetate (5:1). This gives 4.9 g (40% of theory) of the target compound.

LC-MS (method 2): Rₜ=3.91 min; m/z=322 (M+H)⁺

¹H-NMR (400 MHz, DMSO-d₆): δ=7.39-7.25 (m, 5H), 7.22 (t, 1H), 4.99 (s, 2H), 2.97 (q, 2H), 2.15 (t, 2H), 1.48 (m, 2H), 1.43-1.33 (m, 2H), 1.38 (s, 9H), 1.25 (m, 2H).

### Example 26A

### tert-Butyl 6-aminohexanoate

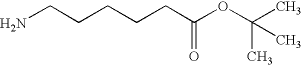

4.9 g (15.21 mmol) of tert-butyl 6-{[(benzyloxy)carbonyl]amino}hexanoate are initially charged in a mixture of 15 ml of ethanol and 1.5 ml of THF, 0.3 g (0.15 mmol) of 5% palladium on carbon are added and the mixture is hydrogenated at atmospheric pressure and RT for 4 h. The catalyst is filtered off through Celite, the filter residue is washed with ethanol/THF (10:1), the filtrate is concentrated under reduced pressure and the residue is dried under reduced pressure. This gives 2.8 g (97% of theory) of the target product.

LC-MS (method 8): Rₜ=2.41 min; m/z=188 (M+H)⁺

¹H-NMR (400 MHz, DMSO-d₆): δ=2.21-2.12 (m, 2H), 1.55-1.15 (m, 4H), 1.39 (s, 9H).

### Example 27A

### 1-Benzyl-4-(benzyloxy)pyridin-2(1H)-one

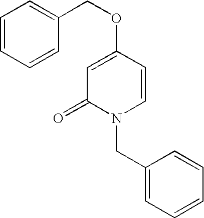

9.0 g (44.73 mmol) of 4-(benzyloxy)pyridin-2(1H)-one are, together with 8.94 g (223.63 mmol) of powdered sodium hydroxide and 6.074 g (17.89 mmol) of tetra-n-butylammonium hydrogensulfate, initially charged in 1.6 liter of toluene. 38.25 g (223.63 mmol) of benzyl bromide are added, and the mixture is heated at 80° C. with stirring for 2 hours. The mixture is then concentrated and the residue is dissolved in a mixture of 500 ml each of water and dichloromethane. The phases are separated and the aqueous phase is extracted once with 200 ml of dichloromethane. The combined dichloromethane phases are washed once with 100 ml of water and once with 100 ml of saturated sodium chloride solution. The dichloromethane phases are dried over magnesium sulfate and concentrated. The residue obtained is triturated with petroleum ether and the solid is filtered off with suction. The product is dried under high vacuum, giving 13.0 g (99.8% of theory) of the target compound.

LC-MS (method 3): Rₜ=3.21 min; m/z=292 (M+H)⁺

¹H-NMR (400 MHz, CDCl₃): δ=7.69 (d, 1H), 7.45-7.22 (m, 10H), 6.02 (dd, 1H), 5.94 (d, 1H), 5.08 (s, 2H), 5.02 (s, 2H).

### Example 28A

### 1-Benzyl-4-(benzyloxy)-3-iodopyridin-2(1H)-one

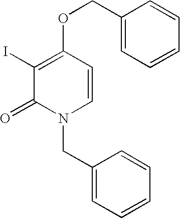

14.50 g (49.77 mmol) of 1-benzyl-4-(benzyloxy)pyridin-2(1H)-one are dissolved in 1 liter of acetonitrile. 20.16 g (89.58 mmol) of 1-iodopyrrolidine-2,5-dione are added, the reaction flask is wrapped in aluminum foil and the mixture is stirred at room temperature for 20 h. The mixture is then concentrated and the residue is purified by column chromatography on silica gel using a cyclohexane/ethyl acetate gradient (9:1→8:2→7:3→6:4→1:1). The product-containing fractions are concentrated and the residue is stirred at 50° C. with 250 ml of a cyclohexane/ethyl acetate mixture. The mixture is then once more cooled to 0° C., and the solid formed is filtered off with suction. In this manner, 15.5 g (74.6% of theory) of the target compound are obtained.

LC-MS (method 9): Rₜ=2.47 min; m/z=418 (M+H)⁺

¹H-NMR (400 MHz, CDCl₃): δ=7.92 (d, 1H), 7.48-7.38 (m, 4H), 7.37-7.24 (m, 6H), 6.39 (d, 1H), 5.31 (s, 2H), 5.12 (s, 2H).

### Example 29A

### 7-Benzyl-3-(4-ethylphenyl)-2-phenylfuro[2,3-b]pyridin-4(7H)-one

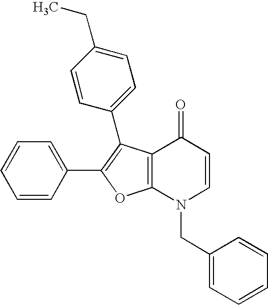

13.20 g (31.64 mmol) of 1-benzyl-4-(benzyloxy)-3-iodopyridin-2(1H)-one are, together with 26.4 ml of triethylamine, initially charged in 224 ml of acetonitrile. 1.11 g (1.58 mmol) of bis(triphenylphosphine)palladium(II) chloride, 301 mg (1.58 mmol) of copper(I) iodide and 4.20 g (41.13 mmol) of ethynylbenzene are added, and the mixture is, under argon and with stirring, heated at 60° C. for 22 h. The mixture is then allowed to cool to room temperature, 11.01 g (47.45 mmol) of 4-ethyliodobenzene are added and the mixture is once more, under argon and with stirring, heated at 60° C. for 24 h. The mixture is then concentrated and filtered through silica gel (mobile phase: cyclohexane/ethyl acetate 1:1, then dichloromethane/methanol 95:5). The product-containing fractions are combined and concentrated. The product obtained in this manner is once more purified by column chromatography on silica gel (mobile phase: dichloromethane/methanol 100:3). The product-containing fractions are once more combined and concentrated. The residue is dissolved in warm ethyl acetate, a little activated carbon is added, the mixture is briefly heated to the boil and the activated carbon is filtered off again. After cooling to room temperature, the precipitated crystals are filtered off with suction, and more crystals are obtained from the mother liquor. In this manner, a total of 6.00 g (44.1% of theory) of the target compound are obtained.

LC-MS (method 1): Rₜ=2.86 min; m/z=406 (M+H)⁺

¹H-NMR (400 MHz, CDCl₃): δ=7.85 (d, 1H), 7.49-7.23 (m, 11H), 7.22 (d, 2H), 6.05 (d, 1H), 5.45 (s, 2H), 2.69-2.61 (q, 2H), 1.26-1.21 (t, 3H).

### Example 30A

### 4-Chloro-(4-ethylphenyl)-2-phenylfuro[2,3-b]pyridine

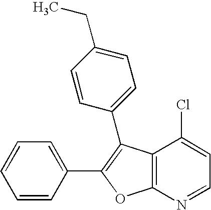

6.00 g (14.8 mmol) of 7-benzyl-3-(4-ethylphenyl)-2-phenylfuro[2,3-b]pyridin-4(7H)-one are initially charged in 150 ml of chloroform. At room temperature, 0.5 ml of DMF and then, slowly, 7.51 g (59.19 mmol) of oxalyl chloride are added, and the mixture is then stirred under reflux for 18 h. The mixture is then concentrated and the residue is purified by chromatography on silica gel (mobile phase: cyclohexane/ethyl acetate 85:15). This gives 3.22 g (65.2% of theory) of the target compound.

LC-MS (method 1): Rₜ=3.41 min; m/z=334 (M+H)⁺

¹H-NMR (400 MHz, CDCl₃): δ=8.20 (d, 1H), 7.62-7.58 (m, 2H), 7.35 (d, 2H), 7.31-7.25 (m, 5H), 7.18 (d, 1H), 2.80-2.72 (q, 2H), 1.35-1.30 (t, 3H).

### Example 31A

### 3-{[3-(4-Ethylphenyl)-2-phenylfuro[2,3-b]pyridin-4-yl]amino}-2,2-dimethylpropan-1-ol

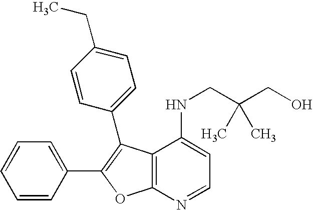

400 mg (1.2 mmol) of 4-chloro-(4-ethylphenyl)-2-phenylfuro[2,3-b]pyridine are, together with 618 mg (5.99 mmol) of 1-amino-2,2-dimethyl-3-propanol, dissolved in 4 ml of DMSO. The mixture is allowed to react in a microwave initially at 150° C. for 1 h and then at 200° C. for a further 2 h. The mixture is then diluted with 300 ml of water and extracted twice with in each case 200 ml of dichloromethane. The combined dichloromethane phases are dried over magnesium sulfate and concentrated. The product is purified by chromatography on silica gel (mobile phase: cyclohexane/ethyl acetate 1:1), giving 250 mg (52.1% of theory) of the target compound.

LC-MS (method 1): Rₜ=2.80 min; m/z=401 (M+H)⁺

¹H-NMR (400 MHz, CDCl₃): δ=7.97 (d, 1H), 7.56-7.52 (m, 2H), 7.42 (d, 2H), 7.35 (d, 2H), 7.26-7.21 (m, 3H), 6.28 (d, 1H), 4.42-4.38 (t, 1H), 3.16 (s, 2H), 2.93 (d, 2H), 2.78-2.71 (q, 2H), 1.32-1.28 (t, 3H), 0.68 (s, 6H).

### Example 32A

### 2-{[3-(4-Ethylphenyl)-2-phenylfuro[2,3-b]pyridin-4-yl]oxy}ethanol

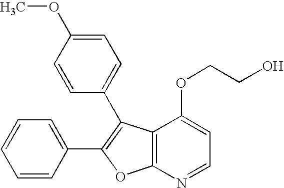

150 mg (0.45 mmol) of 4-chloro-3-(4-ethylphenyl)-2-phenylfuro[2,3-b]pyridine and 39 mg (0.63 mmol) of 1,2-ethanediol are dissolved in 1 ml of DMF, and 23 mg (0.58 mmol) of 60% sodium hydride are added at 0° C. The suspension is stirred at RT for 3 h and a further 45 min at 60° C. Separately, 39 mg (0.63 mmol) of 1,2-ethanediol and 23 mg (0.58 mmol) of 60% sodium hydride are then stirred at RT in DMF for 30 min, the suspension is filtered, the filtrate is, at RT, added dropwise to the above reaction solution, and the solution is then once more stirred at 60° C. overnight. This procedure is repeated with a further 39 mg (0.63 mmol) of 1,2-ethanediol and 23 mg (0.58 mmol) of 60% sodium hydride, and the reaction mixture is stirred at 60° C. for another 3 h. Then, after cooling, water is added, the mixture is extracted with ethyl acetate and the organic phase is washed with sat. sodium chloride solution, dried over magnesium sulfate and concentrated under reduced pressure. The residue is chromatographed on silica gel 60 using a mobile phase of dichloromethane and methanol (50:1). In this manner, 172 mg (27% of theory) of the target product are obtained.

LC-MS (method 1): Rₜ=2.79 min; m/z=360 (M+H)⁺

¹H-NMR (400 MHz, DMSO-d₆): δ=8.20 (d, 1H), 7.52 (dd, 2H), 7.45-7.29 (m, 5H), 7.25 (d, 2H), 6.98 (d, 1H), 4.64 (t, 1H), 4.11 (t, 2H), 3.53 (q, 2H), 2.68 (q, 2H), 1.24 (t, 3H).

### Example 33A

### 3-{[3-(4-Ethylphenyl)-2-phenylfuro[2,3-b]pyridin-4-yl]oxy}propan-1-ol

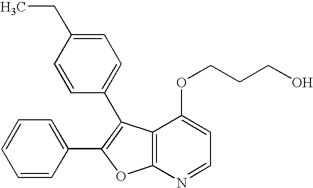

At 0° C., 15.6 mg (0.389 mmol, 60%) of sodium hydride are added to a solution of 100 mg (0.30 mmol) of 4-chloro-3-(4-ethylphenyl)-2-phenylfuro[2,3-b]pyridine and 30 μl (0.419 mmol) of 1,3-propanediol in 1.0 ml of DMF. The mixture is stirred initially at RT for 2 h and then at 60° C. overnight. After cooling, a further 1.4 eq. of 1,3-propanediol and 1.3 eq. 60% sodium hydride are added, and the reaction mixture is once more heated at 80° C. overnight. After cooling, the reaction mixture is diluted with water and extracted with ethyl acetate. The organic phase is dried over sodium sulfate and concentrated under reduced pressure and the residue is dried under high vacuum. Chromatography on silica gel (mobile phase: dichloromethane/methanol 50:1) gives 70 mg (60.4% of theory) of the target compound.

LC-MS (method 1): Rₜ=2.80 min; m/z=374 (M+H)⁺

¹H-NMR (400 MHz, DMSO-d₆): δ=8.19 (d, 1H), 7.53 (dd, 2H), 7.39-7.29 (m, 5H), 7.28 (d, 2H), 6.95 (d, 1H), 4.39 (t, 1H), 4.09 (t, 2H), 3.18 (q, 2H), 2.69 (q, 2H), 1.61 (m, 2H), 1.25 (t, 3H).

### Example 34A

### Methyl 3-nitrophenoxyacetate

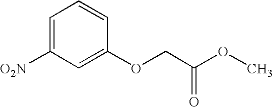

50 g (359.4 mmol) of 3-nitrophenol and 175.67 g (539 mmol) of cesium carbonate are initially charged in 1.0 liter of acetone, and 71.5 g (467.3 mmol) of methyl bromoacetate are added. The mixture is stirred at 50° C. for 1 h and, after cooling, poured into 7.5 liter of water. The suspension is stirred for 30 min and then filtered off with suction, and the filter residue is washed with water. The solid is dried in a drying cabinet at 50° C. and 100 mbar. This gives 64.3 g (84.7% of theory) of the target compound.

MS (DCI): m/z=229 (M+NH₄)⁺

¹H-NMR (300 MHz, CDCl₃): δ=7.90 (dd, 1H), 7.43 (t, 1H), 7.48 (t, 1H), 7.28 (dd, 1H), 4.75 (s, 2H), 3.86 (s, 3H).

### Example 35A

### Methyl 3-aminophenoxyacetate

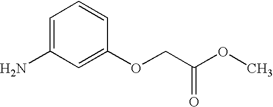

Under argon, 1.3 g of palladium on activated carbon (10%) are added to 13 g (61.6 mmol) of methyl 3-nitrophenoxyacetate in 150 ml of methanol. The mixture is stirred under an atmosphere of hydrogen (atmospheric pressure) at RT for 18 h. The catalyst is filtered off through kieselguhr and the filtrate is concentrated under reduced pressure. This gives, after drying under high vacuum, 10.7 g (95.9% of theory) of the target compound.

MS (DCI): m/z=199 (M+NH₄)⁺, 182 (M+H)⁺

¹H-NMR (400 MHz, CDCl₃): δ=7.10-7.02 (m, 1H), 6.35-6.23 (m, 2H), 4.58 (s, 2H), 3.79 (s, 3H), 3.65 (br. s, 2H).

### Working Examples

### General Procedure A

### Palladium-Catalyzed Arylation of Heteroaryl Bromides

10% by volume of methanol, the appropriate arylboronic acid (1.2 to 1.8 eq.), potassium carbonate (1.5 to 2.0 eq.) and bis(triphenylphosphine)palladium(II) chloride (0.04 to 0.1 eq.) are added successively to a solution of the heteroaryl bromide in question in DMSO (about 0.1 to 0.5 mol/l). Under argon, the reaction mixture is stirred at 80-100° C. for 2 to 8 h. After cooling, the crude mixture is separated by preparative RP-HPLC and the target product is isolated.

### The Working Examples Below are Obtained According to General Procedure A:

| Example | Structure | Analytical data |
|---|---|---|
| 1 | 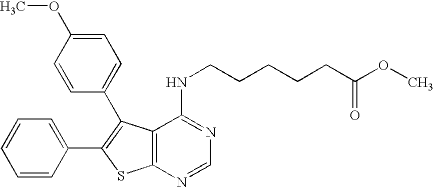 | LC-MS (method 2): Rₜ = 4.16 min; m/z = 462 (M + H)⁺. ¹H-NMR (400 MHz, DMSO-d₆): δ = 8.40 (s, 1H), 7.35 (d, 2H), 7.32- 7.22 (m, 5H), 7.07 (d, 2H), 4.84 (t, 1H), 3.82 (s, 3H), 3.58 (s, 3H), 3.27 (q, 2H), 2.23 (t, 2H), 1.41 (m, 2H), 1.32-1.20 (m, 2H), 1.07- 0.95 (m, 2H). |
| 2 | 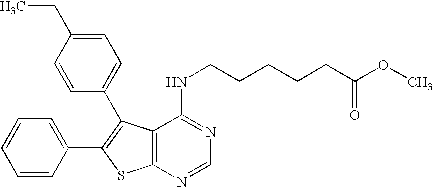 | LC-MS (method 2): Rₜ = 4.54 min; m/z = 460 (M + H)⁺. ¹H-NMR (400 MHz, DMSO-d₆): δ = 8.41 (s, 1H), 7.36 (s, 4H), 7.32- 7.22 (m, 5H), 4.68 (t, 1H), 3.57 (s, 3H), 3.23 (q, 2H), 2.69 (q, 2H), 2.23 (t, 2H), 1.40 (m, 2H), 1.28-1.14 (m, 5H), 1.08-0.95 (m, 2H). |
| 3 | 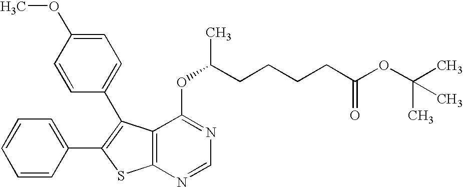 | LC-MS (method 3): Rₜ = 4.98 min; m/z = 519 (M + H)⁺. ¹H-NMR (400 MHz, DMSO-d₆): δ = 8.64 (s, 1H), 7.86-7.75 (m, 5H), 7.19 (d, 2H), 6.91 (d, 2H), 5.21 (m, 1H), 3.78 (s, 3H), 2.08 (t, 2H), 1.43-1.18 (m, 4H), 1.34 (s, 9H), 1.12 (d, 3H), 1.05-0.86 (m, 2H). [α]_{D}²⁰ = −60°, c = 0.475, chloroform. |
| 4 | 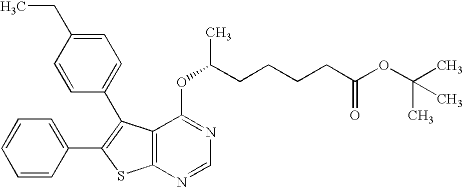 | LC-MS (method 3): Rₜ = 5.28 min; m/z = 517 (M + H)⁺. ¹H-NMR (400 MHz, DMSO-d₆): δ = 8.64 (s, 1H), 7.84-7.75 (m, 5H), 7.18 (m, 4H), 5.18 (m, 1H), 2.65 (q, 2H), 2.05 (t, 2H), 1.41-1.12 (m, 7H), 1.35 (s, 9H), 1.09 (d, 3H), 1.02- 0.90 (m, 2H). [α]_{D}²⁰ = −54°, c = 0.455, chloroform. |

### Example 5

### (6R)-6-{[5-(4-Methoxyphenyl)-6-phenyl-7H-pyrrolo[2,3-d]pyrimidin-4-yl]oxy}heptanoic acid

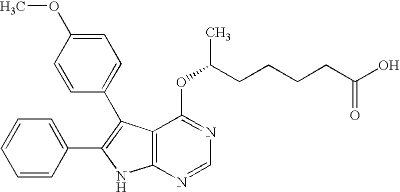

92 mg (0.17 mmol) of (6R)-6-{[7-benzyl-5-(4-methoxyphenyl)-6-phenyl-7H-pyrrolo[2,3-d]pyrimidin-4-yl]oxy}heptanoic acid are initially charged in a mixture of 30 ml of acetic acid and 3 ml of water, 10 mg of 10% palladium on carbon are then added and the mixture is stirred in an atmosphere of hydrogen at atmospheric pressure overnight. Two more times, in each case 10 mg of catalyst are then added and the mixture is hydrogenated at atmospheric pressure for a further 5 days. For work-up, the catalyst is filtered off, the filter residue is washed with acetic acid and the filtrate is concentrated under reduced pressure at 10-20° C. The residue is purified by preparative RP-HPLC (gradient of water and acetonitrile). This gives 5 mg (7.0% of theory) of the title compound.

LC-MS (method 1): Rₜ=2.59 min; m/z=446 (M+H)⁺

¹H-NMR (400 MHz, DMSO-d₆): δ=12.34 (s, 1H), 8.34 (s, 1H), 7.40 (d, 2H), 7.35-7.25 (m, 3H), 7.21 (d, 2H), 6.88 (d, 2H), 5.27 (m, 1H), 3.76 (s, 3H), 2.10 (t, 2H), 1.53-1.43 (m, 4H), 1.31-1.05 (m, 2H), 1.20 (d, 3H).

[α]_{D}²⁰=−24°, c=0.050, chloroform.

### General Procedure B

### Hydrolysis of Methyl or Ethyl Esters to the Corresponding Carboxylic Acids

At RT, 1.5 to 10 eq. of sodium hydroxide, as a 1 N aqueous solution, are added to a solution of the methyl or ethyl ester in THF or THF/methanol (1:1) (concentration about 0.05 to 0.5 mol/l). The mixture is stirred at RT for a period of 0.5-18 h and then neutralized or acidified slightly with 1 N hydrochloric acid. If a solid precipitates out, the product can be isolated by filtration, washing with water and drying under high vacuum. Alternatively, the target compound is isolated directly from the crude product, if appropriate after extractive work-up with dichloromethane, by preparative RP-HPLC (mobile phase: water/acetonitrile gradient).

### The Working Examples Below are Obtained According to General Procedure B:

| Example | Structure | Analytical data |
|---|---|---|
| 6 | 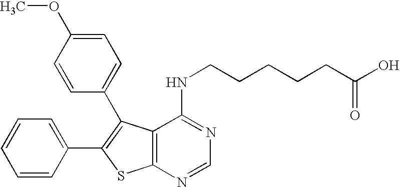 | LC-MS (method 1): Rₜ = 2.61 min; m/z = 448 (M + H)⁺. ¹H-NMR (400 MHz, DMSO-d₆): δ = 8.38 (s, 1H), 7.35 (d, 2H), 7.32- 7.22 (m, 5H), 7.08 (d, 2H), 4.84 (t, 1H), 3.82 (s, 3H), 3.24 (m, 2H), 1.92 (t, 2H), 1.38-1.18 (m, 4H), 1.04- 0.93 (m, 2H). |
| 7 | 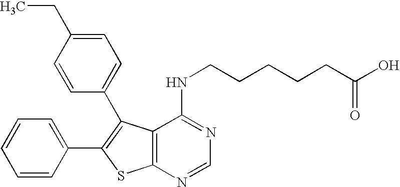 | LC-MS (method 1): Rₜ = 2.86 min; m/z = 448 (M + H)⁺. ¹H-NMR (500 MHz, CDCl₃): δ = 8.28 (s, 1H), 7.20-7.02 (m, 9H), 4.63 (m, 1H), 3.11 (m, 2H), 2.59 (m, 2H), 2.00 (m, 2H), 1.36 (m, 2H), 1.22-0.95 (m, 7H). |

### General Procedure C

### Cleavage of Tert-Butyl Esters to the Corresponding Carboxylic Acids

At from 0° C. to RT, TFA is added dropwise to a solution of the tert-butyl ester in dichloromethane (concentration from 0.05 to 1.0 mol/l; additionally one drop of water), until a dichloromethane/TFA ratio of about 2:1 to 1:1 is reached. The mixture is stirred at RT for 1-18 h and then concentrated under reduced pressure. The residue is purified by preparative RP-HPLC (mobile phase: acetonitrile/water gradient).

### The Working Examples Below are Obtained According to General Procedure C:

| Example | Structure | Analytical data |
|---|---|---|
| 8 | 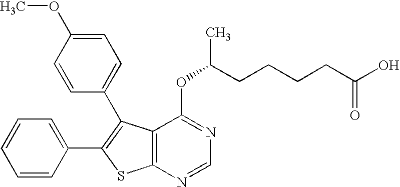 | LC-MS (method 3): Rₜ = 4.05 min; m/z = 463 (M + H)⁺. ¹H-NMR (400 MHz, DMSO-d₆): δ = 11.95 (s, 1H), 8.64 (s, 1H), 7.86- 7.75 (m, 5H), 7.19 (d, 2H), 6.90 (d, 2H), 5.20 (m, 1H), 3.78 (s, 3H), 2.08 (t, 2H), 1.43-1.18 (m, 4H), 1.12 (d, 3H), 1.07-0.88 (m, 2H). [α]_{D}²⁰ = −54°, c = 0.455, chloroform. |
| 9 | 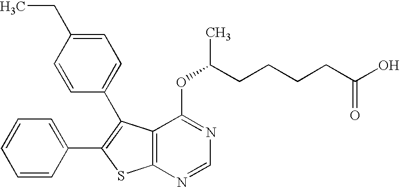 | LC-MS (method 3): Rₜ = 4.40 min; m/z = 461 (M + H)⁺. ¹H-NMR (400 MHz, DMSO-d₆): δ = 11.95 (br. s, 1H), 8.65 (s, 1H), 7.85- 7.75 (m, 5H), 7.18 (m, 4H), 5.18 (m, 1H), 2.65 (q, 2H), 2.07 (t, 2H), 1.40-1.12 (m, 4H), 1.20 (t, 3H), 1.09 (d, 3H), 1.03-0.89 (m, 2H). [α]_{D}²⁰ = −51°, c = 0.575, chloroform. |

### Example 10

### Ethyl 6-{[3-(4-methoxyphenyl)-2-phenyl-1-benzofuran-4-yl]oxy}hexanoate

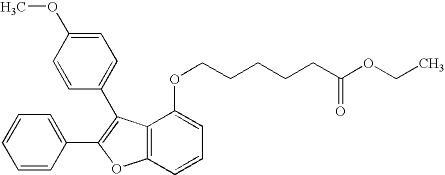

Under an atmosphere of argon, 100 mg (0.32 mmol) of 3-(4-methoxyphenyl)-2-phenyl-1-benzofuran-4-ol are dissolved in 0.6 ml of DMF, and 103 mg (0.32 mmol) of cesium carbonate, 10 mg (0.06 mmol) of potassium iodide and 88 mg (0.40 mmol) of ethyl 6-bromohexanoate are added. After 1 h of stirring at RT, another 51 mg (0.16 mmol) of cesium carbonate and 35 mg (0.16 mmol) of ethyl 6-bromohexanoate are added and the mixture is stirred at RT overnight. Water is added, the reaction mixture is saturated with sodium chloride and extracted with ethyl acetate, the combined extracts are dried over magnesium sulfate and the organic phase is concentrated under reduced pressure. The residue is purified by preparative RP-HPLC using a mobile phase of water and acetonitrile. 118 mg (81% of theory) of the target compound are isolated.

LC-MS (method 1): Rₜ=3.51 min; m/z=459 (M+H)⁺

¹H-NMR (400 MHz, DMSO-d₆): δ=7.49 (d, 2H), 7.38-7.21 (m, 7H), 6.99 (d, 2H), 6.71 (d, 1H), 4.05 (q, 2H), 3.85 (t, 2H), 3.70 (s, 3H), 2.15 (t, 2H), 1.48-1.31 (m, 4H), 1.18 (t, 3H), 0.94 (m, 2H).

### Example 11

### tert-Butyl (6R)-6-{[3-(4-methoxyphenyl)-2-phenyl-1-benzofuran-4-yl]oxy}heptanoate

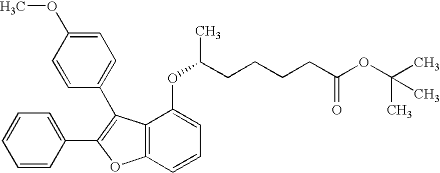

Under argon, 130 mg (0.41 mmol) of 3-(4-methoxyphenyl)-2-phenyl-1-benzofuran-4-ol of are initially charged in 0.5 ml of DMF, and 136.2 mg (0.51 mmol) of tert-butyl (+)-(6S)-6-bromoheptanoate, 133.9 mg (0.41 mmol) of cesium carbonate and 13 mg (0.08 mmol) of potassium iodide are added in succession. The mixture is stirred at RT overnight and then diluted with water. The mixture is saturated with sodium chloride and extracted three times with ethyl acetate. The combined organic phases are dried over magnesium sulfate and concentrated under reduced pressure. The crude product is purified by preparative RP-HPLC. 161 mg (78.3% of theory) of the target compound are isolated.

LC-MS (method 1): Rₜ=3.68 min; m/z=523 (M+Na)⁺

¹H-NMR (400 MHz, DMSO-d₆): δ=7.49 (d, 2H), 7.38-7.19 (m, 7H), 6.99 (d, 2H), 6.75 (d, 1H), 4.37 (m, 1H), 3.81 (s, 3H), 2.07 (t, 2H), 1.39 (s, 9H), 1.38-1.25 (m, 4H), 1.07 (d, 3H), 1.05-0.95 (m, 2H).

[α]_{D}²⁰=−63.5°, c=0.535, chloroform.

### Example 12

### 6-{[3-(4-Methoxyphenyl)-2-phenyl-1-benzofuran-4-yl]oxy}hexanoic acid

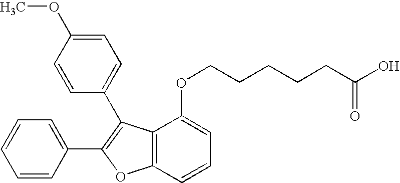

Under an atmosphere of argon, 90 mg (0.20 mmol) of ethyl 6-{[3-(4-methoxyphenyl)-2-phenyl-1-benzofuran-4-yl]oxy}hexanoate are suspended in 1 ml of ethanol, and 0.8 ml of 2.5 M aqueous sodium hydroxide solution is added. After 1 h of stirring at RT, another 0.8 ml of 2.5 M aqueous sodium hydroxide solution is added and the mixture is stirred at 40° C. for 1 h. For work-up, the cooled reaction solution is made slightly acidic using 1 M hydrochloric acid, and the resulting precipitate is filtered off. The precipitate is washed repeatedly with water and then triturated with a little methanol, filtered off and dried under reduced pressure. This gives 47 mg (56% of theory) of the title compound.

LC-MS (method 4): Rₜ=2.79 min; m/z=431 (M+H)⁺

¹H-NMR (400 MHz, DMSO-d₆): δ=11.98 (br. s, 1H), 7.48 (d, 2H), 7.38-7.20 (m, 7H), 6.99 (d, 2H), 6.71 (d, 1H), 3.87 (t, 2H), 3.71 (s, 3H), 2.09 (t, 2H), 1.48-1.28 (m, 4H), 0.94 (m, 2H).

### Example 13

### (6R)-6-{[3-(4-Methoxyphenyl)-2-phenyl-1-benzofuran-4-yl]oxy}heptanoic acid

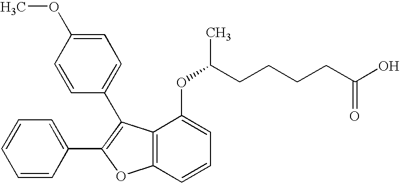

60 mg (0.12 mmol) of tert-butyl (6R)-6-{[3-(4-methoxyphenyl)-2-phenyl-1-benzofuran-4-yl]oxy}heptanoate are dissolved in 0.5 ml of dichloromethane. After addition of a drop of water, about 0.1 ml of trifluoroacetic acid is added and the mixture is stirred at RT for 30 min. Another 0.1 ml of TFA is added, and the mixture is then stirred for another 30 min. The reaction mixture is concentrated under reduced pressure and the residue is dried under high vacuum. The crude product is initially pre-purified by preparative RP-HPLC, and the product obtained is purified further by trituration with methanol and filtration. 7 mg (13.1% of theory) of the target compound are isolated.

LC-MS (method 10): Rₜ=3.68 min; m/z (ESIneg)=443 (M−H)⁻

¹H-NMR (400 MHz, DMSO-d₆): δ=11.96 (br. s, 1H), 7.50 (d, 2H), 7.38-7.18 (m, 7H), 6.99 (d, 2H), 6.73 (d, 1H), 4.36 (m, 1H), 3.82 (s, 3H), 2.09 (t, 2H), 1.38-1.22 (m, 4H), 1.05 (d, 3H), 1.05-0.95 (m, 2H).

### Example 14

### tert-Butyl (6R)-6-{[3-(4-methoxyphenyl)-2-phenylfuro[3,2-c]pyridin-4-yl]oxy}heptanoate

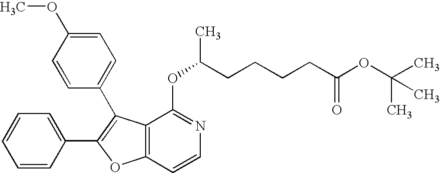

Under an atmosphere of argon, 100 mg (0.30 mmol) of 4-chloro-3-(4-methoxyphenyl)-2-phenylfuro[3,2-c]pyridine and 73 mg (0.36 mmol) of tert-butyl (−)-6-hydroxyheptanoate are dissolved in 1 ml of DMF. At 0° C., 0.4 ml (0.36 mmol) of a 1 M solution of N′″-tert-butyl-N,N′,N′″-tris[tris(dimethylamino)phosphoranylidene]-phosphorimidetriamide in hexane is then added. The reaction mixture is stirred at 0° C. for 1 h and then slowly warmed to RT and stirred at RT for a further 2 h. Water is added, and the mixture is saturated with sodium chloride and extracted three times with ethyl acetate. The combined extracts are dried over sodium sulfate and concentrated on a rotary evaporator. The crude product is purified by preparative RP-HPLC using a gradient of water and acetonitrile. This gives 21 mg of the target product (12.9% of theory).

LC-MS (method 4): Rₜ=3.44 min; m/z=502 (M+H)⁺

¹H-NMR (400 MHz, DMSO-d₆): δ=8.01 (d, 1H), 7.51 (d, 2H), 7.41-7.29 (m, 6H), 6.99 (d, 2H), 5.13 (m, 1H), 3.82 (s, 3H), 2.08 (t, 2H), 1.49-1.29 (m, 4H), 1.35 (s, 9H), 1.21-0.97 (m, 2H), 1.17 (d, 3H).

### Example 15

### tert-Butyl 6-{[3-(4-methoxyphenyl)-2-phenylfuro[3,2-c]pyridin-4-yl]amino}hexanoate

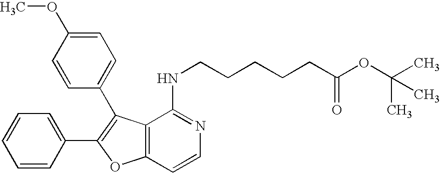

Under an atmosphere of argon, 100 mg (0.30 mmol) of 4-chloro-3-(4-methoxyphenyl)-2-phenylfuro[3,2-c]pyridine are initially charged in 1 ml of toluene, and 34 mg (0.36 mmol) of sodium tert-butoxide, 7 mg (0.01 mmol) of rac-2,2′-bis(diphenylphosphino)-1,1′-binaphthyl, 55 mg (0.06 mmol) of tris(dibenzylideneacetone)dipalladium and 89 mg (0.48 mmol) of tert-butyl 6-amino-hexanoate are successively added. The mixture is stirred under reflux for 1 h. The cooled reaction mixture is then diluted with dichloromethane, water is added, the catalyst and the salts are filtered off through Celite and the filter residue is washed with dichloromethane and concentrated under reduced pressure. The crude product is purified by preparative RP-HPLC (gradient of acetonitrile and water). This gives 96 mg (66% of theory) of the title compound.

LC-MS (method 5): Rₜ=1.82 min; m/z=487 (M+H)⁺

¹H-NMR (400 MHz, DMSO-d₆): δ=7.93 (d, 1H), 7.45 (d, 4H), 7.39-7.26 (m, 3H), 7.15 (m, 2H), 6.95 (d, 1H), 4.31 (t, 1H), 3.86 (s, 3H), 3.25 (q, 2H), 2.13 (t, 2H), 1.45-1.25 (m, 4H), 1.38 (s, 9H), 1.11-0.99 (m, 2H).

### Example 16

### tert-Butyl (3-{[3-(4-methoxyphenyl)-2-phenylfuro[3,2-c]pyridin-4-yl]amino}-2,2-dimethylpropoxy)acetate

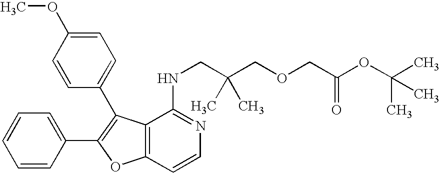

123 mg (1.54 mmol) of 50% strength aqueous sodium hydroxide solution and 0.2 ml of toluene are warmed to 40° C., and 5 mg (0.02 mmol) of tetra-N-butylammonium hydrogensulfate are added. A solution of 62 mg (0.15 mmol) of 3-{[3-(4-methoxyphenyl)-2-phenylfuro[3,2-c]pyridin-4-yl]amino}-2,2-dimethylpropan-1-ol in 0.2 ml of toluene and 46 μl (0.31 mmol) of tert-butyl bromoacetate are then added dropwise. The mixture is then stirred at 60° C. for 4 h. After cooling, the mixture is diluted with water and then extracted three times with ethyl acetate. The combined extracts are dried over magnesium sulfate and concentrated under reduced pressure. The crude product is purified by preparative RP-HPLC (gradient of water and acetonitrile). This gives 35 mg (56% of theory) of the title compound.

LC-MS (method 1): Rₜ=2.25 min; m/z=517 (M+H)⁺

¹H-NMR (400 MHz, DMSO-d₆): δ=7.90 (d, 1H), 7.50-7.40 (m, 4H), 7.37-7.26 (m, 3H), 7.15 (d, 2H), 6.94 (d, 1H), 4.49 (t, 1H), 3.85 (s, 3H), 3.75 (s, 2H), 3.25 (d, 2H), 2.98 (s, 2H), 1.40 (s, 9H), 0.69 (s, 6H).

### Example 17

### (6R)-6-{[3-(4-Methoxyphenyl)-2-phenylfuro[3,2-c]pyridin-4-yl]oxy}heptanoic acid

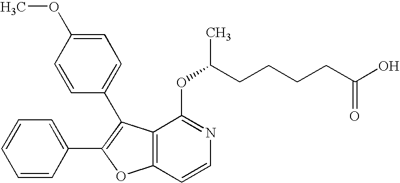

13 mg (0.03 mmol) of tert-butyl (6R)-6-{[3-(4-methoxyphenyl)-2-phenylfuro[3,2-c]pyridin-4-yl]oxy}heptanoate are dissolved in 1 ml of dichloromethane, 40 μl (59 mg, 0.52 mmol) of trifluoroacetic acid are added and the mixture is stirred at RT for 3 h. The reaction mixture is then diluted with dichloromethane and washed with water and sat. sodium chloride solution, the organic phase is dried over sodium sulfate and concentrated on a rotary evaporator and the residue is dried under reduced pressure. This gives 11 mg (93% of theory) of the target compound.

LC-MS (method 5): Rₜ=2.52 min; m/z=446 (M+H)⁺

¹H-NMR (400 MHz, DMSO-d₆): δ=11.95 (s, 1H), 8.01 (d, 1H), 7.52 (d, 2H), 7.41-7.29 (m, 6H), 7.01 (d, 2H), 5.14 (m, 1H), 3.82 (s, 3H), 2.10 (t, 2H), 1.50-1.30 (m, 4H), 1.21-0.97 (m, 2H), 1.17 (d, 3H).

[α]_{D}²⁰=−64°, c=0.420, chloroform.

### Example 18

### 6-{[3-(4-Methoxyphenyl)-2-phenylfuro[3,2-c]pyridin-4-yl]amino}hexanoic acid

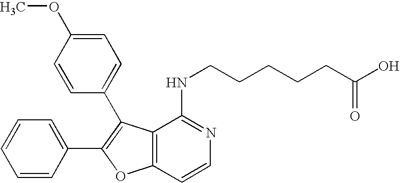

69 mg (0.14 mmol) of tert-butyl 6-{[3-(4-methoxyphenyl)-2-phenylfuro[3,2-c]pyridin-4-yl]amino}hexanoate are dissolved in 2 ml of dichloromethane, 0.2 ml of trifluoroacetic acid is added and the mixture is stirred at RT for 3 h. The reaction mixture is diluted with dichloromethane and washed with water and sat. sodium chloride solution, the organic phase is dried over sodium sulfate and concentrated under reduced pressure and the residue is chromatographed on silica gel (mobile phase: dichloromethane/methanol 10:1). This gives 48 mg (79% of theory) of the target compound.

LC-MS (method 1): Rₜ=1.81 min; m/z=431 (M+H)⁺

¹H-NMR (400 MHz, DMSO-d₆): δ=12.01 (br. s, 1H), 7.92 (d, 1H), 7.45 (d, 4H), 7.39-7.27 (m, 3H), 7.15 (m, 2H), 6.95 (d, 1H), 4.31 (t, 1H), 3.86 (s, 3H), 3.25 (q, 2H), 2.15 (t, 2H), 1.41 (m, 2H), 1.32 (m, 2H), 1.06 (m, 2H).

### Example 19

### (3-{[3-(4-Methoxyphenyl)-2-phenylfuro[3,2-c]pyridin-4-yl]amino}-2,2-dimethylpropoxy)acetic acid

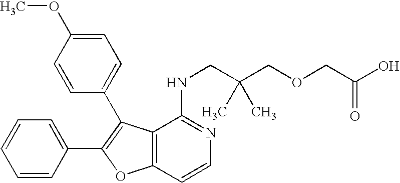

25 mg (0.05 mmol) of tert-butyl (3-{[3-(4-methoxyphenyl)-2-phenylfuro[3,2-c]pyridin-4-yl]amino}-2,2-dimethylpropoxy)acetate are stirred with 37 μl of trifluoroacetic acid and a drop of water at RT for 10 min. The trifluoroacetic acid and the water are then removed on a rotary evaporator, ethyl acetate is added to the residue and the mixture is washed once with sat. sodium bicarbonate solution. The aqueous phase is re-extracted twice with ethyl acetate. The combined organic phases are dried over magnesium sulfate and concentrated under reduced pressure. Drying of the residue under high vacuum gives 7 mg (32% of theory) of the title compound.

LC-MS (method 1): Rₜ=1.93 min; m/z=461 (M+H)⁺

¹H-NMR (400 MHz, DMSO-d₆): δ=12.54 (br. s, 1H), 7.90 (d, 1H), 7.50-7.40 (m, 4H), 7.36-7.26 (m, 3H), 7.16 (d, 2H), 6.94 (d, 1H), 4.47 (t, 1H), 3.85 (s, 3H), 3.79 (s, 2H), 3.25 (d, 2H), 2.99 (s, 2H), 0.69 (s, 6H).

### Example 20

### tert-Butyl 3-(3-{[3-(4-ethylphenyl)-2-phenylfuro[2,3-b]pyridin-4-yl]amino}-2,2-dimethylpropoxy)propanoate

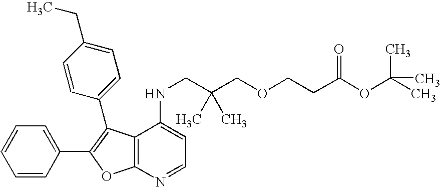

150 mg (0.375 mmol) of 3-{[3-(4-ethylphenyl)-2-phenylfuro[2,3-b]pyridin-4-yl]amino}-2,2-dimethylpropan-1-ol are, together with 240 mg (1.873 mmol) of tert-butyl acrylate and 25 mg (0.075 mmol) of tetra-N-butylammonium hydrogensulfate, initially charged in 3.75 ml of dichloromethane and cooled to 0° C. 0.75 ml of 50% strength aqueous sodium hydroxide solution is added, and the mixture is stirred vigorously at 0° C. for 20 min. The mixture is then allowed to warm to room temperature and stirred vigorously for another 3 h. The mixture is diluted with a little dichloromethane and water and acidified with 10% strength citric acid, and the phases are separated. The aqueous phase is re-extracted with dichloromethane. The combined organic phases are washed once with sat. sodium chloride solution, dried over magnesium sulfate and concentrated. The residue is purified on silica gel by preparative thick-layer chromatography (mobile phase: cyclohexane/ethyl acetate 7:3). The pure zone is scraped off and extracted with dichloromethane/methanol (95:5). The solvent is removed and the residue is dried under high vacuum, which gives 150 mg (75.8% of theory) of the target compound.

LC-MS (method 2): Rₜ=4.97 min; m/z=529 (M+H)⁺

¹H-NMR (400 MHz, CDCl₃): δ=7.98 (d, 1H), 7.56-7.51 (m, 2H), 7.42 (d, 2H), 7.34 (d, 2H), 7.26-7.19 (m, 3H), 6.29 (d, 1H), 4.26-4.22 (t, 1H), 3.52-3.48 (t, 2H), 2.92-2.87 (m, 4H), 2.78-2.71 (q, 2H), 2.41-2.36 (t, 2H), 1.41 (s, 9H), 1.31-1.26 (t, 3H), 0.64 (s, 6H).

### Example 21

### 3-(3-{[3-(4-Ethylphenyl)-2-phenylfuro[2,3-b]pyridin-4-yl]amino}-2,2-dimethylpropoxy)-propanoic acid

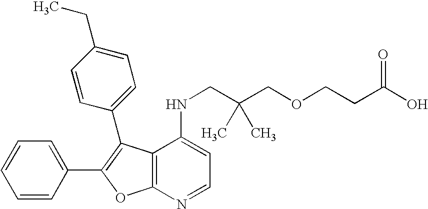

150 mg (0.284 mmol) of tert-butyl 3-(3-{[3-(4-ethylphenyl)-2-phenylfuro[2,3-b]pyridin-4-yl]amino}-2,2-dimethylpropoxy)propanoate are dissolved in 3 ml of dichloromethane. 0.75 ml of trifluoroacetic acid is added, and the mixture is stirred at room temperature for 2 h. The mixture is then evaporated to dryness, and the residue is purified on silica gel by preparative thick-layer chromatography (mobile phase dichloromethane/methanol 95:5). The pure zone is scraped off and extracted with dichloromethane/methanol (9:1). The solvent is removed and the residue is dried under high vacuum, which gives 85 mg (63.4% of theory) of the target compound.

LC-MS (method 1): Rₜ=2.87 min; m/z=473 (M+H)⁺

¹H-NMR (400 MHz, CDCl₃): δ=7.86 (d, 1H), 7.47-7.40 (m, 4H), 7.34 (d, 2H), 7.15 (m, 3H), 6.22 (d, 1H), 4.36-4.32 (t, 1H), 3.65-3.62 (t, 2H), 2.96-2.92 (m, 4H), 2.79-2.70 (q, 2H), 2.57-2.54 (t, 2H), 1.32-1.27 (t, 3H), 0.64 (s, 6H).

### Example 22

### tert-Butyl 6-{[3-(4-ethylphenyl)-2-phenylfuro[2,3-b]pyridin-4-yl]amino}hexanoate

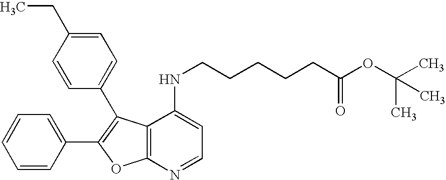

50 mg (0.15 mmol) of 4-chloro-3-(4-ethylphenyl)-2-phenylfuro[2,3-b]pyridine are initially charged in 1 ml of toluene, and 17 mg (0.18 mmol) of sodium tert-butoxide, 4 mg (0.01 mmol) of rac-2,2′-bis(diphenylphosphino)-1,1′-binaphthyl, 27 mg (0.03 mmol) of tris(dibenzylideneacetone)dipalladium and 45 mg (0.24 mmol) of tert-butyl 6-aminohexanoate are added in succession. The reaction mixture is stirred under reflux overnight. The cooled mixture is then diluted with dichloromethane, water is added, the catalyst is filtered off through Celite, the filtrate is washed with water and sat. sodium chloride solution and the organic phase is dried over sodium sulfate and concentrated under reduced pressure. The crude product is chromatographed on silica gel using a mobile phase of cyclohexane and ethyl acetate (2:1). This gives 44 mg (57% of theory) of the title compound.

LC-MS (method 1): Rₜ=3.52 min; m/z=485 (M+H)⁺

¹H-NMR (400 MHz, DMSO-d₆): δ=7.95 (d, 1H), 7.49-7.40 (m, 5H), 7.37-7.23 (m, 4H), 6.41 (d, 1H), 4.15 (t, 2H), 3.02 (q, 1H), 2.75 (q, 2H), 2.11 (t, 2H), 1.45-1.21 (m, 16H), 1.09-0.99 (m, 2H).

### Example 23

### tert-Butyl (3-{[3-(4-ethylphenyl)-2-phenylfuro[2,3-b]pyridin-4-yl]oxy}propoxy)acetate

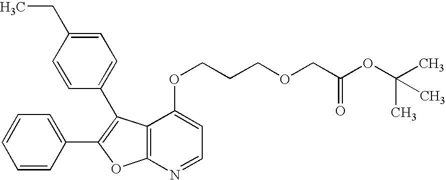

70 mg (0.19 mmol) of 3-{[3-(4-ethylphenyl)-2-phenylfuro[2,3-b]pyridin-4-yl]oxy}propan-1-ol and 30 μl (0.20 mmol) of tert-butyl bromoacetate are initially charged in 2 ml of DMF, 0.2 ml (126 mg, 0.20 mmol) of a 1 M solution of N′″-tert-butyl-N,N′,N″-tris[tris(dimethylamino)phosphoranylidene]phosphorimidetriamide in hexane is added at 0° C. and the mixture is stirred initially at 0° C. for 4 h and then at RT overnight. At 0° C., another 30 μl (0.20 mmol) of tert-butyl bromoacetate and 0.2 ml (126 mg, 0.20 mmol) of 1 M N′″-tert-butyl-N,N′,N″-tris[tris(dimethylamino)phosphoranylidene]phosphorimidetriamide solution in hexane are then added, and the mixture is stirred at RT for a further 12 h. Water is added, and the reaction mixture is neutralized with 1 M hydrochloric acid and extracted with dichloromethane. The extract is washed with sat. sodium chloride solution, dried over sodium sulfate and concentrated on a rotary evaporator. The crude product is pre-purified by chromatography on silica gel (gradient dichloromethane/methanol 100:1→50:1). Final fine purification by preparative RP-HPLC (gradient of water and acetonitrile) gives 19 mg (20% of theory) of the title compound.

LC-MS (method 5): Rₜ=2.88 min; m/z=488 (M+H)⁺

¹H-NMR (400 MHz, DMSO-d₆): δ=8.18 (d, 1H), 7.52 (dd, 2H), 7.39-7.29 (m, 5H), 7.27 (d, 2H), 6.93 (d, 1H), 4.55 (m, 1H), 3.79 (s, 2H), 3.12 (t, 2H), 2.69 (q, 2H), 1.70 (m, 2H), 1.39 (s, 9H), 1.23 (t, 3H).

### Example 24

### tert-Butyl (6R)-6-{[3-(4-ethylphenyl)-2-phenylfuro[2,3-b]pyridin-4-yl]oxy}heptanoate

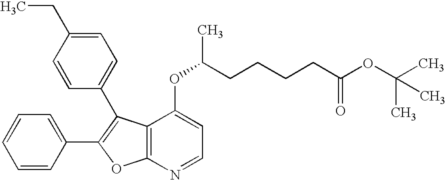

80 mg (0.24 mmol) of 4-chloro-3-(4-ethylphenyl)-2-phenylfuro[2,3-b]pyridine and 107 mg (0.53 mmol) of tert-butyl (−)-6-hydroxyheptanoate are dissolved in 1 ml of DMF. At 0° C., 0.5 ml (304 mg, 0.48 mmol) of a 1 M solution of N′″-tert-butyl-N,N′,N″-tris[tris(dimethylamino)phosphoranylidene]phosphorimidetriamide in hexane are then added and the reaction mixture is stirred at −5° C. to 0° C. for 2.5 h. Water is then added, and the mixture is neutralized with 1 M hydrochloric acid and extracted with dichloromethane. The organic phase is washed with sat. sodium chloride solution, dried over sodium sulfate and concentrated on a rotary evaporator. The crude product is chromatographed on silica gel (mobile phase: cyclohexane/ethyl acetate 3:1). This gives 27 mg (18% of theory) of the title compound.

LC-MS (method 5): Rₜ=3.19 min; m/z=500 (M+H)⁺

¹H-NMR (400 MHz, DMSO-d₆): δ=8.18 (d, 1H), 7.52 (dd, 2H), 7.41-7.30 (m, 5H), 7.28 (d, 2H), 6.93 (d, 1H), 4.57 (m, 1H), 2.74-2.64 (m, 2H), 2.07 (t, 2H), 1.45-1.20 (m, 3H), 1.34 (s, 9H), 1.23 (t, 3H), 1.13 (d, 3H), 1.03 (m, 2H).

### Example 25

### tert-Butyl 3-(2-{[3-(4-ethylphenyl)-2-phenylfuro[2,3-b]pyridin-4-yl]oxy}ethoxy)propanoate

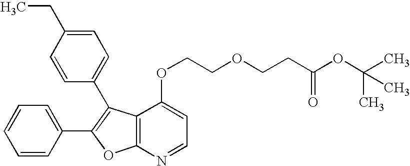

65 mg (0.18 mmol) of 2-{[3-(4-ethylphenyl)-2-phenylfuro[2,3-b]pyridin-4-yl]oxy}ethanol and 24 mg (0.19 mmol) of tert-butyl acrylate are dissolved in 2 ml of DMF, 0.2 ml (126 mg, 0.20 mmol) of a 1 M solution of N′″-tert-butyl-N,N′,N″-tris-[tris(dimethylamino)phosphoranylidene]phosphorimidetriamide in hexane is added at 0° C. and the mixture is stirred at 0° C. for 2.5 h. Water is then added to the reaction mixture, and the mixture is neutralized with 1 M hydrochloric acid and extracted with dichloromethane. The organic phase is washed with sat. sodium chloride solution, dried over sodium sulfate and concentrated on a rotary evaporator. The residue is dissolved in 2 ml of DMF, another 48 mg (0.38 mmol) of tert-butyl acrylate and 0.4 ml (252 mg, 0.40 mmol) of 1 M N′″-tert-butyl-N,N′,N″-tris[tris(dimethylamino)phosphoranylidene]phosphorimidetriamide solution in hexane are added at 0° C. and the mixture is stirred at RT overnight. The reactions are worked up as described above and the residue obtained is reacted at 0° C. with a further 232 mg (1.81 mmol) of tert-butyl acrylate and 8 mg (0.20 mmol) of 60% sodium hydride. The reaction mixture is then stirred at RT for a further night. After the extractive work-up described above, the dried residue is finally purified by preparative RP-HPLC (gradient of water and acetonitrile). In this manner, 22 mg (25% of theory) of the title compound are obtained.

LC-MS (method 4): Rₜ=3.05 min; m/z=488 (M+H)⁺

¹H-NMR (400 MHz, DMSO-d₆): δ=8.21 (d, 1H), 7.51 (dd, 2H), 7.41-7.30 (m, 5H), 7.27 (d, 2H), 6.95 (d, 1H), 4.15 (t, 2H), 3.51 (t, 2H), 3.38 (t, 2H), 2.68 (q, 2H), 2.31 (t, 2H), 1.35 (s, 9H), 1.24 (t, 3H).

### The Working Examples Below are Obtained According to General Procedure C:

| Example | Structure | Analytical data |
|---|---|---|
| 26 | 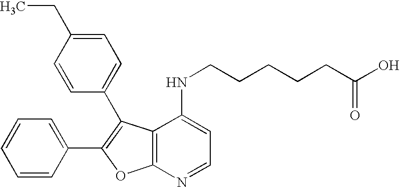 | LC-MS (method 4): Rₜ = 2.30 min; m/z = 429 (M + H)⁺. ¹H-NMR (400 MHz, DMSO-d₆): δ = 12.01 (br. s, 1H), 7.95 (d, 1H), 7.49- 7.39 (m, 6H), 7.38-7.25 (m, 3H), 6.41 (d, 1H), 4.15 (t, 1H), 3.02 (q, 2H), 2.73 (q, 2H), 2.14 (t, 2H), 1.39 (m, 2H), 1.33-1.22 (m, 5H), 1.05 (m, 2H). |
| 27 | 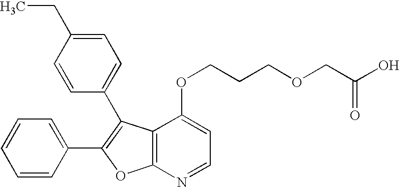 | LC-MS (method 1): Rₜ = 2.83 min; m/z = 432 (M + H)⁺. ¹H-NMR (400 MHz, DMSO-d₆): δ = 8.21 (d, 1H), 7.51 (dd, 2H), 7.41- 7.30 (m, 5H), 7.28 (d, 2H), 6.94 (d, 1H), 4.08 (t, 2H), 3.63 (m, 2H), 3.17 (t, 2H), 2.73-2.64 (m, 2H), 1.69 (m, 2H), 1.24 (t, 3H). |
| 28 | 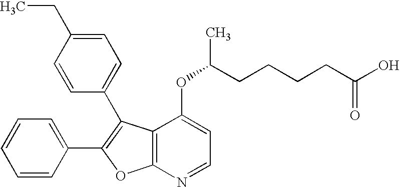 | LC-MS (method 4): Rₜ = 2.67 min; m/z = 444 (M + H)⁺. ¹H-NMR (400 MHz, DMSO-d₆): δ = 11.95 (br. s, 1H), 8.17 (d, 1H), 7.52 (dd, 2H), 7.40-7.31 (m, 5H), 7.27 (d, 2H), 6.93 (d, 1H), 4.55 (m, 1H), 2.69 (q, 2H), 2.09 (t, 2H), 1.45-1.19 (m, 4H), 1.25 (t, 3H), 1.12 (d, 3H), 1.05 (m, 2H). [α]_{D}²⁰ = −94°, c = 0.090, chloroform. |
| 29 | 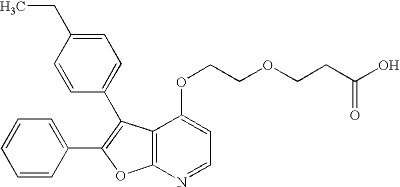 | LC-MS (method 1): Rₜ = 2.78 min; m/z = 432 (M + H)⁺. ¹H-NMR (400 MHz, CD₃OD): δ = 8.16 (d, 1H), 7.55 (m, 2H), 7.39 (d, 2H), 7.33- 7.24 (m, 5H), 6.91 (d, 1H), 4.20 (m, 2H), 3.59 (m, 2H), 3.45 (t, 2H), 2.74 (m, 2H), 2.40 (t, 2H), 1.37-1.25 (m, 3H). |

### Example 30

### Methyl (3-{[3-(4-methoxyphenyl)-2-phenylfuro[3,2-c]pyridin-4-yl]amino}phenoxy)acetate

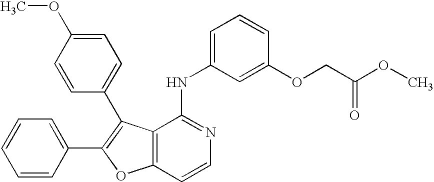

200 mg (0.596 mmol) of 4-chloro-3-(4-methoxyphenyl)-2-phenylfuro[3,2-c]pyridine, 129.5 mg (0.715 mmol) of methyl 3-aminophenoxyacetate and 135 μl (0.893 mmol) of triethylamine are mixed and heated to about 200° C. using a hot-air blower. Using the hot-air blower, the melt formed is heated even more and then briefly (for a few minutes) heated to the boil. After cooling, the target product is isolated directly by chromatography of the reaction mixture on silica gel (Biotage, gradient: cyclohexane/ethyl acetate 10:1→5:1). In this manner, 75 mg (26.2% of theory) of the title compound are obtained.

LC-MS (method 1): Rₜ=2.90 min; m/z=481 (M+H)⁺

¹H-NMR (400 MHz, DMSO-d₆): δ=8.12 (d, 1H), 7.59 (d, 2H), 7.53 (d, 2H), 7.42-7.34 (m, 3H), 7.29-7.24 (m, 4H), 7.14 (t, 1H), 6.64 (d, 1H), 6.55 (s, 1H), 5.98 (d, 1H), 4.72 (s, 2H), 3.92 (s, 3H), 3.71 (s, 3H).

### Example 31

### (3-{[3-(4-Methoxyphenyl)-2-phenylfuro[3,2-c]pyridin-4-yl]amino}phenoxy)acetic acid

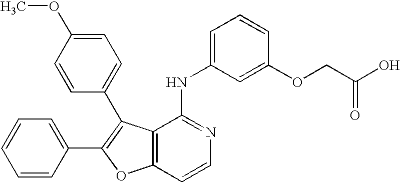

54.0 mg (0.112 mmol) of methyl (3-{[3-(4-methoxyphenyl)-2-phenylfuro[3,2-c]pyridin-4-yl]amino}phenoxy)acetate are initially charged in 0.5 ml of methanol, and 1.1 ml of 1 N aqueous sodium hydroxide solution are added at RT. After 30 min of stirring, 1 N hydrochloric acid is added and the reaction mixture is extracted three times with ethyl acetate. The combined organic phases are dried over magnesium sulfate and concentrated under reduced pressure. The residue is purified by preparative RP-HPLC (water/acetonitrile gradient). The product obtained in this manner is purified further by chromatography on silica gel (gradient: dichloromethane→dichloromethane/methanol 10:1). This gives 32 mg of the title compound (61% of theory).

LC-MS (method 10): Rₜ=1.25 min; m/z=467 (M+H)⁺

¹H-NMR (400 MHz, DMSO-d₆): δ=8.15 (d, 1H), 7.60 (d, 2H), 7.52 (d, 2H), 7.42-7.33 (m, 3H), 7.30-7.25 (m, 4H), 7.11 (t, 1H), 6.61 (dd, 1H), 6.53 (s, 1H), 6.45 (dd, 1H), 4.54 (s, 2H), 3.93 (s, 3H).

### B. ASSESSMENT OF PHARMACOLOGICAL EFFICACY

The pharmacological action of the compounds according to the invention can be demonstrated in the following assays:

### B-1. Studies of Binding to Prostacyclin Receptors (IP Receptors) of Human Thrombocyte Membranes

Thrombocyte membranes are obtained by centrifuging 50 ml of human blood (Buffy coats with CDP Stabilizer, from Maco Pharma, Langen) for 20 min at 160×g. Remove the supernatant (platelet-rich plasma, PRP) and then centrifuge again at 2000×g for 10 min at room temperature. Resuspend the sediment in 50 mM tris(hydroxymethyl)aminomethane, which has been adjusted to a pH of 7.4 with 1 N hydrochloric acid, and store at −20° C. overnight. On the next day, centrifuge the suspension at 80 000×g and 4° C. for 30 min. Discard the supernatant. Resuspend the sediment in 50 mM tris(hydroxymethyl)aminomethane/hydrochloric acid, 0.25 mM ethylene diamine tetraacetic acid (EDTA), pH 7.4, and then centrifuge once again at 80 000×g and 4° C. for 30 min. Take up the membrane sediment in binding buffer (50 mM tris(hydroxymethyl)-aminomethane/hydrochloric acid, 5 mM magnesium chloride, pH 7.4) and store at −70° C. until the binding test.

For the binding test, incubate 3 nM ³H-Iloprost (592 GBq/mmol, from AmershamBioscience) for 60 min with 300-1000 μg/ml of human thrombocyte membranes per charge (max. 0.2 ml) in the presence of the test substances at room temperature. After stopping, add cold binding buffer to the membranes and wash with 0.1% bovine serum albumin. After adding Ultima Gold Scintillator, quantify the radioactivity bound to the membranes using a scintillation counter. The nonspecific binding is defined as radioactivity in the presence of 1 μM Iloprost (from Cayman Chemical, Ann Arbor) and is as a rule <25% of the bound total radioactivity. The binding data (IC₅₀ values) are determined using the program GraphPad Prism Version 3.02.

### Representative Results for the Compounds According to the Invention are Shown in Table 1:

TABLE 1

| Example No. | IC₅₀ [nM] |
|---|---|
| 5 | 39 |
| 9 | 208 |
| 12 | 984 |
| 19 | 98 |
| 21 | 35 |
| 28 | 39 |
| 29 | 1703 |

### B-2. IP-Receptor Stimulation on Whole Cells

The IP-agonistic action of test substances is determined by means of the human erythroleukaemia cell line (HEL), which expresses the IP-receptor endogenously [Murray, R., FEBS Letters 1989, 1: 172-174]. For this, the suspension cells (4×10⁷ cells/ml) are incubated with the particular test substance for 5 minutes at 30° C. in buffer [10 mM HEPES (4-(2-hydroxyethyl)-1-piperazineethanesulfonic acid)/PBS (phosphate-buffered saline, from Oxoid, UK)], 1 mM calcium chloride, 1 mM magnesium chloride, 1 mM IBMX (3-isobutyl-1-methylxanthine), pH 7.4. Next, the reaction is stopped by addition of 4° C. cold ethanol and the charges are stored for a further 30 minutes at 4° C. Then the samples are centrifuged at 10 000×g and 4° C. The resultant supernatant is discarded and the sediment is used for determination of the concentration of cyclic adenosine monophosphate (cAMP) in a commercially available cAMP-radioimmunoassay (from IBL, Hamburg). In this test, IP agonists lead to an increase in cAMP concentration, but IP antagonists have no effect. The effective concentration (EC₅₀ value) is determined using the program GraphPad Prism Version 3.02.

### B-3. Inhibition of Thrombocyte Aggregation In Vitro

Inhibition of thrombocyte aggregation is determined using blood from healthy test subjects of both sexes. Mix 9 parts blood with one part 3.8% sodium citrate solution as coagulant. Centrifuge the blood at 900 rev/min for 20 min. Adjust the pH value of the platelet-rich plasma obtained to pH 6.5 with ACD solution (sodium citrate/citric acid/glucose). Then remove the thrombocytes by centrifugation, take up in buffer and centrifuge again. Take up the thrombocyte deposit in buffer and additionally resuspend with 2 mmol/l calcium chloride.

For the measurements of aggregation, incubate aliquots of the thrombocyte suspension with the test substance for 10 min at 37° C. Next, aggregation is induced by adding ADP and is determined by the turbidometric method according to Born in the aggregometer at 37° C. [Born G. V. R., J. Physiol. (London) 168, 178-179 (1963)].

### B-4. Measurement of Blood Pressure of Anaesthetized Rats

Anaesthetize male Wistar rats with a body weight of 300-350 g with thiopental (100 mg/kg i.p.). After tracheotomy, catheterize the arteria femoralis for blood pressure measurement. Administer the test substances as solution, orally by oesophageal tube or intravenously via the femoral vein in a suitable vehicle.

### B-5. PAH Model in the Anaesthetized Dog

In this animal model of pulmonary arterial hypertension (PAH), mongrel dogs having a body weight of about 25 kg are used. Narcosis is induced by slow i.v. administration of 25 mg/kg of sodium thiopental (Trapanal®) and 0.15 mg/kg of alcuronium chloride (Alloferin®) and maintained during the experiment by continuous infusion of 0.04 mg/kg/h of Fentanyl®, 0.25 mg/kg/h of droperidol (Dehydro-benzperidol®) and 15 μg/kg/h of alcuronium chloride (Alloferin®). Reflectory effects on the pulse by lowering of the blood pressure are kept to a minimum by autonomous blockage [continuous infusion of atropin (about 10 μg/kg/h) and propranolol (about 20 μg/kg/h)]. After intubation, the animals are ventilated using a ventilator with constant tidal volume such that an end-tidal CO₂ concentration of about 5% is reached. Ventilation takes place with ambient air enriched with about 30% oxygen (normoxa). For measuring the hemodynamic parameters, a liquid-filled catheter is implanted into the femoralis artery for measuring the blood pressure. A double-lumiger Swan-Ganz® catheter is introduced via the jugulara vein into the pulmonary artery (distal lumen for measuring the pulmonary arterial pressure, proximal lumen for measuring the central venus pressure). The left-ventricular pressure is measured following introduction of a micro-tip catheter (Millar® Instruments) via the carotis artery into the left ventricle, and from this, the dP/dt value is derived as a measure for the contractility. Substances are administered i.v. via the femoralis vein. The hemodynamic signals are recorded and evaluated using pressure sensors/amplifiers and PONEMAH® as data acquisition software.

To induce acute pulmonary hypertension, the stimulus used is either hypoxia or continuous infusion of thromboxan A₂ or a thromboxan A₂ analog. Acute hypoxia is induced by gradually reducing the oxygen in the ventilation air to about 14%, such that the mPAP increases to values of >25 mm Hg. If the stimulus used is a thromboxan A₂ analog, 0.21-0.32 μg/kg/min of U-46619 [9,11-dideoxy-9α,11α-epoxymethanoprostaglandin F_{2α} (from Sigma)] are infused to increase the mPAP to >25 mm Hg.

B-6. PAH Model in Anaesthetized Göttingen Minipiq

In this animal model of pulmonary arterial hypertension (PAH), Göttingen Minipigs having a body weight of about 25 kg are used. Narcosis is induced by 30 mg/kg of ketamine (Ketavet®) i.m., followed by i.v. administration of 10 mg/kg of sodium thiopental (Trapanal®); during the experiment, it is maintained by inhalation narcosis using enfluran (2-2.5%) in a mixture of ambient air enriched with about 30-35% oxygen/N₂O (1:1.5). For measuring the hemodynamic parameters, a liquid-filled catheter is implanted into the coratid artery for measuring the blood pressure. A double-lumiger Swan-Ganz® catheter is introduced via the jugulara vein into the pulmonary artery (distal lumen for measuring the pulmonary arterial pressure, proximal lumen for measuring the central venus pressure). The left-ventricular pressure is measured following introduction of a micro-tip catheter (Millar® Instruments) via the carotis artery into the left ventricle, and from this, the dP/dt value is derived as a measure for the contractility. Substances are administered i.v. via the femoralis vein. The hemodynamic signals are recorded and evaluated using pressure sensors/amplifiers and PONEMAH® as data acquisition software.

To induce acute pulmonary hypertension, the stimulus used is continuous infusion of a thromboxan A₂ analog. Here, 0.12-0.14 μg/kg/min of U-46619 [9,11-dideoxy-9α,11α-epoxymethanoprostaglandin F_{2α} (from Sigma)] are infused to increase the mPAP to >25 mm Hg.

### C. EXEMPLARY EMBODIMENTS OF PHARMACEUTICAL COMPOSITIONS

The compounds of the invention can be converted into pharmaceutical preparations in the following ways:

### Tablet:

### Composition:

100 mg of the compound of the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

### Production:

The mixture of compound of the invention, lactose and starch is granulated with a 5% strength solution (m/m) of the PVP in water. The granules are mixed with the magnesium stearate for 5 minutes after drying. This mixture is compressed with a conventional tablet press (see above for format of the tablet). A guideline compressive force for the compression is 15 kN.

Suspension which can be Administered Orally:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound of the invention.

### Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

### Composition:

500 mg of the compound of the invention, 2.5 g of polysorbate and 97 g of polyethylene glycol 400.20 g of oral solution correspond to a single dose of 100 mg of the compound according to the invention.

### Production:

The compound of the invention is suspended in the mixture of polyethylene glycol and polysorbate with stirring. The stirring process is continued until the compound according to the invention has completely dissolved.

i.v. Solution:

The compound of the invention is dissolved in a concentration below the saturation solubility in a physiologically tolerated solvent (e.g. isotonic saline solution, 5% glucose solution and/or 30% PEG 400 solution). The solution is sterilized by filtration and used to fill sterile and pyrogen-free injection containers.

1. A compound of the formula (I) 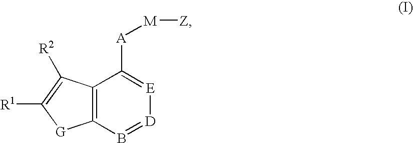
in which
B, D and E each represent CH or N,
G represents NH, O or S, with the proviso that
G does not represent O if at the same time B and E represent N and D represents CH,
and
G does not represent NH or S if at the same time B, D and E represent CH,
A represents O or N—R³ in which
R³ represents hydrogen, (C₁-C₆)-alkyl, (C₃-C₇)-cycloalkyl or (C₄-C₇)-cycloalkenyl,
M represents a group of the formula 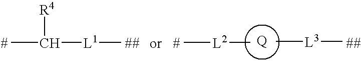
in which
# represents the point of attachment to the group A
and
## represents the point of attachment to the group Z,
R⁴ represents hydrogen or (C₁-C₄)-alkyl which may be substituted by hydroxyl or amino,
L¹ represents (C₁-C₇)-alkanediyl or (C₂-C₇)-alkenediyl which may be mono- or disubstituted by fluorine, or represents a group of the formula ♦-L^{1A}-V-L^{1B}-♦♦ in which
represents the point of attachment to the group —CHR⁴,
♦♦ represents the point of attachment to the group Z,
L^{1A} represents (C₁-C₅)-alkanediyl which may be mono- or disubstituted by identical or different substituents from the group consisting of (C₁-C₄)-alkyl and (C₁-C₄)-alkoxy,
L^{1B} represents a bond or (C₁-C₃)-alkanediyl which may be mono- or disubstituted by fluorine,
and
V represents O or N—R⁵ in which
R⁵ represents hydrogen, (C₁-C₆)-alkyl or (C₃-C₇)-cycloalkyl,
L² represents a bond or (C₁-C₄)-alkanediyl,
L³ represents (C₁-C₄)-alkanediyl which may be mono- or disubstituted by fluorine and in which a methylene group may be replaced by O or N—R⁶ in which
R⁶ represents hydrogen, (C₁-C₆)-alkyl or (C₃-C₇)-cycloalkyl,
or represents (C₂-C₄)-alkenediyl,
and
Q represents (C₃-C₇)-cycloalkyl, (C₄-C₇)-cycloalkenyl, phenyl, 5- to 7-membered heterocyclyl or 5- or 6-membered heteroaryl, each of which may be substituted by up to two identical or different radicals selected from the group consisting of fluorine, chlorine, (C₁-C₄)-alkyl, trifluoromethyl, hydroxyl, (C₁-C₄)-alkoxy, trifluoromethoxy, amino, mono-(C₁-C₄)-alkylamino and di-(C₁-C₄)-alkylamino,
where (C₁-C₄)-alkyl for its part may be substituted by hydroxyl, (C₁-C₄)-alkoxy, amino, mono- or di-(C₁-C₄)-alkylamino,
Z represents a group of the formula 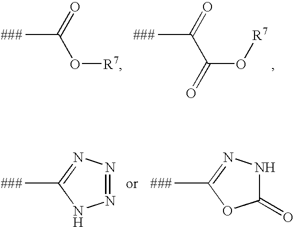
in which
### represents the point of attachment to the group L¹ or L³
and
R⁷ represents hydrogen or (C₁-C₄)-alkyl,
and
R¹ and R² are identical or different and independently of one another represent (C₃-C₇)-cyclo-alkyl, (C₄-C₇)-cycloalkenyl, phenyl, 5- to 7-membered heterocyclyl or 5- or 6-membered hetero-aryl each of which may be mono- to trisubstituted by identical or different radicals selected from the group consisting of halogen, cyano, nitro, (C₁-C₆)-alkyl, (C₂-C₆)-alkenyl, (C₂-C₄)-alkynyl, (C₃-C₇)-cycloalkyl, (C₄-C₇)-cycloalkenyl, (C₁-C₆)-alkoxy, trifluoromethyl, trifluoromethoxy, (C₁-C₆)-alkylthio, (C₁-C₆)-acyl, amino, mono-(C₁-C₆)-alkylamino, di-(C₁-C₆)-alkylamino and (C₁-C₆)-acylamino,
where (C₁-C₆)-alkyl and (C₁-C₆)-alkoxy for their part may each be substituted by cyano, hydroxyl, (C₁-C₄)-alkoxy, (C₁-C₄)-alkylthio, amino, mono- or di-(C₁-C₄)-alkylamino,
or
R¹ and/or R² represent phenyl in which two radicals attached to adjacent ring carbon atoms together form a group of the formula —O—CH₂—O—, —O—CHF—O—, —O—CF₂—O—, —O—CH₂—CH₂—O— or —O—CF₂—CF₂—O—,
or a salt thereof.

2. The compound of the formula (I) as claimed in claim 1 in which the bicyclic ring system 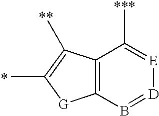
represents a heteroaryl group of the formula 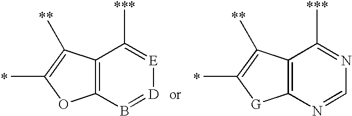
in which
represents the point of attachment to the radical R¹,
** represents the point of attachment to the radical R²
and
*** represents the point of attachment to the group -A-M-Z,
B, D and E each represent CH or N, with the proviso that B does not represent N if at the same time D represents CH and E represents N,
and
G represents NH or S,
A represents O or N—R³ in which
R³ represents hydrogen, (C₁-C₄)-alkyl or cyclopropyl,
M represents a group of the formula 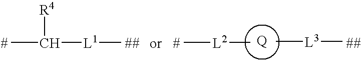
in which
# represents the point of attachment to the group A
and
## represents the point of attachment to the group Z,
R⁴ represents hydrogen or (C₁-C₃)-alkyl which may be substituted by hydroxyl or amino,
L¹ represents (C₃-C₇)-alkanediyl or (C₃-C₇)-alkenediyl which may be mono- or disubstituted by fluorine, or a group of the formula ♦-L^{1A}-V-L^{1B}-♦♦ in which
represents the point of attachment to the group —CHR⁴,
♦♦ represents the point of attachment to the group Z,
L^{1A} represents (C₁-C₃)-alkanediyl which may be mono- or disubstituted by identical or different substituents from the group consisting of methyl and ethyl,
L^{1B} represents (C₁-C₃)-alkanediyl which may be mono- or disubstituted by fluorine,
and
V represents O or N—R⁵ in which
R⁵ represents hydrogen, (C₁-C₃)-alkyl or cyclopropyl,
L² represents a bond or (C₁-C₃)-alkanediyl,
L³ represents (C₁-C₃)-alkanediyl which may be mono- or disubstituted by fluorine, (C₂-C₃)-alkenediyl or a group of the formula —W—CR⁸R⁹—, —W—CH₂—CR⁸R⁹— or —CH₂—W—CR⁸R⁹— in which
 represents the point of attachment to the ring Q,
 represents the point of attachment to the group Z,
W represents O or N—R⁶ in which
R⁶ represents hydrogen, (C₁-C₃)-alkyl or cyclopropyl,
and
R⁸ and R⁹ independently of one another represent hydrogen or fluorine,
and
Q represents (C₄-C₆)-cycloalkyl, (C₄-C₆)-cycloalkenyl, phenyl or 5- or 6-membered heterocyclyl, each of which may be up substituted by up to two identical or different radicals selected from the group consisting of fluorine, chlorine, (C₁-C₃)-alkyl, trifluoromethyl, hydroxyl, methoxy, ethoxy, trifluoromethoxy, amino, methylamino, ethylamino, dimethylamino and diethylamino,
Z represents a group of the formula 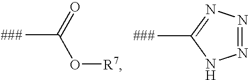
in which
### represents the point of attachment to the group L¹ or L³
and
R⁷ represents hydrogen, methyl or ethyl,
and
R¹ and R² are identical or different and independently of one another represent (C₄-C₆)-cyclo-alkenyl, phenyl or 5- or 6-membered heteroaryl, each of which may be mono- or disubstituted by identical or different radicals selected from the group consisting of fluorine, chlorine, cyano, (C₁-C₅)-alkyl, (C₂-C₅)-alkenyl, (C₃-C₆)-cycloalkyl, (C₄-C₆)-cycloalkenyl, (C₁-C₄)-alkoxy, trifluoromethyl, trifluoromethoxy, (C₁-C₄)-alkylthio, (C₁-C₅)-acyl, amino, mono-(C₁-C₄)-alkyl-amino, di-(C₁-C₄)-alkylamino and (C₁-C₄)-acylamino,
or
R¹ and/or R² represent phenyl in which two radicals attached to adjacent ring carbon atoms together form a group of the formula —O—CH₂—O—, —O—CHF—O— or —O—CF₂—O—.

3. The compound of the formula (I) as claimed in claim 1 in which the bicyclic ring system 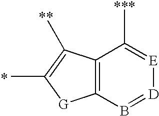
represents a heteroaryl group of the formula 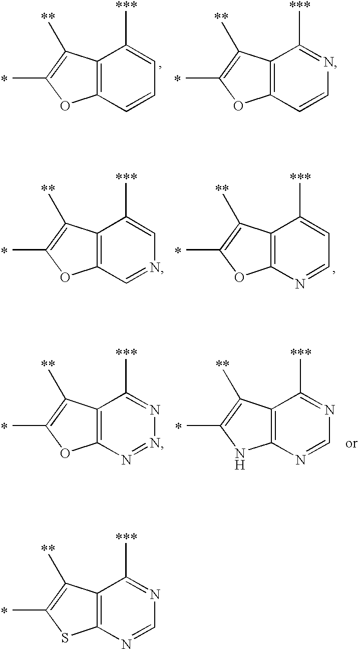
in which
represents the point of attachment to the radical R¹,
** represents the point of attachment to the radical R²
and
*** represents the point of attachment to the group -A-M-Z,
A represents O or NH,
M represents a group of the formula 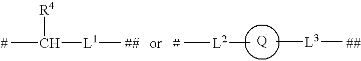
in which
# represents the point of attachment to the group A
and
## represents the point of attachment to the group Z,
R⁴ represents hydrogen, methyl or ethyl,
L¹ represents (C₃-C₇)-alkanediyl, (C₃-C₇)-alkenediyl or a group of the formula ♦-L^{1A}-V-L^{1B}-♦♦ in which
represents the point of attachment to the group —CHR⁴,
♦♦ represents the point of attachment to the group Z,
L^{1A} represents (C₁-C₃)-alkanediyl which may be mono- or disubstituted by methyl,
L^{1B} represents (C₁-C₃)-alkanediyl
and
V represents O or N—CH₃,
L² represents a bond, methylene, ethane-1,1-diyl or ethane-1,2-diyl,
L³ represents (C₁-C₃)-alkanediyl or a group of the formula —W—CH₂— or —W—CH₂—CH₂— in which
 represents the point of attachment to the ring Q,
 represents the point of attachment to the group Z
and
W represents O or N—R⁶ in which
R⁶ represents hydrogen or (C₁-C₃)-alkyl,
and
Q represents cyclobutyl, cyclopentyl, cyclopentenyl, cyclohexyl, pyrrolidinyl, piperidinyl, tetrahydrofuranyl, tetrahydropyranyl, morpholinyl or phenyl, each of which may be substituted by up to two identical or different radicals selected from the group consisting of fluorine, methyl, ethyl, trifluoromethyl, hydroxyl, methoxy and ethoxy,
Z represents a group of the formula 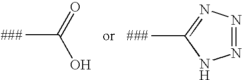
in which
### represents the point of attachment to the group L¹ or L³,
and
R¹ and R² are identical or different and independently of one another represent cyclopenten-1-yl, cyclohexen-1-yl, phenyl, thienyl or pyridyl, each of which may be mono- or disubstituted by identical or different radicals selected from the group consisting of fluorine, chlorine, cyano, (C₁-C₄)-alkyl, (C₂-C₄)-alkenyl, (C₁-C₄)-alkoxy, trifluoromethyl and trifluoromethoxy.

4. The compound as claimed in claim 1,
in which
B, D, and E each represent CH,
G represents O,
A represents O or NH,
M represents a group of the formula 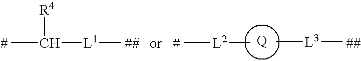
in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
represents the point of attachment to the group —CHR⁴,
♦♦ represents the point of attachment to the carboxylic acid grouping,
L^{1A} represents methylene or ethane-1,2-diyl which may be mono- or disubstituted by methyl,
and
L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
 represents the point of attachment to the ring Q
and
 represents the point of attachment to the carboxylic acid grouping,
and
Q represents cyclopentyl, cyclohexyl or phenyl,
Z represents a group of the formula 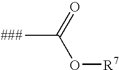
in which
R⁷ represented hydrogen,
R¹ represents phenyl which may be substituted by fluorine or chlorine,
and
R² represents phenyl which may be substituted by methyl, ethyl, methoxy or ethoxy.

5. The compound as claimed in claim 1,
in which
B and D each represent CH,
E represents N,
G represents O,
A represents O or NH,
M represents a group of the formula 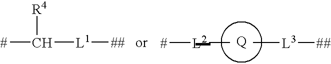
in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
represents the point of attachment to the group —CHR⁴,
♦♦ represents the point of attachment to the carboxylic acid grouping,
L^{1A} represents methylene or ethane-1,2-diyl which may be mono- or disubstituted by methyl,
and
L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
 represents the point of attachment to the ring Q
and
 represents the point of attachment to the carboxylic acid grouping,
and
Q represents cyclopentyl, cyclohexyl or phenyl,
Z represents a group of the formula 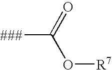
in which
R⁷ represented hydrogen,
R¹ represents phenyl which may be substituted by fluorine or chlorine,
and
R² represents phenyl which may be substituted by methyl, ethyl, methoxy or ethoxy.

6. The compound as claimed in claim 1,
in which
B represents N
D and E each represent CH,
G represents O,
A represents O or NH,
M represents a group of the formula 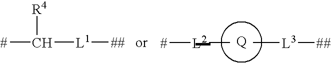
in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦ in which
represents the point of attachment to the group —CHR⁴,
♦♦ represents the point of attachment to the carboxylic acid grouping,
L^{1A} represents methylene or ethane-1,2-diyl which may be mono- or disubstituted by methyl,
and
L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
 represents the point of attachment to the ring Q
and
 represents the point of attachment to the carboxylic acid grouping,
and
Q represents cyclopentyl, cyclohexyl or phenyl,
Z represents a group of the formula 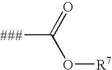
in which
R⁷ represented hydrogen,
R¹ represents phenyl which may be substituted by fluorine or chlorine,
and
R² represents phenyl which may be substituted by methyl, ethyl, methoxy or ethoxy.

7. The compound as claimed in claim 1,
in which
B and E each represent N,
D represents CH,
G represents NH,
A represents O or NH,
M represents a group of the formula 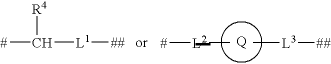
in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦
in which
represents the point of attachment to the group —CHR⁴,
♦♦ represents the point of attachment to the carboxylic acid grouping,
L^{1A} represents methylene or ethane-1,2-diyl which may be mono- or disubstituted by methyl,
and
L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
 represents the point of attachment to the ring Q
and
 represents the point of attachment to the carboxylic acid grouping,
and
Q represents cyclopentyl, cyclohexyl or phenyl,
Z represents a group of the formula 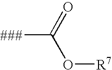
in which
R⁷ represented hydrogen,
R¹ represents phenyl which may be substituted by fluorine or chlorine,
and
R² represents phenyl which may be substituted by methyl, ethyl, methoxy or ethoxy.

8. The compound as claimed in claim 1,
in which
B and E each represent N,
D represents CH,
G represents S,
A represents O or NH,
M represents a group of the formula 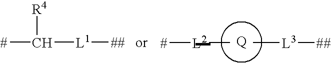
in which
# represents the point of attachment to the group A
and
## represents the point of attachment to the carboxylic acid grouping,
R⁴ represents hydrogen or methyl,
L¹ represents butane-1,4-diyl, pentane-1,5-diyl or a group of the formula ♦-L^{1A}-O-L^{1B}-♦♦
in which
represents the point of attachment to the group —CHR⁴,
♦♦ represents the point of attachment to the carboxylic acid grouping,
L^{1A} represents methylene or ethane-1,2-diyl which may be mono- or disubstituted by methyl,
and
L^{1B} represents methylene or ethane-1,2-diyl,
L² represents a bond or methylene,
L³ represents methylene, ethane-1,2-diyl, propane-1,3-diyl or a group of the formula —O—CH₂— or —O—CH₂—CH₂— in which
 represents the point of attachment to the ring Q
and
 represents the point of attachment to the carboxylic acid grouping,
and
Q represents cyclopentyl, cyclohexyl or phenyl,
Z represents a group of the formula 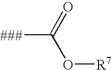
in which
R⁷ represented hydrogen,
R¹ represents phenyl which may be substituted by fluorine or chlorine,
and
R² represents phenyl which may be substituted by methyl, ethyl, methoxy or ethoxy.

9. A process for preparing compounds as defined in claim 1 in which Z represents —COOH or —C(═O)—COOH, characterized in that either
[A] compounds of the formula (II) 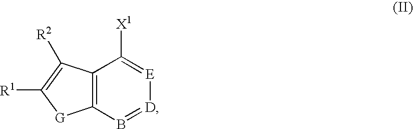
in which B, D, E, G, R¹ and R² each have the meanings given in claim 1 and
X¹ represents a leaving group such as, for example, halogen, in particular chlorine, are reacted in an inert solvent in the presence of a base with a compound of the formula (III) 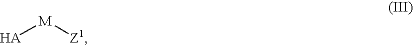
in which A and M have the meanings given in claim 1 and
Z¹ represents cyano or a group of the formula —[C(O)]_{y}—COOR^{7A} in which
y represents the number 0 or 1
and
R^{7A} represents (C₁-C₄)-alkyl,
to give compounds of the formula (IV) 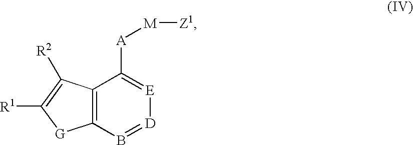
in which A, B, D, E, G, M, Z¹, R¹ and R² each have the meanings given above,
or
[B] compounds of the formula (V) 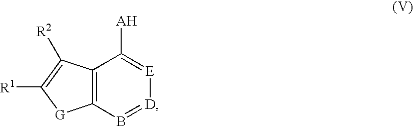
in which A, B, D, E, G, R¹ and R² each have the meanings given in claim 1, are reacted in an inert solvent in the presence of a base with a compound of the formula (VI) 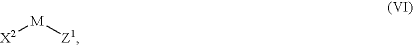
in which M has the meanings given in claim 1 and Z¹ has the meaning given above and
X² represents a leaving group such as, for example, halogen, mesylate, tosylate or triflate, to give compounds of the formula (IV) 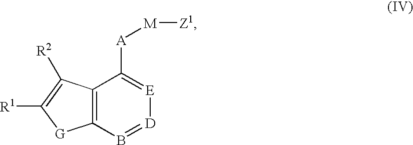
in which A, B, D, E, G, M, Z¹, R¹ and R² each have the meanings given above, and the compounds of the formula (IV) are then converted by hydrolysis of the ester or cyano group Z¹ into the carboxylic acids of the formula (Ia) 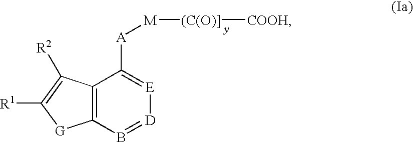
in which A, B, D, E, G, M, R¹, R² and y each have the meanings given above, and these are, if appropriate, converted into their salts using the appropriate (i) solvents and/or (ii) bases or acids.

10-11. (canceled)

12. A medicament comprising a compound as defined in claim 1 in combination with an inert non-toxic pharmaceutically acceptable auxiliary.

13-14. (canceled)

15. A method for the treatment and/or prophylaxis of angina pectoris, pulmonary hypertension, thromboembolic disorders and peripheral occlusive diseases in humans and animals by administering an effective amount of at least one compound as defined in claim 1.
